(19) United States
(12) Patent Application Publication
(10) Pub. No.: US 2003/0219787 A1
(43) Pub. Date: Nov. 27, 2003

(54) Novel human gene functionally related to dyslexia

(76) Inventors: Juha Kere, Espoo (FI); Mikko Taipale, Heidelberg (DE); Jaana Nopola-Hemmi, Helsinki (FI); Nina Kaminen, Helsinki (FI)

Correspondence Address: BIRCH STEWART KOLASCH & BIRCH, PO BOX 747, FALLS CHURCH, VA, 22040-0747, US

(21) Appl. No.: 10/364,505

(22) Filed: Feb. 12, 2003

Related U.S. Application Data
(60) Provisional application No. 60/355,782, filed on Feb. 12, 2002.

(51) Int. Cl. C12Q 1/68; C07H 21/04; C12N 9/00; C12P 21/02; C12N 5/06

(52) U.S. Cl. 435/6; 435/69.1; 435/320.1; 435/325; 435/183; 536/23.2

(57) ABSTRACT

The present invention describes a novel human gene, DYXC1, which is functionally related to dyslexia. DYXC1 gene encodes a 420-amino acid residue protein. DYXC1 is expressed in several tissues, including the brain, and is localized in the nucleus. In addition, four single nucleotide polymorphisms (SNPs) in DYXC1 mRNA have been characterized in this invention. The invention provides diagnostic methods and materials for analysing allelic variation in DYXC1 gene. This invention also provides polypeptides encoded by DYXC1 gene and antibodies binding to said polypeptides.

The present invention relates to a novel human gene functionally related to dyslexia, especially variant forms (e.g. alleles) thereof that predispose an individual to develop dyslexia. Thus, this invention also relates to the polymorphism of said gene as well as diagnostic methods and materials for analysing allelic variation in said gene. This invention also provides polypeptides encoded by said gene and antibodies binding to said polypeptides. The materials of the invention can be used to study the brain processes such as reading, phonological processing, rapid naming and verbal short term memory.

### BACKGROUND OF THE INVENTION

Dyslexia, or specific reading disability, is the most common childhood learning disorder. It is estimated that about 3-10% of people have specific difficulties in reading, despite adequate intelligence, education and social environment. Several different theories have been put forth to account for the diverse symptoms seen in dyslexic subjects. At present, it is thought that dyslexia is primarily a phonological deficit, emphasizing the linguistic basis of this condition (1-3). However, it is possible that dyslexia is not specific to language. Rather, it may result from a deficit in processing fast temporal data, be it visual or auditory. This temporal processing deficit would, consequently, manifest itself primarily as dyslexia (4).

Available evidence suggests that dyslexia is a neurological disorder with a genetic basis. Functional brain imaging studies have illustrated that dyslexia has universal neurobiological correlates (5). There is extensive evidence of genetic factors which contribute to dyslexia. There are significant differences, however, in the heritability of different components of dyslexia (6). Linkage and association studies have pinpointed several loci for dyslexia. In particular, two loci have been promising. DYX1 in chromosome 15q21 was the first locus to be associated with dyslexia (7), and the results have been replicated in three independent studies thereafter (8-10). The presence of a second dyslexia locus, DYX2, in chromosome 6p21 has also been established (11).

We have previously reported a translocation t(2;15)(q11;q21) which segregates with dyslexia (12). In present invention, we have cloned the breakpoint and narrowed down the breakpoint interval within a 3229 bp region with Southern hybridization. This region contains a 301 bp AT rich sequence. Considering that AT rich repeats are known to occur at many chromosomal rearrangement sites (13), the 301 bp AT rich sequence is likely to be the exact breakpoint site. Furthermore, we have unexpectedly discovered and characterized a novel gene residing in the breakpoint region, which gene we named DYXC1 and which is causally correlated with dyslexia.

### SUMMARY OF THE INVENTION

The present invention describes a novel human gene, DYXC1, which is causally correlated with dyslexia. The coding sequence of DYXC1 is 1260 bp in length (SEQ ID NO: 1), and it encodes a predicted protein of 420 amino acids (SEQ ID NO: 3). DYXC1 is expressed in several tissues, most abundantly in brain, lung, kidney and testis. The predicted 420 amino-acid protein contains three C-terminal tetratricopeptide repeat (TPR) domains, thought to mediate protein-protein interactions. Besides these domains, it bears no similarity to known proteins. Transfection and immunofluorescence studies indicate that DYXC1 is a nuclear protein.

The coding sequence of DYXC1 was predicted from the genomic sequence of BAC clones RP11-178D12 and CTD-2137J4. The length of DYXC1 mRNA is 1993 bp (SEQ ID NO: 2), and it encodes a predicted protein of 420 amino acids. DYXC1 consists of 10 exons spanning approximately 78 kb of genomic DNA (FIG. 1D). The start codon (AUG) of DYXC1 is located 369 bp from the predicted transcription initiation site in exon 2. Putative promoter of DYXC1 has a TATA box (TATAAAT) at position −31.

In one aspect, the invention features isolated DYXC1 nucleic acid molecules having the sequence of SEQ ID NO: 1 or a complement thereof; homologs and variants thereof as well as fragments thereof. In a preferred embodiment the isolated DYXC1 nucleic acid is mammalian. In an even more preferred embodiment the isolated DYXC1 nucleic acid is human. The invention features also vectors comprising the disclosed nucleic acid as well as host cells for the expression or amplification of such vectors.

In addition, we have characterized in this invention five single nucleotide polymorphisms (SNPs) in DYXC1 mRNA. One sequence variant (1249G→T) introduces a premature stop codon and is inherited with dyslexia in a three-generation family. The frequency of the polymorphism is significantly (p=0.0278) elevated in dyslexic subjects, compared to control samples. The polymorphism truncates the predicted DYXC1 protein by four amino acids, suggesting that it is a functional SNP. Thus, in another aspect, the invention features nucleic acids comprising at least one single nucleotide polymorphism in any one of the following positions as defined by SEQ ID NO: 1: position 4 (C preferably to T), 572 (G preferably to A), 1249 (G preferably to T), 1259 (C preferably to G); and SEQ ID NO: 2: position 205 (C preferably to T).

The invention further provides polypeptides encoded by DYXC1 gene or allelic variants thereof and antibodies binding to said polypeptides. The invention also relates to diagnostic methods, kits and materials for analysing allelic variation in DYXC1 gene and its cellular function.

### DESCRIPTION OF THE DRAWINGS

FIGS. 1A, 1B, 1C and 1D. 1A, Pedigree of the studied family. Black fill denotes translocation, grey fill dyslexia. 1B, Fluorescent in situ hybridization with BAC clone 178D12 as a probe, showing hybridisation signals in chromosomes 15, der(15), and der(2). 1C, Southern hybridization with a probe derived from 178D12 shows genomic rearrangement with six restriction enzymes in the studied sample (T) compared to the control sample (C). 1D, Physical map of the breakpoint region, including DYXC1 (black) and an intronic pseudogene (white), drawn to scale. Black triangle-illustrates the Southern hybridisation probe position, grey bar denotes the breakpoint interval.

FIGS. 2A, 2B, 2C, 2D and 2E. 2A, Comparison of the protein sequences of human DYXC1 and mouse mdyxc1. The SNPs found in this study are marked with a circumflex accent, and the three TPR domains are marked with asterisks. 2B, RT-PCR from human multiple tissue cDNA panels I and II (Clontech). Lanes: 1)λ/φX174 size marker, 2) heart, 3) brain, 4) placenta, 5) lung, 6) liver, 7) skeletal muscle, 8) kidney, 9) pancreas, 10) spleen, 11) thymus, 12) prostate, 13) testis, 14) ovary, 15) small intestine, 16) colon, and 17) leukocyte. 2C and 2D, DYXC1 Northern blot from Multiple Tissue Northern (MTN) Blot panels I and II (Clontech). Lanes in FIG. 2C: 1) heart, 2) brain, 3) placenta, 4) lung, 5) liver, 6) skeletal muscle, 7) kidney, 8) pancreas; Lanes in FIG. 2D: 9) spleen, 10) thymus, 11) prostate, 12) testis, 13) ovary, 14) small intestine, 15) colon, and 16) leukocyte. 2E, Cellular localization of DYXC1 protein. Cos-1 cells transfected with DYXC1-V5 fusion construct were stained with monoclonal mouse α-V5 antibody and FITC-conjugated α-mouse-IgG (grey). DAPI stained nuclei are shown in light grey.

FIG. 3. Pedigree of the family in which two DYXC1 polymorphisms are transmitted with dyslexia. Alleles for four loci are shown below individuals. Black rectangle indicates the halotype transmitted with dyslexia in this family.

### DETAILED DESCRIPTION OF THE INVENTION

This invention is based on the discovery and characterization of a novel human gene termed DYXC1. The human DYXC1 gene is 1260 bp in length (SEQ ID NO: 1) and it encodes a 420-amino acid residue protein (SEQ ID NO: 3). The cDNA of total DYXC1 mRNA (SEQ ID NO: 2) has been deposited in GenBank with accession number AF337549. DYXC1 maps to human chromosome 15q21. The present invention shows that previously reported balanced translocation breakpoint t(2;15)(q11;q21) segregating coincidentally with developmental dyslexia is located in DYXC1 thus indicating that DYXC1 is linked to dyslexia. In addition, it was unexpectedly discovered in the present invention that point mutations, i.e. SNPs, in DYXC1 segregate with the susceptibility to develop dyslexia.

The present invention provides DYXC1 nucleic acids, homologs thereof and fragments thereof. The human DYXC1 cDNA sequence is disclosed in SEQ ID NO: 1. Preferred homologs, such as mouse dyxc1 (SEQ ID NO: 4), have a sequence at least about 79% homologous with a nucleotide sequence of SEQ ID NO: 1. In a preferred embodiment, the DYXC1 nucleic acid is from a mammal, e.g. a mouse or human. In another preferred embodiment the nucleic acid has the sequence of SEQ ID NO: 1 a complement thereof or a fragment thereof. In one embodiment of the invention the fragment disclosed can be a primer or probe, which is capable to hybridise specifically to the DYXC1 nucleic acids described herein. The preparation and modification of primers and probes capable of binding to a known nucleic acid are well-established techniques in the art (see, for example, Current Protocols in Molecular Biology, eds. Ausubel et al, John Wiley & Sons: 1992). Generally, a primer or a probe is a substantially purified oligonucleotide being 12 to 60 nucleotides long, preferably 16 to 40 nucleotides. A primer or probe need not reflect the exact sequence of a template, i.e. a target nucleic acid, but must be sufficiently complementary to hybridise with the template under stringent conditions.

The invention also involves nucleotide sequence variants capable of encoding DYXC1 polypeptides. Such variants include sequences that differ from the disclosed DYXC1 nucleic acids by one or more nucleotide substitutions, additions or deletions, such as allelic variants. Said nucleotide substitutions may also arise due to the degeneracy of the genetic code. The nucleic acids of the invention can also be described as capable of hybridising under stringent conditions to the nucleic acid sequence of SEQ ID NO: 1 or 2 or a complement thereof. Such stringent DNA hybridisation conditions are well-known in the art, e.g. 6×NaCl/sodium citrate (SSC) at about 45° C. is applied for a hybridisation step, followed by a wash of 2×SSC at 50° C. or, e.g., alternatively hybridization at 42° C. in 5×SSC, 20 mM NaPO4, pH 6.8, 50% formamide; and washing at 42° C. in 0.2×SSC. Those skilled in the art understand that it is desirable to vary these conditions empirically based on the length and the GC nucleotide base content of the sequences to be hybridised, and that formulas for determining such variation exist (See, for example, Sambrook et al, “Molecular Cloning: A Laboratory Manual”, Second Edition, pages 9.47-9.51, Cold Spring Harbor, N.Y.: Cold Spring Harbor Laboratory Press (1989)). Nucleic acids of the invention, fragments thereof and variants thereof with sufficient similarity to the non-coding strand of said nucleic acids to hybridise thereto under stringent conditions are useful for identifying, purifying, and isolating nucleic acids encoding other, non-human, mammalian forms of DYXC1. Thus, such polynucleotide fragments and variants are intended as aspects of the invention.

The present invention also provides plasmids and vectors encoding an DYXC1 polypeptide, which constructs can be used in the expression of said DYXC1 polypeptide in or from a host cell. The selecting of a suitable plasmid or vector for a certain use is within the abilities of a skilled artisan. As the host cell may be any prokaryotic or eukaryotic cell, a plasmid or vector encoding an DYXC1 polypeptide can be used to the production of said DYXC1 polypeptide as a recombinant protein via microbial or eukaryotic cellular processes. Typically, said plasmids and vectors comprises a ligated nucleic acid encoding a recombinant protein, said nucleic acid operably linked to at least one transcriptional regulatory sequence (See, for example, Sambrook et al, “Molecular Cloning: A Laboratory Manual”, Second Edition, Cold Spring Harbor, N.Y.: Cold Spring Harbor Laboratory Press (1989)).

The present invention further describes the characterization of single nucleotide polymorphisms (SNPs) in human DYXC1 gene. SNPs can be used in mapping the human genome and, when a SNP is linked with a disease or condition, to clarify genetic basis of the disease or condition, in this particular case, at least of dyslexia. In this invention we have characterized five single nucleotide polymorphisms (SNPs) in DYXC1 mRNA (SEQ ID NOS: 1 and 2). Accordingly, the present invention provides an DYXC1 nucleic acid comprising a SNP in any one of the following positions in the nucleic acid sequence of SEQ ID NO: 1: position 4; 572; 1249 or 1259. Allelic variation at position 4 consists of a single base substitution from C preferably to T. Allelic variation at position 572 consists of a single base substitution from G preferably to A. Allelic variation at position 1249 consists of a single base substitution from G preferably to T. Allelic variation at position 1259 consists of a single base substitution from C preferably to G. The present invention also describes a SNP in DYXC1 mRNA at position −164 outside the coding sequence of DYXC1. This position corresponds to position 205 set forth in SEQ ID NO: 2. Allelic variation at position −164 consists of a single base substitution from C preferably to T.

The SNP variant of DYXC1, wherein the single base substitution is at position 1249 (G→T), introduces a premature stop codon and is inherited with dyslexia in a three-generation family. The frequency of the polymorphism is significantly (p=0.0278) elevated in dyslexic subjects, compared to control samples as shown in Examples. The polymorphism truncates the predicted DYXC1 protein by four amino acids, suggesting that it is a functional SNP.

Further, new polymorphic gene regions in DYXC1 nucleic acids can be identified by determining the DYXC1 nucleic acid sequences in population of individuals. If new polymorphic region (e.g. SNP) is found, then the link with a specific disease can be determined by studying specific populations of individuals, such as dyslexics. A polymorphic site or region may be located in any part of a gene, e.g., exons, introns and promoter region.

The present invention makes available DYXC1 polypeptides. Such polypeptides can be recombinant proteins produced by, e.g., the host cells described hereinabove, said recombinant proteins being isolated from other cellular proteins. Preferably, said polypeptides have an amino acid which is at least about 78% identical or homologous to human DYXC1 protein of sequence set forth in SEQ ID NO: 3. In a preferred embodiment, an DYXC1 polypeptide of the present invention is mammalian, e.g. murine or human, DYXC1 protein. In addition, the present invention provides splice variants of DYXC1 protein.

An DYXC1 polypeptide of the invention can also be used as an antigen to produce antibodies. Techniques of preparing antisera, poly- or monoclonal antibodies are well-known protocols in the art (see, for example, Antibodies: A laboratory Manual, eds. Harlow and Lane, Cold Spring Harbor Laboratory Press: 1988). Thus, the present invention makes available DYXC1 specific antibodies. Especially, the antibodies, of the invention can be labeled with a detectable label and used in the determination of the presence of DYXC1 polypeptides in a sample, e.g. for diagnosis of dyslexia.

The present invention further provides means for prognostic or diagnostic assays for determining if a subject has or is likely to develop dyslexia, which is associated with the variation or dysfunction of DYXC1. Basically, such assays comprise a detection step, wherein the presence or absence of a genetic alteration or defect in DYXC1 is determined in a biological sample from the subject. Said detection step can be performed, e.g., by methods involving sequence analysis, nucleic acid hybridisation, primer extension, restriction enzyme site mapping or antibody binding. These methods are well-known in the art (see, for example, Current Protocols in Molecular Biology, eds. Ausubel et al, John Wiley & Sons:1992).

In particular, the present invention is directed to a method of determining the presence or absence of an DYXC1 SNP of the invention in a biological sample from a human for diagnostics of dyslexia or for assessing the predisposition of an individual to dyslexia. Said method comprises determining the sequence of the nucleic acid of a human at one or more positions 4, 572, 1249 and 1259 in the DYXC1 gene or mRNA as defined in SEQ ID NO: 1 and position 205 as defined by SEQ ID NO: 2 and determining the status of the human by reference to polymorphism in DYXC1 gene. In a preferred embodiment the sample is contacted with oligonucleotide primers so that the nucleic acid region containing the potential single nucleotide polymorphism is amplified by polymerase chain reaction prior to determining the sequence. The final results can be obtained by using a method selected from, e.g., allele specific nucleic acid amplification, allele specific nucleic acid hybridisation, oligonucleotide ligation assay or restriction fragment length polymorphism (RFLP). These methods are well-known for a skilled person of the art (see, for example, Current Protocols in Molecular Biology, eds. Ausubel et al, John Wiley & Sons: 1992, or Landegren et al, “Reading Bits of Genetic Information: Methods for Single-Nucleotide Polymorphism Analysis”, Genome Research 8:769-776).

The invention also features diagnostic or prognostic kits for use in detecting the presence of DYXC1 SNP in a biological sample. The kit provides means for the diagnostics of dyslexia or for assessing the predisposition of an individual to dyslexia mediated by variation or dysfunction of DYXC1. The kit can comprise a labeled compound capable of detecting DYXC1 polypeptide or nucleic acid (e.g. mRNA) in a biological sample. The kit can also comprise nucleic acid primers or probes capable of hybridising specifically to at least of portion of an DYXC1 gene or allelic variant thereof. The kit can be packaged in a suitable container and preferably it contains instructions for using the kit.

It is also realised in the present invention that transgenic non-human animals, such as transgenic mice, which include a heterologous form of an DYXC1 gene, can be designed and produced utilising the disclosure presented herein (see, for example, Manipulating the Mouse Embryo: A laboratory Manual, eds. Hogan et al, Cold Spring Harbor Laboratory Press, 1986). Such transgenic animals can be useful as animal models for studying, e.g., the function of DYXC1 gene and alleles thereof, or for expressing recombinant DYXC1 polypeptides.

A further embodiment of the present invention is a method for identifying a mutant DYXC1 nucleotide sequence in a suspected mutant DYXC1 allele which comprises comparing the nucleotide sequence of the suspected mutant DYXC1 allele with a wild-type DYXC1 nucleotide sequence or a part thereof, wherein a difference between the suspected mutant and the wild-type sequence identifies a mutant DYXC1 nucleotide sequence. In said method the sequence of said suspected mutant DYXC1 allele can be compared with the sequence of one or more wild-type DYXC1 gene sequences selected from the sequences set forth in SEQ ID NO: 1, SEQ ID NO: 2, SEQ ID NO: 6, SEQ ID NO: 7, SEQ ID NO: 8, SEQ ID NO: 9, SEQ ID NO: 10 and wild-type allelic variants thereof. For the screening of new point mutations, deletion mutations and insertion mutations in DYXC1 a plentiful of techniques well-known for a skilled artisan can be utilised, such as methods involving sequence analysis, nucleic acid hybridisation, primer extension, restriction enzyme site mapping and particularly methods described below in Examples and Materials and Methods.

### EXAMPLES

### Example 1

Fine Mapping of the Translocation Breakpoint

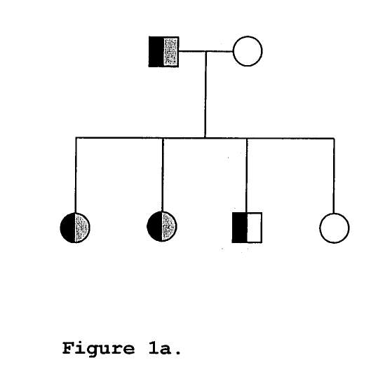

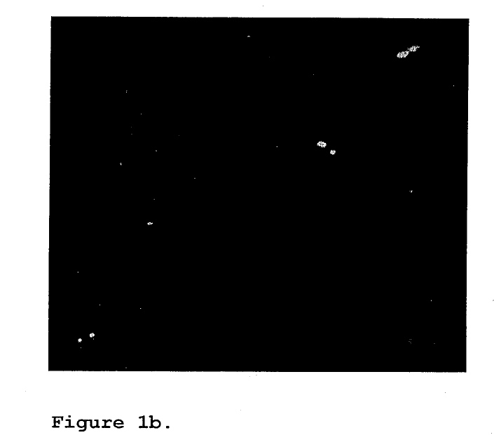

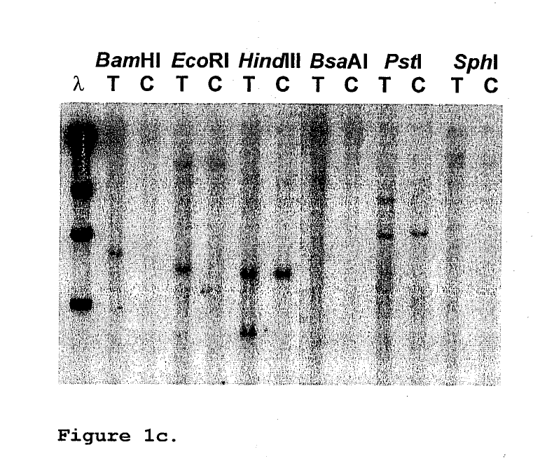

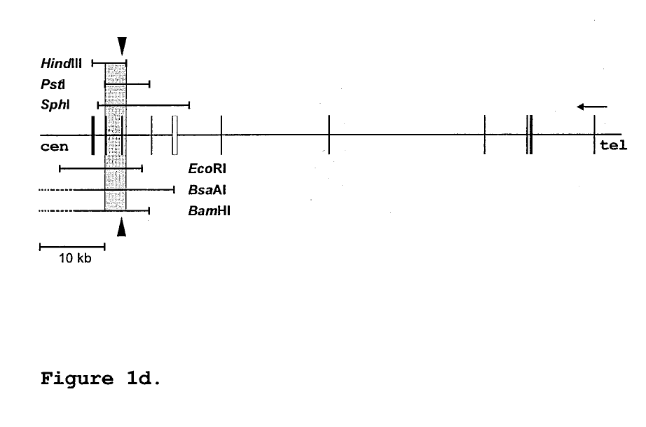

The translocation and the phenotypes of the members of the family studied here (FIG. 1A) have been described previously (12). Fluorescent in situ hybridization (FISH) restricted the location of the translocation breakpoint within the BAC clone RP-11-178D12 (AC013355; FIG. 1B). The clone contained two known genes, cell-cycle restoration protein 8 (CPR8) and complementation class B phosphoinositol glycan (PIG-B), in addition to the genes described herein. To further localize the breakpoint, we used amplified non-repetitive genomic DNA fragments from the BAC clone RPCI-11-178D12 as probes in Southern hybridization. A probe corresponding to nucleotides 102317-102837 of the complete sequence of 178D12 revealed a genomic rearrangement with 6 different restriction enzymes (FIG. 1C). Thus, we could pinpoint the breakpoint to a region of 3229 bp, limited by the restriction sites for PstI and HindIII (FIG. 1D). The breakpoint region includes exons 8 and 9 of a novel gene, DYXC1 (see below).

### Example 2

Characterization of DYXC1

The coding sequence of DYXC1 was predicted in silico from the genomic sequence of BAC clones RP11-178D12 and CTD-2137J4. Exon-intron boundaries were confirmed with RT-PCR. The length of DYXC1 mRNA, obtained by RT-PCR, is 1993 bp, and it encodes a predicted protein of 420 amino acids. DYXC1 consists of 10 exons spanning approximately 78 kb of genomic DNA (FIG. 1D). Three promoter prediction programs (see below) identified a promoter precisely before the 5′ end of the RT-PCR obtained mRNA, suggesting that the cloned mRNA is nearly complete. The start codon (AUG) of DYXC1 is located 369 bp from the predicted transcription initiation site in exon 2. Putative promoter of DYXC1 has a TATA box (TATAAAT) at position −31.

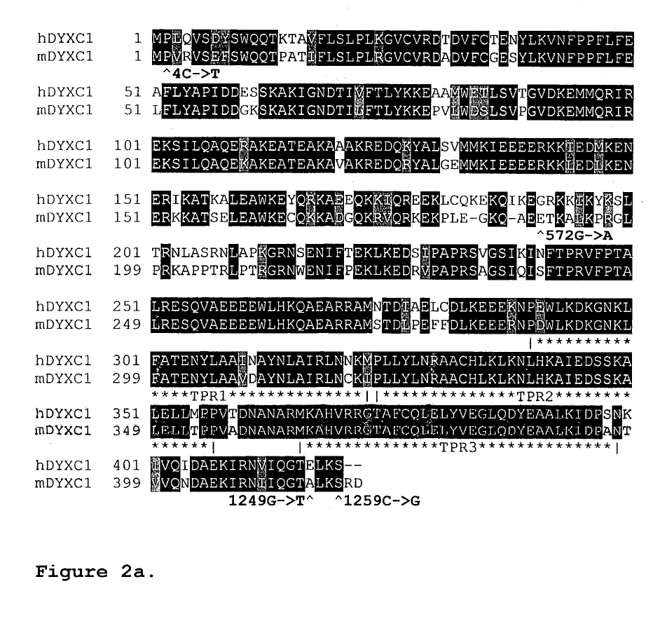

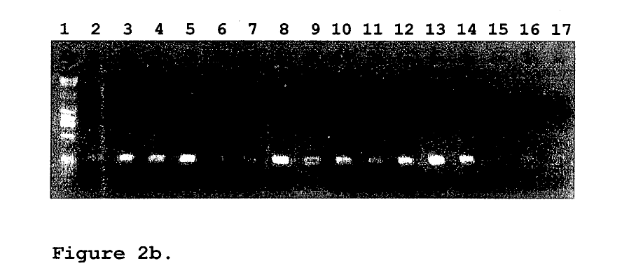

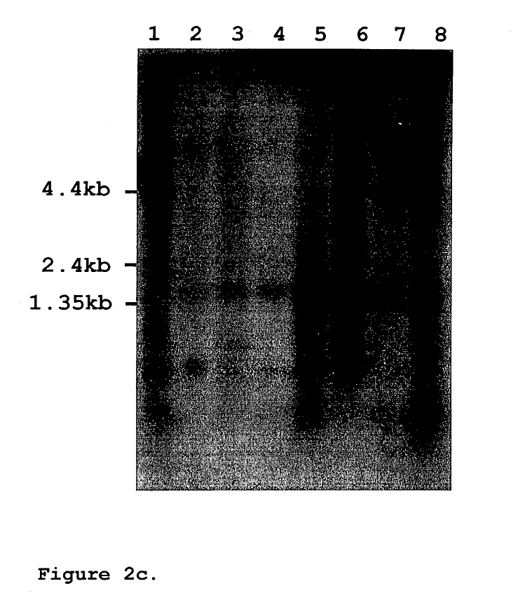

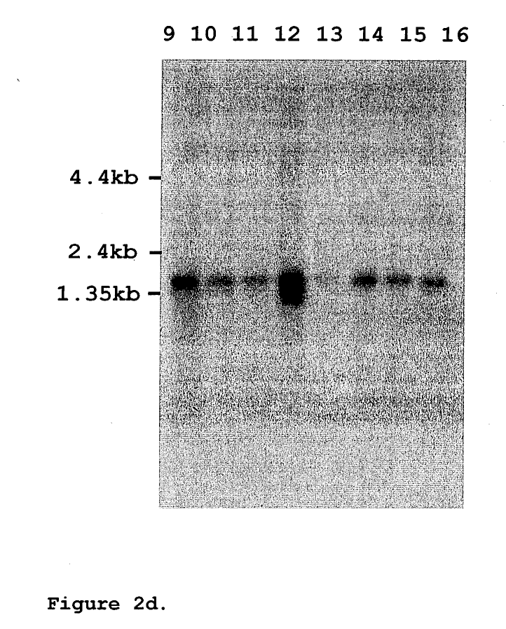

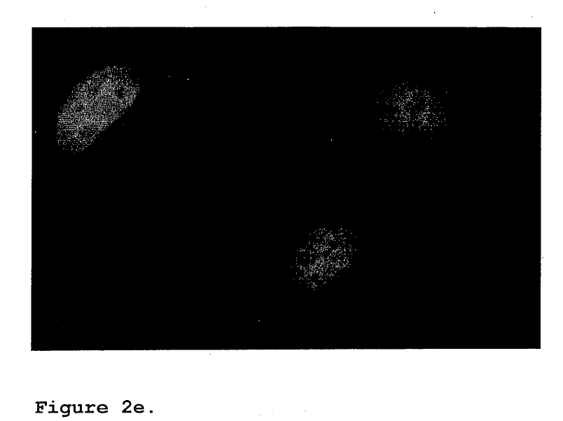

Database searches revealed several mouse ESTs homologous to the human DYXC1 mRNA. We could thus construct the mouse mDYXC1 in silico by connecting the overlapping EST clones. The mDYXC1 mRNA (SEQ ID NO: 4) encodes a 421-residue protein (SEQ ID NO: 5) that is 78% identical with the human DYXC1 (FIG. 2A). The human DYXC1 protein does not have any significant homologies to other known proteins. It has, however, three C-terminal tetratricopeptide repeat (TPR) domains, corresponding to amino acids 290-323, 324-357, and 366-399. These TPR domains are thought to mediate protein-protein interactions (14, 15).

The human DYXC1 mRNA appears to exist in several different splice forms: exons 9 and 2 can be omitted, and there is an alternative acceptor splice site in intron 2. All these arrangements, however, alter the reading frame, leading to truncated protein products. DYXC1 mRNA can be found in several tissues. It is most abundantly expressed in brain, lung, kidney and testis (FIG. 2B). Northern blot of human adult kidney tissue revealed an approximately 2 kb transcript, corresponding to the predicted size of DYXC1 mRNA (FIG. 2C).

The cellular localization of DYXC1 in transfected monkey kidney COS-1 cells was studied using immunofluorescence. The full-length DYXC1 cDNA was cloned into a mammalian expression vector containing a C-terminal V5 epitope and a polyhistidine tail. DYXC1-V5/His fusion protein showed a staining pattern similar to DAPI staining, suggesting that DYXC1 is a nuclear protein (FIG. 2E).

### Example 3

Association Analysis of DYXC1 SNPs

The present invention also describes single nucleotide polymorphism in DYXC1. In order to find polymorphisms in DYXC1, we screened the DYXC1 cDNA from 57 dyslexic individuals from 22 unrelated families with single-stranded conformation polymorphism (SSCP) analysis to characterize sequence variants. As a control, we screened 91 anonymous blood donors from Turku and Kuopio, and 15 non-dyslexic subjects from the 22 dyslexia families. In this way, we found three SNPs. Two of the SNPs (4C→T, 572G→A) were in the coding region, whereas a third one (−164C→T) resided in the 5′ untranslated region (Table 1). Both the SNPs in the coding region resulted in amino acid substitutions.

TABLE 1
Frequency of single nucleotide polymorphisms in dyslexic subjects and
controls.

| | | Amino acid | Frequency | | | | |
|---|---|---|---|---|---|---|---|
| SNP | Codon change | position | Dyslexia | n | Control | n | p-value |
| −164C → T* | — | — | 0.0517 | 58 | 0.0286 | 105 | 0.2219 |
| 4C → T | Pro → Ser | 2 | 0.0172 | 58 | 0 | 105 | 0.1259 |
| 572G → A | Gly → Glu | 191 | 0.4811 | 53 | 0.5202 | 99 | 0.7792 |
| 1249G → T | Glu → STOP | 417 | 0.1228 | 57 | 0.0545 | 101 | 0.0278 |
| 1259C → G | Ser → Cys | 420 | 0,0789 | 57 | 0,0769 | 104 | 0,9482 |

*corresponds to position 205 set forth in SEQ ID NO:2.

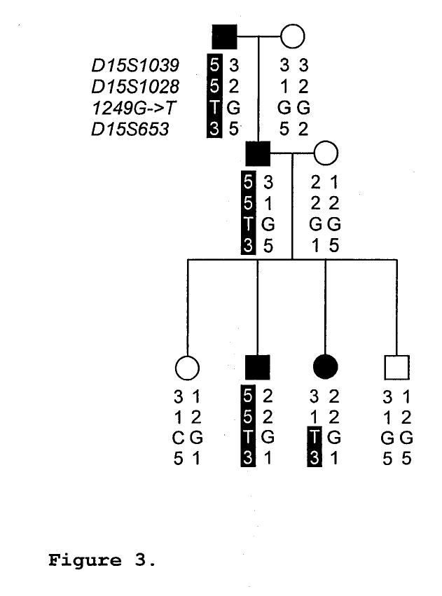

4C→T, a nonconservative substitution of proline-2 to serine-2, was found from two dyslexic individuals (a father and a son), but not in any of the control subjects. Further examination showed that this alteration did not segregate with dyslexia in the extended pedigree. Likewise, the frequency of 572G→A did not differ significantly between the two groups. The third SNP, −164C→T was found in 6 dyslexic individuals from three families and in 5 control subjects. In one three-generation family, the T allele segregated with dyslexia (FIG. 3). In the other two families, there was no consistent pattern of inheritance with dyslexia.

To search for additional SNPs, we sequenced the whole coding region of DYXC1 from an individual carrying the T allele in the family presented in FIG. 3. We found a G to T transversion at position 1249 of the DYXC1 mRNA, which results in a substitution of a glutamic acid for an ochre stop codon at amino acid position 417. The appearance of a stop codon leads to the deletion of the C-terminal tetrapeptide Glu-Leu-Lys-Ser. In the family of FIG. 3, 1249G→T was transmitted in the same chromosome as −164C→T, thus segregating with dyslexia.

Screening of all 57 dyslexic subjects for 1249G→T showed that the SNP is relatively common with a frequency of 0.123 (14/114 chromosomes). In the control group, the frequency was only 0.055 (10/174 chromosomes in blood donor samples, 1/28 chromosomes in control subjects of dyslexia families). All the control subjects were heterozygous for the SNP, whereas there was one dyslexic subject homozygous for the SNP. In conclusion, the frequency of 1249G→T is significantly (p=0.0278) higher in dyslexic individuals.

Materials and Methods

Ascertainment of Patients and Psychological Assessment

Patients were selected among families of dyslexic children from the Department of Pediatric Neurology at the Hospital for Children and Adolescents, University of Helsinki, Finland and from the Association of Learning Disabled Individuals of Helsinki (HERO), participating in the genetic study of dyslexia. The study was approved by the Ethical Committee of The Children's Castle Hospital and informed consent was obtained from the participants. Pedigrees of two families are shown in FIG. 1A and FIG. 3. The diagnosis and degree of dyslexia was determined by Finnish reading and spelling tests designed for children (16) and adults (17). The intelligence quotient (IQ) was determined by WAIS-R (18) or WISC-R (19). Reading-related neurocognitive skills (phonological awareness, rapid naming and verbal short-term memory) were assessed by neuropsychological tests (20-23).

FISH and Southern Blotting

RPCI-11 BAC clone 178D12 (Genbank accession number AC013355) was used as a probe in fluorescent in situ hybridization. The protocol for FISH has been previously described (12). 15 μg of total genomic DNA from an individual carrying the translocation and from an unrelated control person was digested with BamHI, EcoRI, HindIII, BsaAI, PstI, or SphI, and run in 0.7% agarose gel. DNA was transferred to Hybond N+ membrane (Amersham Pharmacia Biotech) with standard alkaline blotting method. PCR fragments derived from human genomic DNA were TA-cloned into pCR2.1 TOPO-TA vector (Invitrogen, Carlsbad, Calif.), and insert was removed with EcoRI digestion and gel-purified (Qiagen, Venlo, The Netherlands). α32P-labeled insert was used as a probe in Southern hybridization. Hybridization was performed overnight at 65° C. in Church buffer (0.5 M NaHPO₄, 1 mM EDTA, 7% SDS, 1% BSA), and the filter was washed in 2× SSC, 0.05% SDS at 65° C. for 1 hour. Filters were autoradiographed with a phosphoimager plate.

Cloning of DYXC1 and Sequence Analysis

Novel genes in the sequence of clone 178D12 were predicted in silico with Genscan (24) and Fgenes software. Predicted genes were confirmed by sequencing RT-PCR products. DYXC1 cDNA has been deposited in GenBank with accession number AF337549. Mouse mDYXC1 was constructed from two overlapping EST sequences (accession numbers BG242087 and AK005832) and verified by comparing it to all available mouse mDYXC1 EST sequences. cDNA sequences of mDYXC1 and hDYXC1 were aligned with ClustalX. The alignment was improved manually, and shaded with BOXSHADE. The secondary structure of the TA rich region was predicted with MFOLD (available at http://bioinfo.math.rpi.edu/˜mfold/dna/form1.cgi) with default parameters. The expression of DYXC1 was analyzed by RT-PCR from Clontech's multiple tissue cDNA panels 1 and 2. RT-PCR was performed in 25 μl volume in the following conditions: 94° C. 2′ (94° C. 1′, 68° C. 2′)×30, 1× DyNAzyme buffer with MgCl₂ (Finnzymes, Espoo, Finland), 0.2 u DyNAzyme II polymerase (Finnzymes), 15 pmol forward primer GTTGACAGAATGCTGTTCCACGTCG (SEQ ID NO: 11), 15 pmol reverse primer CAAGCTGAGGCACGAAGAGCAATGA (SEQ ID NO: 12). Promoter region of DYXC1 was predicted with TSSG and TSSW software at Baylor College of Medicine, available at http://searchlauncher.bcm.tmc.edu/seq-search/gene˜search.html, and neural network promoter prediction (NNPP) software at University of California, Berkeley, available at http://www.fruitfly.org/seq_tools/promoter.html.

SSCP Analysis

DYXC1 exons were amplified with PCR (primer sequences available from the inventors on request) and digested with suitable enzymes to obtain 100-300 bp fragments. Denaturing gel was run for 16 hours at room temperature with 5 W constant power. Gels were stained with silver according to standard protocols.

SNP Analysis

Polymorphisms −164C→T, 4C→T, and 572G→A introduced novel Tsp45I, MnlI, and MboII restriction sites, respectively. Exon-specific PCR products were digested with the appropriate enzyme and run on a 1.5% agarose gel (−164C→T, 4C→T) or on a polyacrylamide gel (572G→A), followed by silver staining. As 1249G→T had no effect on restriction sites, exon 10 was directly sequenced from all subjects. A standard one-tailed Fisher exact test was used to evaluate the statistical significance.

Cellular Localization of DYXC1

Full-length DYXC1 cDNA was cloned into pcDNA3.1/V5-6×His expression vector (Invitrogen). Monkey kidney COS-1 cell line was transfected with 3 μg of the construct, with FuGENE6 (Roche) as a transfection reagent, according to manufacturer's protocols. Cells were stained with mouse anti-V5 antibody (Invitrogen) and FITC-conjugated goat anti-mouse IgG (Sigma-Aldrich). Nuclei were stained with DAPI. The specificity of anti-V5 antibody was tested with standard western blotting methods.

References

1. Catts, H. W. (1989) Defining dyslexia as a developmental language disorder. Ann. Dyslexia, 39, 51-64.

2. Grigorenko, E. L. (2001) Developmental dyslexia: An update on genes, brains, and environments. J. Child. Psychol. Psychiatr., 42, 91-125.

3. Lyon, G. R. (1995) Toward a definition of dyslexia. Ann. Dyslexia, 45, 3-27.

4. Habib, M. (2000) The neurological basis of developmental dyslexia: an overview and working hypothesis. Brain, 123 Pt 12, 2373-99.

5. Paulesu, E., Demonet, J. F., Fazio, F., McCrory, E., Chanoine, V., Brunswick, N., Cappa, S. F., Cossu, G., Habib, M., Frith, C. D. et al. (2001) Dyslexia: cultural diversity and biological unity. Science, 291, 2165-7.

6. Wijsman, E. M., Peterson, D., Leutenegger, A. L., Thomson, J. B., Goddard, K. A., Hsu, L., Berninger, V. W. and Raskind, W. H. (2000) Segregation analysis of phenotypic components of learning disabilities. I. Nonword memory and digit span. Am. J. Hum. Genet., 67, 631-46.

7. Smith, S. D., Kimberling, W. J., Pennington, B. F. and Lubs, H. A. (1983) Specific reading disability: identification of an inherited form through linkage analysis. Science, 219, 1345-7.

8. Morris, D. W., Robinson, L., Turic, D., Duke, M., Webb, V., Milham, C., Hopkin, E., Pound, K., Fernando, S., Easton, M. et al. (2000) Family-based association mapping provides evidence for a gene for reading disability on chromosome 15q. Hum. Mol. Genet., 9, 843-8.

9. Grigorenko, E. L., Wood, F. B., Meyer, M. S., Hart, L. A., Speed, W. C., Shuster, A. and Pauls, D. L. (1997) Susceptibility loci for distinct components of developmental dyslexia on chromosomes 6 and 15. Am. J. Hum. Genet., 60, 27-39.

10. Schulte-Körne, G., Grimm, T., Nothen, M. M., Müller-Myhsok, B., Cichon, S., Vogt, I. R., Propping, P. and Remschmidt, H. (1998) Evidence for linkage of spelling disability to chromosome 15. Am. J. Hum. Genet., 63, 279-82.

11. Cardon, L. R., Smith, S. D., Fulker, D. W., Kimberling, W. J., Pennington, B. F. and DeFries, J. C. (1994) Quantitative trait locus for reading disability on chromosome 6. Science, 266, 276-9.

12. Nopola-Hemmi, J., Taipale, M., Haltia, T., Lehesjoki, A. E., Voutilainen, A. and Kere, J. (2000) Two translocations of chromosome 15q associated with dyslexia. J. Med. Genet., 37, 771-5.

13. Edelmann, L., Spiteri, E., Koren, K., Pulijaal, V., Bialer, M. G., Shanske, A., Goldberg, R. and Morrow, B. E. (2001) AT-Rich Palindromes Mediate the Constitutional t(11;22) Translocation. Am. J. Hum. Genet., 68, 1-13.

14. Ramarao, M. K., Bianchetta, M. J., Lanken, J. and Cohen, J. B. (2001) Role of rapsyn tetratricopeptide repeat and coiled-coil domains in self-association and nicotinic acetylcholine receptor clustering. J. Biol. Chem., 276, 7475-7483.

15. Blatch, G. L. and Lässle, M. (1999) The tetratricopeptide repeat: a structural motif mediating protein-protein interactions. Bioessays, 21, 932-939.

16. Häyrinen, T., Serenius-Sirve, S. and Korkman, M. (1999) Lukilasse. Lukemisen, kirjoittamisenja laskemisen seulontatestisto peruskoulun ala-asteen luokille 1-6. Reading and writing test designed for and normated in Finnish elementary school. Psykologien kustannus Oy, Helsinki.

17. Leinonen, S., Müller, K., Leppänen, P., Aro, M., Ahonen, T. and Lyytinen, H. (2001) Heterogeneity in adult dyslexic readers: Relating processing skills to the speed and accuracy of oral text reading. Read. Writ. Interdisc. J., 14, 265-296.

18. Wechsler, D. (1992) Wechsler adult intelligence scale revised (WAIS-R). Psykologien kustannus Oy and The psychological corporation USA, Helsinki.

19. Wechsler, D. (1984) Wechsler intelligence scale for children revised (WISC-R). Psykologien kustannus Oy and The psychological corporation USA, Helsinki.

20. Korkman, M., Kirk, U. and Kemp, S. (1997) NEPSY Lasten neuropsykologinen tutkim us. Revised version. Psykologien kustannus Oy, Helsinki.

21. Denckla, M. B. and Rudel, G. R. (1976) Rapid automatized naming (R.A.N.): Dyslexia differentiated from other learning disabilities. Neuropsychologia, 14, 471-479.

22. Christensen, A. -L. (1982) Lurian neuropsykologinen tutkimus. Luria's neuropsychological test. Psykologien kustannus Oy, Helsinki.

23. Wolf, M. (1986) Rapid alternating stimulus naming in the developmental dyslexias. Brain Lang., 27, 360-379.

24. Burge, C. and Karlin, S. (1997) Prediction of complete gene structures in human genomic DNA. J. Mol. Biol., 268, 78-94.

1. An isolated, purified DYXC1 nucleic acid comprising SEQ ID NO: 1 or a complement thereof; homologs and variants thereof, and fragments thereof.

2. The isolated nucleic acid according to claim 1, which is mammalian.

3. The isolated nucleic acid according to claim 2, which is human.

4. The isolated nucleic acid according to claim 1, wherein said nucleic acid hybridises under high stringency conditions to a nucleotide sequence of SEQ ID NO: 1 or a complement thereof.

5. The isolated nucleic acid according to claim 4, wherein said high stringency conditions comprise 6×NaCl/sodium citrate (SSC) at about 45° C. for a hybridisation step, followed by a wash of 2×SSC at 50° C.

6. The isolated nucleic acid according to claim 1, wherein said fragment is a primer or a probe hybridising specifically to a nucleic acid having the sequence of SEQ ID NO: 1 or a complement thereof.

7. A vector comprising the nucleic acid of claim 1.

8. A host cell comprising the vector of claim 7.

9. An isolated nucleic acid molecule encoding DYXC1 amino acid sequence of SEQ ID NO: 3.

10. An isolated nucleic acid comprising at least one single nucleotide polymorphism in any one of the following positions as defined by SEQ ID NO: 1:, 4 (C preferably to T), 572 (G preferably to A), 1249 (G preferably to T) and 1259 (C preferably to G) or as defined by SEQ ID NO: 2: position 205 (C preferably to T).

11. A method for the diagnosis of a single nucleotide polymorphism in DYXC1 gene in a human, which method comprises determining the sequence of the nucleic acid of the human at one or more of positions 4, 572, 1249 and 1259 in the DYXC1 gene as defined in SEQ ID NO: 1 and position 205 as defined in SEQ ID NO: 2 and determining the status of the human by reference to polymorphism in DYXC1 gene.

12. The method according to claim 11, wherein the nucleic acid region containing the potential single nucleotide polymorphism is amplified by polymerase chain reaction prior to determining the sequence.

13. The method according to claim 11, in which the sequence is determined by a method selected from allele specific amplification, allele specific hybridisation, SSCP, oligonucleotide ligation assay and restriction fragment length polymorphism (RFLP).

14. The method according to any one of claims 11-13 for assessing the predisposition of an individual to dyslexia mediated by the malfunction of DYXC1.

15. An allele-specific primer or probe capable of detecting a DYXC1 gene polymorphism at one or more of positions 4, 572 and 1249 in the DYXC1 gene as defined in SEQ ID NO: 1 and position 205 as defined in SEQ ID NO: 2.

16. An isolated and purified DYXC1 polypeptide comprising the amino acid sequence of SEQ ID NO: 3 or splice variants thereof.

17. Method of producing a DYXC1 polypeptide according to claim 16, said method comprising the steps of:
culturing a host cell of claim 8 comprising a polynucleotide encoding said polypeptide operably associated with a promoter sequence such that the nucleic acid sequence encoding said polypeptide is expressed; and
isolating said polypeptide from said host cell or from a growth medium in which said host cell is cultured.

18. Method of producing antibodies comprising:
immunising a mammal with the isolated and purified DYXC1 protein of claim 16 or an antigenic fragment thereof.

19. Use of the isolated and purified DYXC1 protein of claim 16 or an antigenic fragment thereof as an antigen.

20. An antibody produced by the method of claim 17.

21. The antibody of claim 20 which is labeled with a detectable label.

22. A kit for use in the diagnostics of dyslexia or in assessing the predisposition of an individual to dyslexia, comprising
a container; and in said container:
a compound, preferably labeled, capable of detecting DYXC1 gene or allelic variants thereof.

23. The kit according to claim 22, wherein said compound is a primer or probe.

24. The kit according to claim 22, wherein said compound is an antibody as defined in claim 20.

25. The kit according to claim 22 further comprising instructions for using the kit.

26. A method for identifying a mutant DYXC1 nucleotide sequence in a suspected mutant DYXC1 allele which comprises comparing the nucleotide sequence of the suspected mutant DYXC1 allele with a wild-type DYXC1 nucleotide sequence, wherein a difference between the suspected mutant and the wild-type sequence identifies a mutant DYXC1 nucleotide sequence.

27. The method according to claim 26 wherein the sequence of said suspected mutant DYXC1 allele is compared with the sequence of one or more wild-type DYXC1 gene sequences selected from the sequences set forth in SEQ ID NO: 1, SEQ ID NO: 2, SEQ ID NO: 6, SEQ ID NO: 7, SEQ ID NO: 8, SEQ ID NO: 9, SEQ ID NO: 10 and wild-type allelic variants thereof.


SEQUENCE LISTING (13 sequences)

SEQ ID NO: 1 - DNA, 1263 bp, Homo sapiens
  coding sequence for human DYXC1 (cDNA)
atgcctcttcaggttagcgattacagctggcagcagacgaagactgcggtctttctgtctctgcccctcaaaggcgtgtgcgtcagagacacggacgtgttctgcacggaaaactatctgaaggtcaactttcctccatttttatttgaggcatttctttatgctcccatagacgatgagagcagcaaagcaaagattgggaatgacaccattgtcttcaccttgtataaaaaagaagcggccatgtgggagaccctttctgtgacgggtgttgacaaagagatgatgcaaagaattagagaaaaatctattttacaagcacaagagagagcaaaagaagctacagaagcaaaagctgcagcaaagcgggaagatcaaaaatacgcactaagtgtcatgatgaagattgaagaagaagagaggaaaaaaatagaagatatgaaagaaaatgaacggataaaagccactaaagcattggaagcctggaaagaatatcaaagaaaagctgaggagcaaaaaaaaattcagagagaagagaaattatgtcaaaaagaaaagcaaattaaagaaggaagaaaaaaaataaaatataagagtcttactagaaatttggcatctagaaatcttgctccaaaagggagaaattcagaaaatatatttactgagaagttaaaggaagacagtattcctgctcctcgctctgttggcagtattaaaatcaactttacccctcgagtattcccaacagctcttcgtgaatcacaagtagcagaagaggaggagtggctacacaaacaagctgaggcacgaagagcaatgaatactgacatagctgaactttgcgatttaaaagaagaagaaaagaacccagaatggttgaaggataaaggaaacaaattgtttgcaacggaaaactatttggcagctatcaatgcatataatttagccataagactaaataataagatgccactattgtatttgaaccgggctgcttgccacctaaaactaaaaaacttacacaaggctattgaagattcttctaaggcactggaattattgatgccacctgttacagacaatgctaatgcaagaatgaaggcacatgtacgacgtggaacagcattctgtcaactagaattgtatgtagaaggcctacaggattatgaagcggcacttaagattgatccatccaacaaaattgtacaaattgatgctgagaagattcggaatgtaattcaaggaacagaactaaaatcttaa

SEQ ID NO: 2 - DNA, 1993 bp, Homo sapiens
  CDS (369)..(1628)
  human DYXC1 mRNA as cDNA
tcctctcctattatgcaaacccacaccatggatccagcagactactctggccccagcatgctgctccttgggatgatgggagaagggggaaacctgtgttttctcttggttttagggtatgggtctacttctttgaacattcgctcagatcgtaaggtaaccccaagaatcggcatcactctctggtctgggcgcctggatagtcacgcatggagtccgcgaggtgctggcgccggagcacgctgggaacgccgggctcccggccggcctgacccggaagggctggcgcatggtaaccccagcttccctagcaaccaagcaggcgcaagaagcaaccagccgcgctatccgctcccgttgctaccggaatgcctcttcaggttagcgattacagctggcagcagacgaagactgcggtctttctgtctctgcccctcaaaggcgtgtgcgtcagagacacggacgtgttctgcacggaaaactatctgaaggtcaactttcctccatttttatttgaggcatttctttatgctcccatagacgatgagagcagcaaagcaaagattgggaatgacaccattgtcttcaccttgtataaaaaagaagcggccatgtgggagaccctttctgtgacgggtgttgacaaagagatgatgcaaagaattagagaaaaatctattttacaagcacaagagagagcaaaagaagctacagaagcaaaagctgcagcaaagcgggaagatcaaaaatacgcactaagtgtcatgatgaagattgaagaagaagagaggaaaaaaatagaagatatgaaagaaaatgaacggataaaagccactaaagcattggaagcctggaaagaatatcaaagaaaagctgaggagcaaaaaaaaattcagagagaagagaaattatgtcaaaaagaaaagcaaattaaagaaggaagaaaaaaaataaaatataagagtcttactagaaatttggcatctagaaatcttgctccaaaagggagaaattcagaaaatatatttactgagaagttaaaggaagacagtattcctgctcctcgctctgttggcagtattaaaatcaactttacccctcgagtattcccaacagctcttcgtgaatcacaagtagcagaagaggaggagtggctacacaaacaagctgaggcacgaagagcaatgaatactgacatagctgaactttgcgatttaaaagaagaagaaaagaacccagaatggttgaaggataaaggaaacaaattgtttgcaacggaaaactatttggcagctatcaatgcatataatttagccataagactaaataataagatgccactattgtatttgaaccgggctgcttgccacctaaaactaaaaaacttacacaaggctattgaagattcttctaaggcactggaattattgatgccacctgttacagacaatgctaatgcaagaatgaaggcacatgtacgacgtggaacagcattctgtcaactagaattgtatgtagaaggcctacaggattatgaagcggcacttaagattgatccatccaacaaaattgtacaaattgatgctgagaagattcggaatgtaattcaaggaacagaactaaaatcttaatgactattagaagtaactaagtattgttataagttttttaaaaacaactggaggcatctttgtacatattatggccagttgtacagaatcgctttctgtttagtactttagttctgttgagggcaaaatattataaatctatagaaaataaactgtttgacttgaatcatttctgaataagtaaatctaaataagaatctattttaattccttatttcttcatattaatacatatgtatacttttttgtgttactgaattaagcttgcccttgtaacaaaatatgttttggtatagttaccaggacacttactgattaatttttaacaaggtagaattttaaaataaaagatttataaataa

SEQ ID NO: 3 - protein, 420 aa, Homo sapiens
MPLQVSDYSWQQTKTAVFLSLPLKGVCVRDTDVFCTENYLKVNFPPFLFEAFLYAPIDDESSKAKIGNDTIVFTLYKKEAAMWETLSVTGVDKEMMQRIREKSILQAQERAKEATEAKAAAKREDQKYALSVMMKIEEEERKKIEDMKENERIKATKALEAWKEYQRKAEEQKKIQREEKLCQKEKQIKEGRKKIKYKSLTRNLASRNLAPKGRNSENIFTEKLKEDSIPAPRSVGSIKINFTPRVFPTALRESQVAEEEEWLHKQAEARRAMNTDIAELCDLKEEEKNPEWLKDKGNKLFATENYLAAINAYNLAIRLNNKMPLLYLNRAACHLKLKNLHKAIEDSSKALELLMPPVTDNANARMKAHVRRGTAFCQLELYVEGLQDYEAALKIDPSNKIVQIDAEKIRNVIQGTELKS

SEQ ID NO: 4 - DNA, 1697 bp, Mus musculus
  CDS (48)..(1307)
  murine DYXC1 mRNA as cDNA
cggacgcgtgggcgacgtaaagtggctgtgcagtccctggtcccgccatgccagtgcgagtgagcgaattcagctggcagcagacgccggccacgatcttcctgtcgctgcctctgcggggcgtctgcgtgcgcgatgctgacgtattctgtggggaaagttacctgaaggttaactttcctccatttttatttgagctgtttctctatgctcccatagatgatgggaagagcaaagccaagattggaaatgacacgattcttttcacattgtataaaaaggagccagttctgtgggatagcctttctgtgccgggtgttgataaagagatgatgcagagaataagagaaaaatctatcttgcaagcacaggagaaagcaaaagaggccacagaagcaaaagctgttgccaagcgagaagaccagagatacgcactaggcgagatgatgaagattgaagaagaagagaggaaaaaactcgaagatctgaaagaaaatgaacggaaaaaggcaactagcgaattagaagcgtggaaagaatgtcaaaagaaagctgacggacaaaaaagagtccagaggaaggagaaaccgctcgagggaaagcaagctgaagagaccaaagctctaaaacctcggggtttgccccggaaggccccacccactcgcctccccacaagagggaggaattgggaaaacatatttcctgagaagttaaaggaagacagagtccctgcgcctcgctccgctggcagtattcaaatcagctttacccctcgagtgttcccaacagcacttcgggaatcccaagtcgcagaagaggaggagtggctgcataaacaagcagaagcacggagagccatgagcactgaccttcctgagttctttgacttaaaagaagaagagaggaatccagactggttgaaagacaagggaaacaaattgtttgcaacagaaaactatttggcagcggttgatgcatataatttagccatacgactgaactgtaagatcccattattgtatttgaatcgggctgcttgccacctcaaattaaaaaacctacacaaggccatcgaggactcttctaaggcactagagttattgacaccacctgttgctgacaatgccaatgcaagaatgaaggcacacgtccgacgagggacagcgttctgtcaactagaattgtatgttgaaggcttgcaagattatgaagctgcacttaagattgacccagccaacacagttgtacagaacgatgcagagaagattcggaatataattcaagggacggcactgaagtctcgtgactagaggcagctaggcacaggagtctctgcaagtactgtagctctgtacagtaccgggctagtgagcctttggaatggcagtgcctgacgaatcatctccaaggggaaaatggcctagctctcactgcctttgatcccagtaccgggaaggcagaggtaggcaagtctctatgagcttgaggccagccagagctcgacagcatgcaaaacacaaaagaaagccagagaacaggctgtttgagtcctcaatcttggcacagggattggctgggctctgatgttgctgttgaaattgccaggatactactgaggaatctacagaatagaatttccacataaaagattgtgcatataaaatattcttattatattaaatatagtatttcattcc

SEQ ID NO: 5 - protein, 420 aa, Mus musculus
MPVRVSEFSWQQTPATIFLSLPLRGVCVRDADVFCGESYLKVNFPPFLFELFLYAPIDDGKSKAKIGNDTILFTLYKKEPVLWDSLSVPGVDKEMMQRIREKSILQAQEKAKEATEAKAVAKREDQRYALGEMMKIEEEERKKLEDLKENERKKATSELEAWKECQKKADGQKRVQRKEKPLEGKQAEETKALKPRGLPRKAPPTRLPTRGRNWENIFPEKLKEDRVPAPRSAGSIQISFTPRVFPTALRESQVAEEEEWLHKQAEARRAMSTDLPEFFDLKEEERNPDWLKDKGNKLFATENYLAAVDAYNLAIRLNCKIPLLYLNRAACHLKLKNLHKAIEDSSKALELLTPPVADNANARMKAHVRRGTAFCQLELYVEGLQDYEAALKIDPANTVVQNDAEKIRNIIQGTALKSRD

SEQ ID NO: 6 - DNA, 50000 bp, Homo sapiens
  human DYXC1 chromosomal gene region, nucleotides 1-50000
aagcttttgctttccttgatcatgtactgttgagaaagcaatatttggaaacattgttgcatttttaacattatctttaagaattaaaaggcacatacctttgatgccataattctggttctaggagctacttgtaaatctctctaaaggcatttatacatggatttttattgcagcaacagcagaggcttggaaacaacttaaacattgatcaatatggaggggactagtttaatgaatcatggtgtagtcacactgtagaatactactcagctattacaaaataaagaagttctaaatagagtgatatggaatgatctccaagtaagaaagcaaggtacactgtgtgtactgtgtgtactttgtctcatttgtgtcctccccgccaaaaaaataataataatatactttaatatatctagattatctttggaaaggtaggccagaaactagtaatagtggtggcctctgagacagggaaccagttactaaagattttagagtgagagagagagacttgctttttcactgaaagaataataaatatgggagcagggatgaatggctatttctggctcaaactatgggaaagacttttttgaaacagataatgataataaaagtattgctcttgtctaacgaaaatagaaaacattaacaggtaaatataatagatattactatgagatataaagtaacttcacttacaagtgggaaatattacctgaatttttttttttttaggtggtgatagtggactggatgggttaggaggaccaggtgtacaactaggaagcccagataagaaaaagcgcaaggcaaatacacaggtaaactgacattttctttaataaaagtttttctttgctagtatggtcaggctgatgaggtaaatttgaacttggtcataagttttgttttctattcaagtgattgtacttttaatcattcattgtactttcaatcaatcattccagtgatggtactttcttttttttttttttttttttttttttttttttttttttttttaggtggattcttgctctgttgccagactggagtgagtgcagtggggtgatctcagctcactgcaacctccacctcccaggttgaagcgattctcctgcctcagcctcccaagtagctggaattacaggcacgcgccaccatgcccagctaatttttgtatttttagtagagacgggctttcaccatgttgatcaggatggtctcaatctcttgacctcgtgatccacccgcctcggcctcccaaagtgctaggattacaggtgtgagccaccacgcctcgctgtatttttttttgtttaagtctgtaactagtggctattgtcagactataggcatagactagatactgcaaaaaggaagtgtgggcaaattaaaatgcttgttcttaggctgggcatggtggctcatgcctataatcccagccctttgggaggccaaggtgggtggattacttgaggccaggagttcgagaccagactggccaacatggggaaagcccttctctactaaaaatataaaaattagccgggtgtggtggcacactcctgtaatcccagctattgcggaggctgaggcatgagaattgcttgaacctggaaggtagaggttgcaatgagctgagatcacgccactgcactccagccttggtgacagcgtgagactctgtctcaaaaaaaaattaaaattaaaatgcttgttcttaaatctaatttaaaggtttttaaagggcatgaatgcctgcagttctattaaaagatttcaaaatatatagtaacaattttaacctgaaataattgttcaactataatagtgatttagtattatgtgcatatcacgaagcaatcatgatggaggcctttttttgttataacttgtggttgtaacaagcaattatatatacaggctctcactgttttattctttataaatttgaagcactctttctatgataaaggctactagtaagtatgtgtgtacagattactgatctttttatcattttgaccaccatctgttaaacttgcagtgtgactaaaactggcagtttattttttcagtctgaggcccagaacttaatgttttggaaatattttaattgtatctccctgaagttaagctaggggaaaaacttagattctgattgttgttatttaactttatgactgtttggtgtatgagaggtattttaggttagactgctttagtatttaagtgaaagtttctagagttataggtaagcatattaaatgaaaattaggataatgaagcttcttttgaaagttaactacttttggtttttgaagttacaatggatttgagctcagaaaatcatactttattttcttttggaaatgaaataaaactaaatatttttatcagcaagtataattaaatataaacattagtcaaaattgaaaggtcaagtgaagataataaatgtttgttaacactgcaagagaaatttgactaatactaggttcacaggtattaaggtaacaggacaattttaataatgtatttgatcgaaaacatgtattttactttacattctcatttcagggaccttctttccctccattgtctgagtatgctccaccaccgaatccaaactctgaccatctagtggctgctaatccatttgatgacaactataatactatttcctataaaccactaccttcgtcaaatccatatcttggccctggttatcctggctttggaggctatagtacattcagaatgccacctcacgttcccccaagaatgtcttccccatactgtggtccttactcactcaggaaccagccacacccatttcctcagaatcctctgggcatgggttttaatcgacctcatgcttttaactttgggccacatgataattcaagtttcggtaatccatcttataataatgcactaagtcagaatgtcaacatgcctaatcaacattttagacaaaatcctgctgaaaatttcagtcaaattcctccacagaatgctagccaagtttctaaccccgatttggcatctaattttgttcctggaaataattcaaattttacttctccgttagaatctaatcattcttttattcctcccccaaacacttttggtcaagcaaaagcaccacccccaaaacaagactttactcaaggagcaaccaaaaacactaatcaaaattcctctgctcatccacctcacttgaatatggatgacacagtgaatcagagtaatattgaattaaaaaatgttaatcgaaacaatgcagtaaatcaggagaacagccgttcaagtagcactgaagccacaaacaataaccctgcaaatgggacgcagaataagccacgacaaccaagaggtgcagcagatgcctgcaccacagaaaaaagcaataaatcctctcttcacccaaaccgtcatggccattcgtcttctgacccagtgtatccttgtggaatttgtacaaacgaggtgaacgatgatcaggatgccatcttatgtgaggcctcttgtcagaaatggtttcatcggatctgtactggaatgactgaaacagcttatggcctcttaactgcagaagcatctgcagtatggggctgtgatacctgtatggctgacaaagatgtccagttaatgcgtactagagaaacttttggtccatctgcagtgggcagtgatgcttaatcaaaggcattaactaaagtgggtttattttcctgtgcattgcagaagttcattgacacaggattttaatgttttacattatttttttaaatgcatacacaaaaacattatttacttagtttttattaatcacttcatcactagagcaaattttttattgtctttgcttgctgtcttaaatgagaataatgtatatgggggtgctttagaaacataaataaatgttagttgttgttatttatcacaaacataaaagcctcttgaaacttcaaaataatatataacaaatgtaggcaagttttaacttttaaaagttagaatcattgaaaaacgtgtatttcagtgctgaactttggttgtaccatattaaagatttagttcaaaaagtttatcaaggatccacttaacacgtcttcacaacataatatatgtaactgtaacatgggggaaagcatacatttttatggattaaaaaaataatagtatgacaccagattcctcctccactagggtctttggaatcaatgaatttcatatttcaagtgagttttcaggatatctgtttgtacagatacgagacagatcttagcatatagtaaattctcagtaagtattgttgattaacttgtggatggtaccacatgaaattgctaatattagatcagttgaaaattgactgcaattagtaaagttggtataacgtacgttcccagttgtgctatttagaattatgatagtattatgctcctacttttctttgatattgagttgtcttgttattgtttattgttttccagagaaatactatagtatccacagaagaattgtgaatacttttttataattttgaactgggacatattgcagatgaggaaggtagaaacatccatttggccttagagggctctgctagtaaaagaactactcagtggtggtttgaaaaatcttgttctggggatctgaatttcaaaaatgaaccctaggggagatcaaaatccattttaaacaaagatcaattaagagccaaagtgaaatctaacttgcaaacaacaatttgaagccttgcagcagacttaaggaattcccgctctgactggggaaaagtataaaaatggacctgagagtaccctcttgagggatgatcaagaatggagacagttggagttgtgctcattaagattcaaaacagcagaaatccatttttggagcagattataacctgtggccaaaaaaaaaaaatgtgtgctgtttgacagctaatggcaatttatacaatcattggaggctggtaaagctccctaatatgttgccaccattatcattgcaaccaaaaaaattctgaagattgtgtggtgatctacgaaagagataaataattactgttatttaaacccaagtgaaacaatgacggcagcagcatattatcaagaaatttaaattatgcatgaaaatgtgatttaaaaacaatgtcctttgggtaacacacaagaacctacactttcacagacaaggactcgacctcacatgtcacaagctactaaagcaaagttaagtgacttgggctatgaaattttgtcatgtccattttagtcactggacctttattccatggactactgtctatcatttggagctttttctaagaaacaatgcattccccaatcgagatgaagtaactggaagatgttaacaatttatacagcctaacaaatgatgaattttataaaagttagacctttaatattctgttggaagaaaactattcatgctcctgatgcatattttgattgaataaaacttatttttttaaatacattgttttaattttgacctacaaaaacagcaatttcatatggtacaaggtaatacaaggttttctccatttacccttgactagttagaggtgagtacaaaatattttcagtagttgtaaaatatttatctttacaattttaggaaacaaaatgatataaactatacttctaggtactcctttgtgtgaaaactttaccagttgaagcaggttaaaatgcagtattttgggctgacaggcatttgatggtgttcagtagtgtacttgaagatctcattttacttggattttttattatacatatttctctaaatgtttaaggacctagttatgttgctcctgaaagtttaatgagtgctgatttttaataaatgatgcactgtatgctgtatgcctagttgtagcatcagatcatgatttattattaccctgaatagagctagatgatagatattttgagtagaactttccagtgttctcaggagttaacactttacatggtagtacttgagttgtgatcattactaaaatcaattatgcagtttcttaaaccctttatctctttaagtttcagtagttagctgtttttgtgaaaatgaaataacttctcccaagctagtctaaaggtatgtgttatgcaatataattcattttctatttccacatagttgtttttaaaaacctacaaacaggtgaatttttattcaggtgtttaggtcttcaaagcaccttacttaaataagaatctgtaaatattcaaaagaaaaactcacggttcattctgatagtgaatcatagccaaactgtaatcatagctgatgaatagcgttcatgtgtttgaattaattttcctgctagttctacatttgtctctacataatgctaagtcaaaagttatatagatgcttttggatggaataagtgatgaactgtaagaatagccagatgataatttgtctgaagaaggctattatacaaactgaaatggcttcttaggataaatgataagctgccttcaactttaaaaatatgcagtgatggaaaaaaacacaggtggaattgtaccttttttggtataatccctttccttcactgaaagtcctcatcttacacacacacacacacacacacaccccccacagtttctccaacatccttgagttttgcttaaattcaacctggctaactatgagcatctaggtttactcaccaaagctaggttattgacagaaaggtgggtttttttttgttttttttgttttgttttgttttgtttttgttttttgccagtcttgctctgttgcccaggctggagtgcagtgatgagatctcggctcactgcaacgtctgccttcctggttcaagtgattctcctgcctcagcctcctgagtagctgggattacagtcgcccaccatcacacctggctaatttttgtatttttagtagagacgggttcaccatgttggccaggctggtgtcaaactcctgacctcaagtgatctgcccgcctcagcctcccaaagtgctgggattacaggtgtaagccaccacgcctggccagacagaaaattttaaaaagcatcagttcctttctcagaacttgacttgctcatgcactagagaatttgtggtccacatgcttctaggtgttactcgattcactaatactgaaacagctcagcttgtgaaagataaattgttcttcagacagagtgcttagaattagatttgtgatcagtgttttccttttgtgtgaaatactttagagtactgctataattgttattcaaattagaaggttatatacataaaactaaagattttcttgtactagtgcttattcttcataaacacagtgtaaagaaatctacttttttggtggtttgttttagactcccaactctgcctagtggcagaaaaacctcttaatagtgtgggtcatcataaaatctgccaactttaatccaaataagtatcaagagtcttcattcaatattctttcaaacttgtttgtctcgaacaaattagagttttagcacttattgttcaatgtactatagtaattgtatttaatagtaagaaaatatataagcaatgattttttaaaagggagttctcaagagttttggatttctttctgccgtgtatagaggtttcttatgcatggacatttgtgctgttttatatctttttcaattaaaaaaaaatttaaaggcatcaggctccctaagttcatttgtaaatagtggcttggaactctacacaatagggaagctcctaaaggctcatttagaccataatctagttaatagtatcatgctgaactctcagttttctttaggaaaccaatcaatcagtgtaggagggagaaagaagcagaggtcttgtctttttcctgatgtgattaggaggttaagttctttggtgtgggtttctttgcatttgcccccttaccatgctaaactctggtgaaatgtataagcagccttggcttttcctctcctgggtgctctgtgcttcaacattttatatctttcctcccatcagaaatcctgtgtctttgtgtggcagtagtagttggctagactttaagagaaaagcattttctactaaggtatgaatgaagatagtgggaaaagtgtcattaaatttcttttttaaaaaagtatgtttattaagaatgaatctttaatatctaacatttcagacaatatgaccgcatttcagataccatgggaagcagtacatttagtgaattagctggtactgtctgagggtacttccagtttcctttaggctgttgtgacttttcacagcttcctctcctctaattctgaacttagagattttcttctcatttctaatttttagtagcttattgttccatccagtagagcattataaaagtcacatctgcatcttccaacttatggtacagtgatgggaatttggaagcacccctggagcaaagaggctagggatagcattaacatctcccaagacaagtggtataggggaaatgaaggaaagactgtggccatgtatatgccaggcgttaatacatcagagctgcctgtatttaatttaaaacttttgatgaaactcagtggataagcatctttgtcatattttgtcaataatacattacttgcaattatattaaaacttattctaaatacctgtacctttcttgttctgtagatgtgtagatgatgttgcttgtttgtcaaaatattatcttttataaaagaaagtcctgcttcttatcatgatgtatgtgacttaaatgattgtttcatattaaaactatttcttccttatgtactgtttacatatacctctttttgggatattagttcagtacttttatacaaatctgagtgtgttccacattggtgacatgtatttttgcagtaactttttgttttgttcttagaacatgtctaaagtaacacatgcactagtgctgtggtctgtggcaaaattactgtaattcaaattggaaaaaaaaatgtttccagactttgtccaacatttattcctatggcaaaggaaattactatgtaatactaattatttcttatgctggtatgttttaagctactctatgaagtatgtgaaacatcaatatttatggattaatagatggcctgatgtctttcaaatgggagaggagtaaattttagtgttaccaactcatgacacttgtaatacaataaatcttaaaatttttattttccacttacaggtatactttactaaataattaccttaaatggcagtgcatattaggattttttagcacatactactaaaattatatagtaacttttaacttgtgctgatatttcccaaccaaaaaagacatcattcacttcatcataaccaaaataagtagtactgtaatatttatcttcatttatttagacatgattaagataccgtaagactcctctaatttacctggaatttttagccattgattcaaggacttgaatgctgtttacttggtactacctaatgaaaggatttggtaataaaggttataatcacgattttaaagaaaaatgtttcatcttgaaggcaaatttttaagctttttatttagaggtgtttacttaagaatagtaatttaaaagcattcttattttgtcattaagacaagtacccaaaagaattgtaataagtaatacttggccaatgcaaatttgtacatttgaaatcatacacttgaaagaaaaaaatgaaggcttgtgagtgcttaaactgcatatctttttaaaaattcaaattttagacatatcagtttaaattatccttaataaggttttactatgtcagtgatgtatattacaaaatgtctacctatgttatgttaatgatatctctatactgcatcatattcggcatggttatggtatattttgtacttttgattgtggactattgataggattgatatagcaataaatctgattcagacttaatattttttaaaacgctagataccatactgtatcataatcctattatatttgacttgaatgcccaaaattttcatagttctatattttatgttaaaaatttagtattacatgttagggatttatttttgtctgctattttcacatacattcttttagaactgagtctgtttctatttagatcatagacgtctaaagaagtattattaattcatgaactgctttgtggaaaaatttaaataaagtatgaaatgactagattaccccaaattgtttatttttttttttggttaatgaccaggttacaattctcagacaaatgaaattggcttgataactgattgaatatagtagataatggaaagtttgactctttaagggcttccaagtgttaaatacattctcaattctgaattacttcatagtaactacttaaataatggtaagccaattctcctttttctttttgagatatttaacacaatttgcgtcttctgatcccattcccttcttccccaaaaaccatgctagaatttaaaattggaagagtttaatttgtcatctacattttagtttttttattctatagataagtaagaagacaagtagacgttctgctgtcaagacagtccatgttattgttggccattcttttcagggataatgcttaatgttatttgtgcgttgtataaggaatgttgtttatcagtatatagacctaccattagtagaaatgatgtatggcaggctttcttgaataatatgtattgtaagtttgtttcctatagtgttattatggttcagattcttttaaataaagctagtttttaaagagaaaaataactttttaaagcgtttgactttcatcaggacaagtaggtgtgagaatcttggctttgggtgggagatgtccagtataacatcaagcagggaatagcctacaatttatcagtgtaattttatactaaggagagaaccagtatttttagtcaggccttgctgtatacatattttttctgtgaagtctattcagaaaactgagtcctggatttccttaagaaattgtgagcatgacatatttactttgattttaacagtttgtgacttcagttctgttagtaaaaattatttggagaagagttttacaacttcagcagcttaatgacccctggggcagagacaccactgtagcaaatattaagaacaaacttgtggctgggtgcagtgactcacgcctgtaattccagctactcgggaggctgcggcacgggaatcacttgaatccaggaagcagaggttgcagtgacagtgagctgagattgtgccgctgcactccagcctgggcgacggagcgagactcacgagactctgtctcaaaaaaaaaaaaaaaaaagaaaagaaaaaaagaacaaactttcttgaatgataagacctctagtttccttgaaacaatcatcagttgccttgagtctgttacataaaaagaggctgaataaaataatgtgtcaactgatgaaagcttgacattataggtaagaagtgataagatgaaatctcaagataaaatttgaatgcgtgacatttcaaatttaataggaaaaaataaaaatatgtgtatctcgctgaaggagagagaagatatatatagagagaattcttatcaaataacattgatgctctaagttactccctctgttttaaacatatagcagtgataaatatgaaaagataaaattgaactcaaaaccatgctaaatccaaggacaagtaatattgtacagaaagaaagcatgagccatggtgaagcagtagcctgcctgactctgagttctcaagcagaaacttgcagtaggaaattctaatctgacaaggtaatgaggtctaaacatcttaaaacatgagatgcaataacagtgctaaaagggaaatatatagctttagattatttttaagtccaaagtatgatgagctaagttgatctcaagagactaagaaaacaacaacaacaaaaaacttgaaagtagaaggaaggaaataataagaaataaaataggaaaacactagaaggaataagaagaccataaataggttatttaaacaataaagatgaggcccagagcggtggctcatgcctgtaattccacactttgggaggccaaggtgggaggatcacttaagcccaggagttcaagatcaacctgggcaacataaggagactctgtctacaaaaaatttttttaattagctaagtgtggtggtgcacacctgtggtctcagctgctcaggaggctgaggtgggagtattgcttgagcccaggagttgaggctatagtgggccttgatatgccgctgcacttcagtctgagcaagagaacaagaccttgtctaaaaaaaaaaaaaaaactgagcctctgacaagactgattttttttttttttttttttttttttttttttgagatggaaacttgttctatcacccaggctagagtgcagtggcacgatcttggctcgctgcaatctccacctcccagggtcaagcaattctcgtgcctcagcctcctgagtagccgggattacaggcgcatgccaccatacctagctaatttttgtatttttagtaaagacggggtttcaccatgttggctaggctggtcttgaactcctgacctcaagtgatccgcctgcctcagcctcccaaagtgctgagattacaggcatgagccactgtgcccggccaaaactgattttttttttttttttaatggtaggcagaaatatcaggaatgaaaatggaggcaagcatagcactgctttaagccggtgctgagctgggatttctctccgctgctgtaccacttgtgaaaggaagctaaaaactgaaacctaccttcaactgatacctggggctctaatttataggaagatgcagggctaagggggtagaaatacgcagtttttaaaatgtacagagttggtaactggtttacaatatgaagaaagaattgaagcagtgaattagcatgtaacacctcgctgaattggggtccctaagacacaagttggaggcaacccaaaattgctagaaagaaaaggatgaacccagaggaaagaaagataaatgaagggaacaaaaaagacactgcgaatcaaaatttctgaacacatgagatctgaagacaggaaaaccagcaaatcaataccttttgaaatttcagtccactcaagatgaaaataatggaataaactgaaactttaaataagtttgcttaggactttgaaaagtaagctaaacaataaaatagaaatgatgaaacccaaataagtagacattaatccattcaatattttactgtcttaatatgttcaggcgctcttctggatgctagaactatagcagtaaatcaaacagacaaaaatcactattcatgaggtttttattctaatgatacagacagtaaaagggtaagtgcaattaagaaatgaaggggctaggtgcagtggctcacacctgtaataggaaaataataggaaaataataggaaaaagaaaaatactaatcaaagaaatatagcaggccgggtgcggtggctcacgcctgtaatcccagcactttgggaggccaaggtggacggatcacctgaggtcaagggtttgagagcagcctgaccaacatagtgaaaccccgtctctactaaaaatacaaaaattagccgggggtggtggcatgcacctgtaatcctaactactcgggaggctgaggcaggagaatcgcttgaacctgggatgcggaggttgcagtgagccgagatcttgctactgcactccagcctgggcaacagagcaagactccatctcaaaaaaaaaaaaaaaaaagatagaaaaaagtataccagaaaaatactaatcaaagaaatatagcaggctgggcaccttgttcacatgagccagcagcagcagcagtagtggcatagtggcagcatggtgggggcacacccatccgctgcagcaggatgtagcaggtgctggggtgcctgcctttgtgcaggcattcaccacagtggcgaaggagtacagcttccaggggcccctgctggtgactatgtgtgcggttggctggtggtagtgttagaatggggtgcaggcaggcgcaatctgtatgtgttctctgtgcaccacaggcaggggtggtcactcaagtcgggggaggttccgctggtctctgtgcctagtttcattcccatggcagtgttggtgcaagggcatgccagcaaagcaatgtggggggttgctgtgggccaggcaaaacagcgtgtagggagggagtgggtggcctggtgtgtggctgtgggggctgtcctgctgtagttctcttgatggtcaggcatggtccaccagcacagaaactatgatgtgagcccccaggacacccaaggctgtttgacaggcaggctcaatcaggcctgggccccaggaggggccagcagaccaacggatgctcatgtcagaccggcctgtctgatgggcaagaccacgtaacagagttcaggtccaacacttcctctagggctaaagtctcctatgggagcatgttgagcctagagagatgggttatcctggccatgcttcgctacagatgctcccacaccaaaccctctgggctcctcttcagcagacgtgctgcccctaccagttctctgagcagctatccctgccaactcaagggtccttggtggtcagggggtctcctgccaggattccagaggcacgcagtgagagtgggttgctccttaccagttcaacttacctgttccctcagagtttctgagggccaggaatgagtcctggtgcatagtagccccatgcagggctcccagcttcctcctgcttcagcctagcttctgtgtcttccctttgttcattctggtgccttccctctgaagatctgttaggagtataacagtcatctcagtccctcagtggcagctgttccatctggttgcatctagttggccaccttgcccccattttcacccaagaaaacattttataaatatttttaaaaccccaccaaaatgataaaataatcatggacatgtatgattgcaaaagagcctcaacatatataaagcagaagatacaatcacagagaattatagggagaaataagcagcaattgcttaagattttaaaacaaatttctttcagaaaatgatagtccaagcatcaaattttttaaaattatttttaatatatatttttgtccacttaggctgctataacagaatactattgactaggtagtatgcagactatgttgtctgaccaaaagataatgaaaaaaaactatgtatgtttagaaaatgaaagtaaactcttgacctcatgttccgcctgcctcggattgagaccatcctggcaaacatggtgaaaccccgtctctactaaaaatataaaagttagccaggtgtggtggcaggcacctgtagtcccagctactcgggaggctgaggcaggagaatcacttgaatctaggaggcagacgttgcagtgagccaagatcatgccactgtactccagcctggtgacagagcgagattccatcaaaaaaaaaaaaaaaaaaaaaaaaggtaaactctttagtacttaattcatcagaaggaaagttatgtaacatttataccttaaagacaacttaaatactacacatcaaaaccacaggggtaggtttaggatatagaaaactgcaaagaacctcatttccactctaaccatgagaaaaagctggataatccacaaaaccatgactttttctaaaaccatcatagagctaattttaaaggcaatcaagtaatctgaatagcaaagagaaacaagattctccaaggagagaaagcacattaacgatttcacatttagcaaagtggaagagaaaggtggccatcatatcaatgggaaagaagaaatcaactaaattgttttaaaggccatatatgggttagtgtgtcagtttgtcataggtgggggcctcagacaccaagtgagtttgcactcccttgtaggccttcactggatgctcaagaaaatgactgggggcaggagagaagactgaagaaatccccctcacccaccacccacagcagtggttcaaatgttcatgtgattagtagattcttggcgacaggcataaagtgcaattttctaaccccttctctcctacaaatcaaaagtcttaaaacattggggaggatcatcaaactcttttgtccctggacacaggcaaagaattattgtttttaggggagggccagaagaaaagcttgtttgcttcttggtaacagataggaaactatcccaataccagaacaaaggcaaagatccattgctgctggaatggtaagggaggaagggtaaaggaaaaaactctttgccactgtaggaaggacagggaatttcttgggcaaaggaggcttcattgatatcaagcagattatgagaggaagtcctggaagggggtaggaaactgtgtccctagaccaatcacaaagataaggcagagctggtggccatgagaggaaggaaaagaataatgaggaagtcccactactgagacacaggtcctgtgcctctcccctaagcacaagaccttagtagcacacctgtagaggaatctgaagtttgtgttgcactaaagacagctctaccaacaacaaaacccaaacccagctcagccaaggcaaacaaaagctgagaatttgttgcaagcagacctgtgctaccagaaagattaaaggatgttcttcacgtcgtatgttctcataagtgggagctaagctatgaggatgtgaagccataataatgacacatggactttggggactcggggaaagggtggtaagggggtcaaggataaaagactacaaattgggttcagtgtatgctgctcgggtgatgggcgcaccaaaatctcgcaaatcaccactgaagaacttactcatgtaaccaaataccacctgttccccaaaaacctatggaaataaaacacacacacacacacgaaaagaaataaataattcttcttttgagaagtctgaaaaaaaggatgttcttcaggcagaaggattatattgcagagaaacttgtatccacaccaaaaaatgaatagtgttggaagtagtaaaaatgaaggtaaatataaaagatatttcttcttgtttgtaattgctttgacccacatattatatatatatgtctatatatagagagagagactgtctatatatatatagtctattgatttctgaaaacattgccaagataattcaatggagaaagggtacactttttaaacacatgatacgagacaattggacaaccatatgtgaaaaaatgagcctggacctatacttggtatcatataaaaatttaactcaaaatgaaacacagatctacatttaagacctaaaaccataacatttctagaagaaaatcaaaaagctttgtgtgttaggttagaaaactatttcttagttaagactcaaacaaatattttaaatgttgttaaactgagctttatcacaaaacttctattatttaaaagacattgtgaaggatgaaaagacaaacatacaataaaataaaataattgaaaagcacatatctcataaaggattcttgtatccagaatacataacacacatgtagcagacatatcaccgaagagtgaaacaaataagcacatgaaaagatgcttaacatcactggtcaccagacaaatgcaaattaaaactgtagtgaggtacaaatagacaactactagatttaataaaatgttttaaaagcccacaaaactactgatacccagtcctggtgaatatgcagagtaatggaaacttttatacattgctggtagaaatgtaaaatttgtacttttgaagtagtttagtagtttcttataacattagtaaacttaaacctatcatatggcccagcaattatatttttaagtatttactcctgaatgtttatagcagttttattcataattaccaaaaactggaaacaaccaaaatgtctaccaactatagaatggacaaacgactctttgtacatgtatatcatataatacttagtaaaaaaaaagaatgcatgaaacacaacagcacagacggatctccaaagcattgagctaaataagaagaaaacgaaagggttcatactacatgatttccttatataacattctgaaaaagacaaaacaataagaaaagaattcagttcagtggttgccagggatcggggctgggaggatgtaactgagtagcttaggttcaaaatgcattttgagacttttttttcttccttttcttccttttagccttgaaacataccttaatactctttgtttccctgccttcccaccagacactcttgtgcactggtagtttatctaattatgtgcccagaagttgcaggggctaatcttgagataaaccaggcatggaaacccaggtgtgaaattctagagattacctcagagcagttagtcaacagcctcaccattgttgagatgatgccagcctgcactccaagtggaccatgactcaggataaccagtggaacaagacacacagacctcgtacccagcaccattcctgcatgccttccattccaagttcccctctttaaacccctgtccacggcctaaagtttgaaatagtctgttaagagcacgagtctggacattccccatctgctagcgtttgaataaaagctgtttccctttggtcacacctcatttctcatactttgacttctaacagtgagcagttgaacttgagtcagttacaggaggaggaaattgactgcaaagaaatacgagaaaactttggtgagatggaaatattttatatcttggttgtggttgaggttacatagctgtttatattgatcaaaactctagaacaatatatctcaaacgagtggattttactgcatgtaaattatacctcagtaaacctaactttaaaattctaagatacagctctcgcaatatttggaaggccaatttgcagtcttaaatgaacctataaaaaccaaggagcttttaaaaaaatgaattatgatttaactcaagagtttgaagaataacaagtagaagaatggaaacaaatgtaagagcaaaagttaatgaaatgaaaaatgggagaagatcaacaaaaataaaggttatttttactagtctttcaaaaaactaataaacatgtaataagagagatgggacagaaagagattatacaaacaagcaatattaggaataaaaacatgattatcttagataaagtagaaaatgctataagtagctttatgtcaatggaggctaagactagttgcaggaagaataagaaataggatattggtgtgattgcagattatctgtctcccacaaaacactaatcaattacaaagaagaaaattatgactttacagtgaagaaacttggcagacaccaatatgacaggtgatgaccaatgttaccagtggtgggaacagtcaaccgcatatgtccgtactatggctgatactcttacaagggcatagtttcatttccatgatattcctgccatgaatgcatgacctgagtgtgatcataaggaaacatccagctcaaggatcactctacaaaaggtaaggcctgtactctttcaaagactaaggagctttttgagattggagaaaactgaagagacttgataactgtattagtccattttcacgctgctgataaagacatacccgagactgggaagaaaaagagttttaattggacttacagttccacaggtttaactctgtgcacaaactagtcagtccactgaaaaggataacagacaggtgagtttgtgtcctttgattgcttaggatctgttgctaatttgctgaactgaagctgcaagagccttgtgtttttgagcatctagtgggaaaagtctttacctctggttgtgtgagtttgagatatattcctacctcagcgggggtggagggggagggtataagtaaaataaattagaagactctttaaagagtaccgttatgattgttttggagcaatgtcaccactctctcctcagggattttaggcaatgtctggagacattttgggctgttacaatttgggggtgcttttggcatctaatgggtagagatttggtgtgctgccacacagctcacaatgctcaggaccgccctgcatgacaaaacaattatctggcttaaaatgacaatcgttttagagaaactgaaaagcacttatataaatctaatattctcaacatttcctcaacataaattgaatgtctaatgctttaaatgtgatagcctgcttaaaaaaaaaaagtccttctcacagaagctgctagctcaaaagcactttaaaaaaaagcacttagaaaaaaaaaaaaaaacaagaaatcaagttcatgcaattaaactagactgataatttgggtttaaaaaataactgtatcaggccgtgtgtagtggctcatgcctgtactctcagcactttggggactgagttgggtgggtctcttgaggccaggagttcaagaccagcctggccagcatggtgacaccccgtctctactaaaaaaaaaaaacaaaaaaacaaaaaaacattagctgggtgtggtggcacatgcctttaattccagctactactggggaggctgaagcacgagaaatgcttgaacctgggagacagagtttgcagtgagccaagattgcaccactgcagtccagcctgagagacagagtgagactgtttccaggaaaaaaaaaaaaaaaaaaaaaaacaccaaaaaagtcgtatatttttctgattatatcagtgtgaagaaaaaatacaagtatacaagttttattctttttaaaaatatggattgtttacataaaggtaattttgtttttgttccttttttttcaggttcaggaatacatgtgcaggtttgctgtataggtaaattgcgtgtcatggagatttggtatacagattattttatcactcaggtaataagcatagcgccgggtgggtagtttgttgatcctcaccttccttccgcccttcactcttaagcaggccctagtgtctcttgtttccttcattgtgtctatatgcacttaatgtttagctcccacttgtaagtgagaacatgcagtatttggttttctgttcctgtgttagttcacttagaataatggccttcagctccatccatgttgctgcaaaaaacatgatcttgttcttttttatggctgcgtagtaatccatggtgtatatgtactacattttctttatccagttcaccattcgtgagcatctaggttgattccatgtctttgctgttgtgaatagtgctgtgatggacatatgtgtgcatgtgtcattatggtagaattatttatattcctttgggtatatacacaataatggtattgctgggttgaatggtgattctgctttgtgttctttgagaaatcaccaaactgctttccacagtggctgaaataagttacattcccaccaacagtgtataagtgttcccttttctctgcaaccttgccagcatctattattttttgactttttaataatagccattcttactggtgtgagatgttatctcattgtgggtttgatttgcatttcgaagagaattgctactttgaatttctgacatttcttatacagatttaatctgataagctaatattattcctacgtaatatttaggatgaggaacagtgaataattttgtttaagtggattggtttataattctgacaaacttttttttgttatttgtttgttttggtttgttttcttcttagagacaggtctcgctctgttgcccaggctggagcacagtggtgctatcatagctcaccgtaacctcaaactcctgggtataagtgatcatcctatctcagcctcctgagcagctaggactacaggtccatgccaccatgtctggctaatttatttgttttgtaaagatgggggtctcactttgatgcccatgctggtctcaaactcctggcctcaagcaattatctggccttaccctcccaaagtgctggaattgcaggcatgagccactgctcctggcctctgacaaagtttttaatatcaacagtacattaggggcctccagacctgggcccccagcacagcttcccaggccctgcctgatcacttcaatccgttgcagctctgcatttctctgaggaggaaatcccagagaccatccacaggctgtctgccattgccactgcagtggtacccacctttgctgccctctggctggggaaggaacaaagagcctaattgctttgctggcagctcccatatgtggaggagcccagtctctctttcctataatccctcaacccactgcttttcaccaggtagggcccccagcttgggcctgtagcacagcctgcagtaggtttgtgtttttctagggtggagcttctgcaggcaactgacagcccctctgttactgtagcagtatctgcccttcctgccatcaggctggggaaggaaaaaatagcctgagtgctttactcacacctccagcacgccatagccgccctgcagagaagaagtcagactcttccccatgggccctccatccccctgctattcaccaagcagggatcccagacttgagcccacagtgcagcctcctcaccttgggctgattgttctgattggcagtggccctgcgtttctctggggcagagacccaagagacgagtgaaaggccctctaccattgccactgccaaggttcctgcccctgctgcccccaagctggggagggaacaaaaagcctgagcttgccacaaagctgtgttgtgcaggcctggagtgccaagccgagatctgtggccagcactcaagtggaagaggagcccacattcagagagcactgacagggagcatggctgcgtatgtgaggaaatgaaagaggagccacgtggctaagagctaccggccctatggttagcgccatctactggatcacagctcaaacttcaacatcaaaaatacttctgttagtagaccctgtgaaaccaaggacaaaaatccagccccaaataaacaccctgcacaaagcctcagccctctgaaaacatccagaaacaaatccaactggctatattgaaattacaccacagttaaggaacatcagcacacagatgagaaagaaccagtacaagaactcaggcaactctaaaagccatagcgtttgcttacctccaaatgactgcgctagctccccagcaatagttcttaaccagaatgaaatggctgaaatgacagacatagaattcagaatctggatggcaatgaagatcattgagattcagtagagagctgaaatccattccaaggaatctaagaaatccagtaaaatgattcaagagatgaaagatgaaatagccatatagggaaccaaactgagctgatagagctgaaaaactcactacaagaatcttataatacaatcagaagtattaacagtataataaaccaagctgaagaaacaatctcagagcttgacaatcagttcttcaaattattcaatcagacaaaaataaggaatttttttgaataagcaaaacctctgagtaatatgggattatgtaaagagaccaaatcaatgacccattgacatccctgaaagagaagaagagagagaaagcagcttggaaaacatatttgggaatattaaccatgaaaatttccccaaccttactagaaaggttgacattcaaattcaggaaatttagagaagccctgcaaggtactattcaaggctatcaccctgaagacacatagtcattagattcttcaaggtcaacatgaaagaaaaaatgttaaaggcagctagagagaaggggcaagtcaccaacaaagggaaccccatcaggctaacaatggacctttcaacagaaatcctacaagccagaaaaacttggggctaatagtcagcatccttaaagaaaagaaattccaaccaagaatttcatatccagccaaatgaagcttcataagtgaaggagaaataaaatccttttcagacaagcaaatgctaagggaattcattaccaccagacttgccttacgagaggtttttaagggagtgctaaacagggaaatgaaagaccattactgaccaccacaaaaagacactcaagcatattgaccattgaaactaaagcaaatatgcaatcaagtcttcaagtctacataacaaccacctaacaatacaatgatgtgatcaaatcagcacacatcaatactaactttgaatgtaaaggggctaaaaatcccacttaaagggcatggagtggcaagctggataaagaagcaagacccagtggtatgctatcatcaagagacccatctcacatgcaaggacacccacaggctcaaagtaaagggatgaagaaagatctctcaagtaaacagaaaacaaaagagtaggagctgcaattcttatttcagacaaaacagactttaaatgaagaatgatcaaaaaggacaaagaaggcattacacagtgataaagagctcagttcaacaagaagatttaactagactaaatatatattaaccgaacactggagcactagattcataagacaacttcttacagacttacaaagagacttagataaccacacaataatagtgaaagacttcaatgcccactgacagtattggacagatcactgaggcagaataactgacaaagatatttgggatctaaacttgacacttgaccaaatagacctaacagacatctacagaacacttcacccaacaaaagcagaatatacattcttctcatctgcacatggcatacactctgaaattgaccacatgctgagccatgaagcaattctcaactaaataaaaaaaaatcataccaaccacattctcagaccaccatgcaataaaaatagaaatttttatttctattattttatttctattattatttatttaccaagaagatctctcaaaaccacacaattaaatggaaattaaataatctgctctggaatgacttttgggtaaacaataaaattaaggcagaaactaagaaattatttgaaatgaatgaaaatgaagatacaaaataccagaatctctaggacatagctaaagcaatgttaagaggaaagtttaggctgggcgcagtggctcacacctgtgatcccagcagcactttgggaggccaaggcaggcgaatcacaatgtcaagagattgagaccatcctggccaacaacgtgaaactccgtctctactaaaaatacaaaaattagctgggcgtggtggcaagtgcctgtagtcccaactactagggaggctgaggcaggagaatcacttgaacccgggaggcggagcttgcagtgagccgagatcatgccactgcactccagcctggtgacaattaaatggaacctcacctgctcgggaggctgaggttggagaatggcatgaacccaggagacagaggttgcagtgagccaagatcgcgccactgcactccagcctgggtgacagagcaagactccgtctcaaaaaaaaaaaaaaaaaaaaaaaattaaataagtgagaaagatctcaaattaacaacctaactttacacctagagaaactagaagaaaaacacaaagccagccccaacgctggcagaagaaaagaaataatcaaaagcagagctgaactgaacaaaatggagatgagaaaagccatacaaaagatcaacaaaaccaaaagctgcttttccgaaagaataagattgatagactgttagctaaactaataaaaaaagagagaagatccaaataaacgcaatcagaaatgacaaagaggacatgaacaccaaccccatagaaatacaaaaaccatcagaaactattatgaacatctctatgcacacaagctagaaaacctacagcaaatgggtaaattccaggaaacatacaacctccaaagattaaaccagaaagaaattgaaaccccaaaaagatcaataatgagttctgaaactgaatcagtaatgaaaatcctacccgaccctggaccacatggattcacaactgcattctacgaaatgtataaagcagagctggtaccaatcctactgaaactattccaaaaaattgaggaggagggtatccttcctaactcattctatgaggccagcatcattgtgatacaaaaacctggcagagacacaacaaaaaggagaactttaggtcaatatctctggtaaagataggtgcaaaaatcttctacaaaatactaacaaaccaagtccagtagcatatcaaaaagcgaatccaccacaaccaagtaggctttttccctgggatgcaaggttggttcaacacaggtaaattaataaatgtgatttatcacataaacagaactaaaaacaaaactaaatgaccatctcaataaagaagaaaaggcattcagtaaaactcaacacactttcatgttaaaaaccctcaacaaaccaggcgttgaagatacattcctcaaaataataagagccatctatgacaaacccacagtcaacatcatattgaatgggcaaaagctgtaagtattccccttgagaactggaacaagacaaggatgcctgctcttatcactcctattcagtacagtactggagctcctagatagagcaatcaggcaagagaaagaaataaaagacatcaaaataagaagagaggaagctaaactatctctcttcacagaaaatatgattctatacctagaaaaccctacagtctctgcccaaaatctcctaatctgataaacaacctcaaagtctcccagatctgataaacaacttcagcaaagtttcaggataaaaaaaatcgatgtacaaaattcaggagcatttctacacaccaataatgtcccagctgagagccaaattaaaaaaaaaaatctcattcacaataaccacaaaaagaataaaacacctaggaatacatgtattagtccgttttcatgctgctgataaagacatacctgagactgggcaatttgcaaaagaaagaggtttaattggacttagagttccatgtggctggggaaagctcacaatcacggtagaaagcaaggaggagcaagtcacatcttacgtggatggtggcaggcaaagagaggagcacttgtgcatggaaactcctgtttttataatcatcagatctcgtgagacttactctctctcatgagaacagcatggaaaatacctgcccccatgattcaattacctccctccaggtccctctcacaacacgtgggaattcaagatgagatttgggtggggacacagccaaaccatatcaatacatctaaccagggaagtgaaagatttctataatgagaattacaaaacagtgctgaaagaaatcagagacaaaacaaacaaacggaaaaacatcccatgctcatggacaggaagaatcaatattgttaaaatggtcatactgctcaaagcaatttacagattcgatgctgttcctatcaaactcctcacattttccacagaattagaaaaaaactattttaagattcatttggaaccaaaaaagagcctgtatagccaaagcaatcctaagcaaaaagaacaaagccagagacatcacactacctgacttcaaactatactacaaggcttccgtaacaaaaacaggatggtgctagtacagaaacagacatataaagcaatggaacaagttagaggacccacagataaagtcacgcatctgcaaccatatgatcttcagcaaagctgatgctaacaagcagtgggaaaagaactcactagtcaaaaaatggtgctaggataactggctagccatatacagaatgttgaaactggaaccctatgtttcaccatgtacaaaaatgaactcaacatggattaaagactgaagtgtaaaacctaaaagtataaaaaccctagaagaaaacctaggaaatactcttctgggcatcagacctgacaaagacttcatgatgagaattccaaaagcaattgcaacaaaagcaaagactgacaatagggacctaattaaactaaagagcttctacacagcaaaagaaactatcaacagagttaacaggcaacctagagaatggaagaaaatctttgcaaactatgcatctgacaaaagtctaatattcagcatctataaagaacttagatcaacaagcaaaaatcaaatgaccccattaaaaaaatggcaaaggacatgaacagatacttctcaaaagaagacatacacaccaataagcatatgaataaatgctcaatatcactaatgattagagaaatgcaaatcaaaaccacaatgagataaccatctcacactagtcagaatggctactaataaaaagtcaaaaaataacagatgctggtgaggttgtggagaaaagcaaatgcttatacactgttggtgggaatgtaaattagttcagccactgtggaaagcagtctggagatttcttaaggaacttaaaatagagctaccatttgacccagcaatcccattactgggtatatactcagaggaatataaatcattctgccaagatcataaagacacatgcataaagacacatgcatgcatatgttcattgcagtactattcacaatagcaaagacatggaatcaacctaaatgcccatcaatggtagactggataaagaaaaagtggtccatatataccatggaacattatgcagccataaaaaaggacaagatcgtgtcctttgtagcaacatgggtggggttggaagtcattatcctaagcgagttaacataggtacagaaaaccaaataccacatgttctcactgataagtgggagccaaacattcactttaagattctgaactatattcaattaagtcataaataatctttgaatgctttgataatttcttttttttttttttttttgagatggagtctcgctctgtcgcccaggctggagtgcagtggcacaatctcggctcactgcaagctccgcctcgcgggttcacaccattctcctgcctcagcctcccgagtagctgggactacaggcacccgccaccacacctggccaatttttttgtatttttagtagagatgggatttcaccgtgttagccaggatggtctcggtctcctgacctcgtgatccgcctgcctcggcctcccaaagtgctaggattacaggtgtgagccatcgtgcccggcctgctttgataatttctaagtaatatagaatccccagaccaatctcagttgttatttcgtacacatgcttttggattttatttttataagttatagagtggttctttgagtcttactaatgagcgtgttaattttttccacttcaatgtgtgaaagggatgtgtgtggacatatgaggttgcattatgtgaacttatgagcttttctgggctactaaatgcttcttatatgtaacaaatagttctcaattgccacttcttagtagtttccatgaagtaaaagtagcttctttgattacagttctaaataatacaagtggtagagattattgcaggtgcagtgtgacagagaagtatgaagagaacactattatttaaagcaaagtgtttggtgtagaatgttaaagaagatagtaaaggatgaaacttggatggtgaggaaaattaaagtcatgcaaacagtgaactttatttgccttggtttatcataactacagataatgagcctgaaaagaagcaataaaatttattacaactgttagtaagttttgcccaatttttatgagttttacgagtagaaatttaagataaagtaaaaatgtaaagtttttaactgcctaaagtttaggttaatacaaaattataatttattttttcccttatgttaaaatgataaattggtttcagagccttcagtctgttgttaaccacaggcaggttatttttttaccttcaatttaatctactcagataatgaaactttatttttttgagacagggtcttgctctgtcacccaggctggagtgcagtggtgccaacatggatcattgcagccttccattcctgtactcaagcaatcctcccacctaagctccctgagtagctgggactacaggtgtgagccaccacgcctgagtaatttttttttttttttagagacattatctcactatgttgctcaggctggtttcaaactactgagctcaagcgtcaagtgctctgcccacctcagtttcccaaagtgctagggttataggtgtgagccactgtgcccagcctgcaattttatatctcacaagaaaagttttctgattttgtcatggccttgattacagtacttaaaaacaaaaacaaaaaacaaagtactcatgctgaaaaagctgaagttccttacaattgtgttatcttttaggatttttttcaccctatatttttcttttctctagtgtcaactcctgtgccaaccttgatttataggtagtcaagttaaaataattcattgttctcagatttctatgattttttcttaatcaagtgacaaattctgctatgataactttttgattatgcttttccaagatcgaaccctaaattatattttcaaaaattcaagatgtcttttagacctaactgtttttgagttttcccaagaggcccctaaaaatcatgaagatttattctttttttttttttttttgtgagatagagcctcactccattgccaggctagagtgcaatggcgtgatcttgccactagatttattctttactcaaaaaggaatggtaaaaataattgaaattgcctgatatatttcatatttctgaagtagcagaacattattgtaagaactgcatgggaaaagctgtcaaatttaaagttcaaccatttctagttcaatttttaggtaaatagtaccaatataaacatgtttcagagattatgcacttagcataatttttcaatgtcttcactatccatagtatgttctttcctttgaggaaaatattgaccaaatcctttttaaacagttatatattgtataccagtagagagtttttctcttctccattttcatttagcatcaggcattcagtattatcatcggcaggttatttctgcttttctctcatgctcacctgaagcctttgctataagctgtaggttagaaagggtcttggctgggcgcagtggctcacagcactttgggaggccaagatgagaggatcgcttgaggccaggagttcaagaccagcctggtcaacagagcgagatcccatctctcagaaagaaaaaaaaagaaagaaagaagaaaaaaagaaagaaacaaaggaaggaaggaaggaaaagaaagaaggaaggaaggaaaagaaagaaagaaggaaggaaagaaaggaaggaaggaaaagaaagaaagaaggaaagaaagagagagagagagagagaatgaaagaaagagagaaagaaagaaagaaaagaaaggaaaagaaaaaaagaaagaaagattgcctcaatctaaggaggaaacacttagggcattgcagaagactatagaattgatatcctcagctattgatacgtcaagaaattaagacagaggcagtgaaaagtggatggatctgagagatatttaggactctgaggttgattggatttgtggcacctatctgatatgatttggatgttgtcccctccaaatctcatgttgaaatggaatcctcagtgttgaaattagggcctgctgggaggtgtttggatcatgggggaggatcccttgtgaatagcttagcaccgtccctttggtgatgagcgaattcttgctctgagttcacccaagatctggttgtttaaaagtctgtggcacctcctcctgtctctcttgctcccactctcactatgggacatgctagctttctgttgccttccaccatgattgtaagctttctgaggcctcaccagaagcagatggcaacactacgcttcttgtaaagcctgcagaaccattagccaattaaacatgttttctttataaattatccatttgtatttatagcatgcaaaaatgaccgaacacactatcataggtataattatcaaattcaaaagcattcagttcgtgaatattaaaagcttcagttttgttctcactcagatagaaaaaaccatttcatgtctgtcaggatgtgaatcagaaaaatgactgtggctgggcatggtggctcgtgcttgtaattccaacacattgggaggctgaagtgggaagattgcttaaagccaagagttcaaaaccagcctgggaaaaatataaagaccctgtctctacagaaaagttaaaaaaaaaaatgcttctggtaaagtccaagaagaaaacgatagatctgctcaaatgtctgaataaagtattgataatatgagggctttacacaaaatcataattaatcctttcaataccctacatatagatattattgcttgcattttacagataaggaaactgaggcttggggggagaaccttactcagggtcatacagctaataaaagtagaattggctgagtgtagtggtgcacgcctgaaattccagcactttgggaggctgaggtgggagggaggcttctgcccaggttgcagtgagctatgatgacagtgccactggattcctgcctgggtgacagagtgggaccctgtcactaaaaaaaaaaataacgataattaaaaaaaaaaaaaagaaaagtagactaggcatactaacccaatttgtccagttacaaacacttgattagaggaccaaattttgatgctaaggaattaaaaaaaaaaaaaaggattttaccctggaaagatgttaacattcccaacattcctgaccttagatcctgagccccctggtttaaaggccgcctctctgaaccacttccagttgcactttctccaagtgtaaggaagctgttcacctgcaacctgctcacccttctacatcatggtttggctacctcgaatccctgcccaacagccccaggctacttcccggagtcttttctctggctagtccttctgagggtggcctgtgagtcttgagtgtacatctctatgtccaatgacaggctgttagctctgctatgttcctgcatgaaaccccaaggagactgagaattccaaatgtaaatttggccttccaggtcattatgaatgcatattcagactatgtaaggaaatagagtttattttattaaatactttcttagcttaatttataactaccgtgtaacatttgattcacatgtataagggaggagcccacccctcatattgtcttatgccctaggctgcgtcttgctcctgtacacggatgaccttctgctggaacatgccatggcagttgggtgtgcgaaggggacggatgccctgcttcagcacctggagaactgtgtgtataaggtgtccaagaagaaagctcagatctgcagacagcaggtacactacctgggattcgctattcggaaaggggagcgcagcctggggtcagaaagaaagcgggtcatctgcagcctacaggaacctaaaaccagaaggcaagtaagggaattcctgggagctgtggggttttgcagattatggattccaaactttgctgtactggtcaagtctttgtatggagttacaaaggggggcgaccgggagtcttttgaatggggacctctacaacagcaagcctttcgtgagttaaaggaaaaccttatgtcggccccagtcctaggactaccagatttgacaaagccctttacactgtatgtgtcagaaagagataaaatggcagttggagttttaacccagactgtggggccctggccaaggccagtagcctatctctcaaaacaactagatggggtttctaaaggctgggccccatgtctaagggccctggcagcaacagccctgttagcacaagaaggagataaactaacccttgggcaaaacctgaatataaaggccccccgtgctgtggtaactttgatgaataccaaaggacagcatcagctaacaaacgctagattaaccaagtaccaaagctggctgtgtgaaaattcccacataaccactgaagtctgtaacaccctaaatcccaccaccctgctcccagtatgagagcccagtcgagcataactgtgtagaagtgttggactcagtttgttctagcagacctgactttcaggaccagccatgggcatcagtagactgggagttatacgtggatgggagcagcttcatcaacccacaaggagaaagacatgcaggatatgtggtggtaactttggatgctatcattgaagccaaactgttgccacagggacgtcagcccagaaggctgagctcactgctttaacttgggctctagaactcagtgaaggtaagactgtaaacatcgacactgactctcgatatgcctttctaaccctccaagtgcgtggagcattatataaggaaaagggccggttaaactctgggggaaaggacataaaatatcaacaagaaattctacaattattagaggcagtgtggaaacctcagagagtggcagtcatgcactgcaggggacaccagcgaacctccacttcagtggccttaggaaactcccgagctgactcagaagctcaaaaagcagcatctaccccttactgggcatcggtagcagcctccttactccctcaaacacctgacctggtacctacctattctaaggaagaaaaagaatgtttcaacacagaagggggcaagtaataaaagaaggatggatcagactgccagatgggagggaagctgtgccgcagttgctgggagccacaatcatactggccatgcatgaaaccactcatctaggtcaagagtcacttgaaaagttgttaggccggtacttctacacttgccagcccttgccaaagcagtagcacaacggtgcgttacttgcagacagcagaacgcaaggcaaggccccactgttctgcccggcctataagcttacggagtagctccttttgaggatcttcaggtggaattcacagaaatgccaaaatgtagaggtaacaagtatttgctggttcttgtgtgtacttactctgggtgggcggagtcttccccaacacgaaccgaaaaggcctacgaagtaacccttgtgcttctccgagaccttattcctaggtttggactgcccttatgaatcagctcagataacgggccggcgtttgtggctgacttggtacggaagacagcaaaggcattaggaatcacttggaagctgcgtgccgcctactgacctcagagtttcggaaaggtggagcgaattaatcggactatcaaaaatggtttagggaaagcacgtcaggaaacaggattaaaatggatacaggcccttcctatggtattgtttaaaattagatgcaccccttctaagaaaacaggatactccccttatgaaatactgtatcataggcctcctactatactacggggacttccaggcactccccgagagttaggtgacattgagttacagcgacagctacaggctttgggaaaaattacacagacaatctcgacttgggtaaatgagaggtgtcctgtcagcttgttctccccagttcatcctttctttccagttgatcgtgtgtggatcaaggactggaacgtagcccctttgcggccatggtggaaaggacctcagaccgtcatcctgaccacccccacggctgtaaaggtagaaggaatcccagcctggatccaccacagccatgtgaaactggcagccgctgaaacctgggaggcaaaaccgagcccggacaacccctgcgaagtgactctgaggaggccgacaaaccctgctccagtcacacccggaagctgactggtctacgcacggccgaagcatgaggagaatcatcgtgggactcattttccttataatttggacttgtatagtaaaaacttccactgattttccccgcatggaggactgctctcagtgtatacatcaggttactgaggtaaggcaacaagttaaaacaatctttctgttctatagttactgtgaatgcctaggaactttaaaaggaacatgtttatatactgacactcagtacaaggtatgtagcctaggaaacgaccggccagctgtgtgttatgacccctctgagcttatccaacacgaaactgaaaaggcctacgaagtaacccgtgtgcttccctgagaccttattcctaggtttggactgcccttacgaattggcttagataatgggccggcgtttgtggctgacggtttttgaaataagattaagggctgaagactagtggggacgcgtaaatgatacaagtaaagtaatagccagaacagaagagaaaggggtgcccaaacacataatcttgaaatttgatgcctgtgctgtcattagtggcagtaaattaggaaagggatgtggctcttttgggaaaaaggcgatatgactgaaaataagtacatttgtcatgaattaggactgtgtggaaatgaatgtgaacactggtcttgtgtcatttaggccacttggataaaaaaatgaaaaggatccagtccacttcagaaaggaaaaagtggcccttcctgtactaagggacaatgtaaccccttagagctagcaacaaccaatccccttgatccttgctggaaaagaggggagcatgtgaccttaggaatcgatggggcaggactggatccttgagtaaatatcttagttcgaggagaagtttacaaacgctctgctcagccagtgtttcaaactttctaagatgaactaaatgtgccagtaccagaaattccaggaaaaacaagaaatttgtttttgcaattagccaagcatgtagcccagtctctcagtgtcacttcatgttatgtatgtggaggaactgtgatgggagatcaatggccatgggaagcccaagaattagtacctacagacccagttcccaatgaattcccagctcaaaagagtcaccttgataatttctggaccctgaagcctcaattattggacaatattgcatagctagagaaggaaaataattcactcaccccttaggacaacttagttgtctgggacagaaactgtataatggtaccacaaaaacagtcacttggtggagttcaaatcacacagaaaggaatccatttagtaaattcccaaggttgcaaaccatgtggacccacctggagtcccactaggactggacagcccccactggattatactggctatgtgggcatagagcttacgctaaattacctggccagtgggcaggtagttgtattactggcactattaaaccatctttcttcctactgtccataaagacaggcgaactcctgggcttccctgtctatgcttcccatgaaaaaagaagcatagctataggaaattggaaagataatgaatggcccccagagagaatcatacaatattatggacctgctacttggggacgagatggttcatggggatactagacccccatttacatgctcaaccaaatcatatggttacaagctgtcttagaaataatcactaatatgggctgggcgcggtggctcacacctgtaatcccagcactttgggaggccaacgcaggtggatcacgaggtcaggagattgagaccatcctggctaacatggtgaaaccccgtctctactaacaatacaaaaaattagccaggggtggtggcgggtgcctgtggttccagctacttgggaggctgaggcaggagaatggcgtgaacctgggaggcagagcttgcagcgagccgagattgtgccactgcactccagcctgggcgacagagtgagactccatctcaaaaaaaaaaaaaaaaagaaaagaaagaaagaatcactaataaaaccgacagagccttgactattctggcctgtcaagagactcagatgagaaatgctatctatcaaaatagattggctcttgactacttgctagcagctgaaggaggggtctgtgggaaatttaaccttactaattgctgtctacacgtagatgatcaagggcaagtagttgaagacatagtcagagatatgataaaactgtcacatgtgcccatgcaagtgtggcatggatttgatcctggggccatgtttggaaaatggttcccagtgttaggaggatttaaaactcttataataggagttatattagtaataggaacctgcttactgctcccttgtttgctacctgtacttcttcaaatgataaaaagcttcatcgctaccttagttcaccaaaatgcttcagaacaactgtactatatgaatcactatcgatctgtcttgcaagaatacatgggtagtgaggatgaaagtgagaactcccactaattgactgaggttctcaaagtgggggaataagggaggagaccacccctcatattgtcttatgcccaatttctgcctccaaagaaagaagtaaaaattaaaaggcagaaatggaatccacaggcagatagcccagcatcacgccctgggcctggtagttaagaatcaacccctgacctaactgctcgtgttatctatagatttcagacattgtatggaaaaacattgtgaaaatccctgtcctgttttattccgttctgatgaccggtgcatgcagctcccagtcacgtacccactgcttgcttaattgatcacgaccctctcacgcggaccccgttagagttgtaagcccttaaaagggacaggaattgctcactcggggagctcggtttttggaaacgtgagtctgctgatgcttccagctgaataaagccctttccttccacaactcggtgtcgtctgaggggttcttgtctgtggttcgtcccgctacacgtacagggtaatggtttaatatttcatgaataatattaattgtatttcaaaattaatgtggttaaatatactgatatactttaatacattttttcaacagtttaatgttttggcatagataataaatacaaataggaatttgcatttatctaatgacaaaatgttaacattttaaactctactcagttataaactaaactcaattttaaaatgtttaaaaaatgtgtggatgacacaaaaacaaaccttcatgtttgtttatattctccccttattaatcctctttaaggattaattaagcctacaaaattctactttaataatagttgttctccttaagagtcccttaaaacttttttttttaattaaataactaggccaggtgtggtggctcacacctgtaatcccagcactttgggaggctgaggtgggtagatcccctgaggtcaggcattcaagaccagcctggccaacatggtgaaaccccgtctctactaaaaatacaaaaaaattagccaggcatggtggcgcatgcctgtactcccagttactagggaagctgaggcaggagaatcacttgaacccaggaggtggaggtttcagtgagccaggatcacaccactgcactccagtctgggcaatggagcgagactccgttttgaaaaaaaaaaaatgaaataagtggtattgtgccaaaacaagtctaataggaaaaacaatcaagctcataccctataaatatatatctaagatattaggacccaaaagaaaaggaaaattatgcagggtcacttcacattctacatgacctctacctagatattctcatttaattttaaatcacattatcatttccgaaagcccaaatttaaaatagtaaaataactaatgcatttaacacctagtgcctagagtttggtctgtaaagctattctccactaaatagaactaaaagttcttggagaatttcaattcctagagaaaaaaaaagaaaagaaaaacctttaaggtggacctaagtgatcttgctactagaatgagggatttttttaaatgccttttaaagttccaaagtggtgttaagaaaacatagaaagcaaccttttccaatggcaaaatttcgaattttgcacatcaaaagtaaataaaattataggtaattaattttaggttacgaatagtaaatcatacttaatttttgctccataatgtatgcttacactctctaagaacctcaggatatgaaataaggaaataagtatgcttgtgtactatagagattgttttgaaatttttactttaaacttgtaatattttttacaaacaaaataatttatggatttacatacataaactattaataataaaaggaataagtataaatctatcactgaaaattacagagagaggaaaaaaataacctggtgatatggtttggctgtgtcaccacccaaatctcatcttgaattgtagttcccataatcccaacgtggggacccagtgggtgataaccgaatcatgggggcagttacctccatgctgctcttgtggtagtgggtgagttctcatgagatctgatgattttataaggggcttccccctactttgctctgcacttctccttgctgcggtcatgtgaagaaggacgtgtttgctttcccttccaccaagattgtaagattcctgaggcctccccagccttgcagaattgtgagtcaattaaacctctttcctttataacccagtcttgggtatgtctttattagcagcgtgagaacagactaatacacctgtcagtctccttcatgctttctgattgcacgctttaatgcaatgaccagaatcaaacagaaacgaccttgggtgttttctctggattttcctgggcaaccagccagtgggacctggaccttctggctcacagttggaggggctacacctgaactcagcttttcatgggatggacagtttcttccaacacaagttccaacaactttctgtttgacccaaggtctccagatactggataatgggctttttcaaaaaatacctatcgggccgggcgcagtggctcacgtctgtaatcccagcactttgggaggccgaggggggcgcatcacgaggtcaggagattaagaccatcctggctaacacagtgaaaccccgtctctactaaaaatacaaaaaattagccgggcatggtggcgggcgcctgtagtcccagctactggggaggctgaggcagaagaatggcgtgaacccgggaggtggagcttgcagtgagctgagatcgtgccactgcactccagcctgggcgacagagtgagagtctgtctcagacaaacaaacaaacaaataaatgcctgtaaacagactgcctggtgctctagtagtttccttactgacaaaattctttttgtgtgtgccctataaataggagagcaacaagaggatgggattcctctcctattatgcaaacccacaccatggatccagcagactactctggccccagcatgctgctccttgggatgatgggagaagggggaaacctgtgttttctcttggttttgtaagtttcccctttccccagtaaaactcatcccttaatctagtggttctcaaactttggtgtgtatcaaaattatctggtaggctggataaaacacagattgctgtgccccacttccagaacttctggttcaatagatctagaaggagactgggaatttgcatttctaacaggtccccagatgatggtaatattgctggtggtggtggggaggaccacattctgagaacctctgtatttaattttatttgtatttatttattttatttattgttttaatttaatttttgagtccctgttactcaggctagagtgcagtggcataatcatagtccactgtaacctgaactcctggcctcaagtgacactcccaaagtgctgagattacaggcgtgagacacaatgtgcatcccgggccttcgttttaattcatacagtgtgactatgtggtaatcagttctgttggttaccaaaaaggctcccacttgtatgaacttcacataagagttaaccactctcctttctagtgcctatctttacagacgacccctgacttatgatggtttctctttgatttcctaactttacagtgatgcaaaagctatatgcattcagtagaaaccatgctgtgagtacccatacaaccattctgtttttcatttttaatacagtattcaataaattacagggtatattagacactttattacaccttattatgttagatgactttgcccaactgtaggctaatgtaagtgttctgagcatgtttaaggtagggtagcctaagctatggtgtttggtaggttaggtgttttacatgtatttttgacataggatattttcaacttatgatgggtttatcaggaggtaaccacattgtaagtagagaagtgtttgtatagttttaccacatacctaggaatccttatctagtggattcagtttccgttgttcaaaagttttataaaggccaggcatggtagctcatgcctgtaatcccagcactttgggagatggaagtgaacggatcacctgaggtcagaagttcgagaccagcctggccaacatggtgaaaccccacctctaataaaaatacaaaaaattagccaggcttggtggtgggtgcctgtaatcccagctactcaggaggctgagacaggagaatcgcttgaacctgggaggcagagattgcagtgagccaagatgccccattgcactccagcctgggtgacaagagtgaaactccatcaaaaaaagaaaaaagaaaaataaaaaagtaaaagttgtataaaatagcattatttcttgaggatattatttgacttgcttttttcactcatccatccaatgtatagctatcattcactcatttttattgttgtataatattctgttttgtgtatatacctgttgaaagacatttggcttattttcaggtttttttctatgaagttgctatgaacacctataaatgtttcctagtgtataggaccaagactttttctagggcagtaattctcagatgggggaaattttgctattcccacccacccctgaaacccagaagacatttggcaatgtctggagacacttttattattagaactcaggagtgttactagaattaagagggtggatgctgctaaacatcttaaaatgcacagagcagcctaaattatacaaaatgatccagcctataatgttaatagtgtggaggctgaaaaaccctgttgtaggacagtggttcacaaagcatcagcatcacctaggaacttgctagaaatctctaagatagatacataggagtggaattgctggttataaaataagcaggtattcacattaataagattatgccaatttgcccattttattgtggggttgtgtttctttttctctatatattttgtttatatgttctctttgtttattattgagaaagagggcaaaaagcagcccctgatattcagaagctgacctggcactcacagcttggccataatattattctgttgaacattaacaatttaaatctcatagaataccaagctgagacaaaagtactctgagatcacaataaaatgagacaaggccacttcatagcattatctaagcataggcaaaaacaaggtctttgtgtcacccataagataacagacaccccctttctggataaaatgtacaacagctactttttttattattatactctaagttctaaggtacatgtgcacaacgtgcaggtttgttacataggtatacatgtgccatgttggtttgctgcacccatcaacttgtcacttacattaggtatttctcctaacgctatccctcccccagcccccccacctctcaacaggccccagtgtgtaatgttcccctccctgtgtccatgtgttctcatttttcaactcccacttacgagtgagaacacgtggtgtttggttttctgcccttgtgctattttgctgagagtgatggtttccagattcatccatgtccctgcaaaggacgtgaactcctccttttttatggctgcatagtattccatggtgtatatgtgccacattttctttatccagtctattattgatggacatttgggttggttccaagtctttgctattgtgaatagtgcctcagtaaacatacgtgtgcatgtgtctttatagtagcatgatttataatcttttatatatacccagtaatgggattgctgggtcaaatggtatttctagttctagatccttcaggaatcttcacactgtcttccataatggttgaactaatttacactcccaccaaaagtgtaaaagtgttcctatttctccacatcctctccagcatctattgtttcttgacgttttaatgatcaccattctgactggtgtgagatggtatctcactgtggttttgatttgcatttctttgatgaccagtgatgacaagcattttttcatatgtctgttggatgcataaatgtcttcttttgagaagtgtctgttcatatcctttgctcactttttgatggggttgtttttttcttgtaaatttgtttaagttctttgtagattctggatattagccctttgtcagatggatagattgcaaaaattttctcccattctgtaggttgcctgttcactctgatgatagtttcttttgctgtgcagaagctctttagtttaattagatcccatttgtcaattttggcttttgttgccattgcttttggtgctttagtcatgaagtttttgcccatgcctatgtcctaaatggtattgcctaagttttcttctagggtttttatggttttaggtcttacatttaagtctttaatccatcttgagttaatttttgtataaggtgtaaggaagggatccagtttcagctttctacatatggctagacagttttcccagcacatttattaaatagggaatcatttccccattgcttgtttttatcaggtttgtgaaagatcagatggttgtagatgtatagtgttatttctgaggcctctgttctgttccattggtctatatatctcttttggtaccagtgccatgctgttttggttactgtagccttgtagtatagtttgaagtcaggtagtgtaatgcctccagctttgatctttttgtttaggattgtcttggctatgcgggttcttttttggttccatatgaaccttaaagtagttttttccaattctgtgaagaaagtcattggtagcttgttggggatagcattgaatctataaattaccttgggcagtaggaccattttcgcaatattgatttttcctatccatgagcatggaatggtcttccatttgtttgtgtcctcttttatttcactgagcagtgttttgtagttctccttgaagaggtccttcacatcccttgtaagttggattcctaggtattttattctctttgtagcaattgtgaatgggagttcactcatgatttggttctctgtctgttaatggtgtataggaacgcttgtgatttttgcacattgattttgtatcctgagactttgctgaagttgcttatcagcttaaggagattttgggctgaggctatgcggttttctaaatagacaatcatgtcgtctgcaaacagagacaatttgacttcctctcttcctacttgaataccctatattgctttctcttgcctgattgccctggccagaacttccattactatattgaattggagtggtgagagagggcatccttgtcttgtgccggttttcaaagggaatgcttccagtttttgcccattcagtttgatattggctgtgggttcctcataaatagctcttattattttgagatacgttccaacaatacctagtttattgagagtttttagcatgaaaggctatcgaattttgtcaaaagccttttctgcatctattcagatgatcatgtggtttttgtctttggttctgtttatgtgatggattatgtttattgatttgcgtatattgaaccagccttacatcccagggatggagccgacttgatcgtggtggataagctttctgaggtgctgctggattcggtttgccagtattttattgaggatttttgcatcgatgttccttagggatattggcctaaaattatctttttttgttgtgtttctgccaggctttggtatcaggatgatgctggctcataaaatgagttggggaggattccctcttttccgattgattggaatagtttcagaaggaatggtaccagctcctccttgtacctccagtagaattcggctctgaatccgtctggtcctggactttttttggttggtaggttattaattattgcttcaattgcagagcctgttattggtctgttcagagattcaaattcttcctggtttagttttgggagggtgtatgtgtcgaggaatttatgcatttcttctagattttctagtttatttgcgtagaggtgtttatagtattctctgatggtagtttgtatttctgtggtatcggtggtgatatcccgtttatcattttttattgcgtctatttgattcttttctcttttcttctttattagtcttgctacaggtctatcaattttgttgatcttttcaaaaaaccagctgctggattcactgatttttttgaatggttttttgtgtctctatctccttcagttcttttctgatcttagttatttcttgccttctgctagcttttgaatgtgtttgctcttgcttctctagttcttttaattgtgatgttagggtgtcgattttagatctttcctgctttcttttgtgggcatttagtgctataaatttccctctacacactgctttaaatgtgccccagagattctggtacgttgtgtctttgttcttactggtttcaaataacatctttatttctgccttcatttcgatatttacccagtaattcaagagcaacagctacttttttttaccagtcatcacgtagggaagaaaaaatatcttctccttctacccttctaagttcttgactggggcctttgtaagaaaagagagattaacaatacaaaaaacatacaaatttattcaatataagtttacataacatgggaaccttcttaaggacatgaagaatggaagaaatgattaaacctgagcatttttatgctaggtttgatagagaatgaaaagtcataggaaaacctaataacacaaaggatatgagctgcaattccttcagcttaaaataattaatttttaaatatttaaattttaaaaatttaattaaagtgtcataatttggggtagcatgtcctgaactccatcaatagaattacccttgctttctgaaaacactcatactatgggggaaccaaacctccttctcctaaattctaaaactgtcttactgagacaccccatgatgtgtgttctctcttgctcagtgagcaatgaattcatcttgttcaaccacaaatgtccctgtgtctctggctgaagggcatatttcagagatcttaatagtctttattatgccttttggtgagcaccacttcttttttttttttcttttgagacagagtcttgctcttgttgcctaggctggagtgcagtggcatgatctcggctcaatgcaacttccgcctcccaggttcaagcgattctcctgcctcagcctcctgagtagctgggattacaggcacacaccaccacctctggctaatttttgtatttttaatagagatggggtttcaccatgttggccaggctggtctcaaactcttaacctcaagtgatccacctgcctcggcctcccaaagtgctgggattacaggcgtgagccaccccgcccagcccagtgagcaacacttcttaattttagttcgtttttaattttttgcctccctaatgagtgctttttatgttatatttaagaaatcctggcccggcgtggtggcttacacctgtaatcccggcactttgggaggccgaggtgggttgatcacgaggtcaggagatcgagaccatcctggctaacacggtgaaaccccgtctctactaaaaatgcaaaaaatcagccggatgcggtggcgggcacctgtagtcccagctactagggaggctgaggcaggagaatggcgtgaacccaggaggcggagcttgcagtgagccgagatagcgccactgcactccagcctgggcgaaagagcgagactccgcctcaaaaaaaaaaaaaaaaagaaagaaagaaatcctgccaggcacagtggctcacgcctgtaatcccagcactttgggaggccgaggtgggcggatcatgaggtcaggagatcgagaccatcttggccaatacggtgaaaccccgtccctactaaaaatacaaaaattagctgggcttcgtggtgcatgcccatagtgccagctactcaggaggctgaggcaggagaatcgcttgaaccccggaggtggaggttgcagtgagccgagatcgcgccactgcactccagcctggtgagagagtgagactccgtctgaaaaaacaacaacaaaaaaatcctttcttactccaatatcatacaggtatttttttttttaagtttccatttttttttttttttttttttgagactcagtttcactcttgttgctcaggctggagtgcaatggcacgctctcagctcatggcaacctctgcctcctgggttcaagcaattctcctgcctcagcctcccgagtagctgggattacagacatgtgccaccatgcctggctaattttgtatttttagtagaaacgagctttctctatgttcgtcaggctggtcttgaactcccaacctcaggtgatccgcccacctcggccttccaaagtggtgggattacaggcatgagccaccacgcccggccaacttgacaagtttttaaggtataattcattcagagtctaaaagtttagtaggatcatggtagtcaggagaataatttcactgcagaggtgtgtatgtgtttagcaaagtgattgctaacagattttcctgaaatccccgacagcacaaagtatcctttacattattatctttcatgcatataaaatctcacatatttctgtatagtgggagaacctggactttaacattttatatgaaagaaatgcataaaccctgtctttagaagataatgtaagatttggaaacaatatggtaaaagtaggaatagaacaaaagggggaaaatgacaaaagaaatttttttaaaaaagtccatgggattcgtgggtttctcagcttaaagggacaagggaaagcaagtctaggatgactcccagtattcaaagttaccagaagcttgagatgatgaactgctaaggtagtgagtgtgccctattttggggaaaagatggttttctcttcaagtctgttgctccttatctttcctcaatgaatttctaaggagcattgaaatttgtgtgaaagaaaggcttccaaaagtaaataagtttaaaatccagggttttgtattatactttcagatttccacaaaggcccagtttctctgttttttttttaaagttaaaaaaaagttgaaaatcaaaagccccaagtgggaagtaatttgtgtgtatctaaatattgtatacattcactgttttatcccagcaatttataaactgggtgtgcagaaagcagcctggagcaaaaacattgtaatgggagttaagagatcaggattttaatgatggtctgctactaatctatacgaaaatggtttgtgtataggtttcagattccttaaatatgtagaataagactacataatcttcaagcttctctgcagcacttaagttcagattctacttaagcacttacggaatgtgtcagt

SEQ ID NO: 7 - DNA, 50000 bp, Homo sapiens
  human DYXC1 chromosomal gene region, nucleotides 50001-100000
gtaattccaatttaccctattccagatttgtagcaaagcatttgtcagatgaatcatagggtggtttctcttcagttttcaaactttggagcaaaacctatataggtgcttttggcggccttacagttaataggatttgcaggatacaatagttttagacaagctcctggtatgtgttgtgaacaaacgttttctgaagaggaatttggagcaaagcaactatttcagggaacagtttgcaaactggggagttgaagccttaggtgtaaaacaaaggtgcattccagagaacaaagggagggtttgggttttacagcaaaagtttctgcccaggttcccagtcaggttcgtttatacaaatgaacgattgaaccgcacttagttctgattggttgatacagctgagccctgattggttgaggcaggtgagccctataagacccaacactcaacagaggtgtgggtttggggagaacgcagagtccatgtgtgacctctagtaggtaaatgaccacgtgtctctattttaaatttagacccagttagccgctctggatgttgacggattggctctttcaggttcacatttgttcacagtctgtatgaccaagaaatttgcaaaacagaacaactagcgctaactcttaatagcctcagtcagaataaacagacaagacactgcaccaagggcgtatgggcattgcacctgctcctcatgggctgctttcccgagccttatgcccacagccctccttagcattatcgcattatgaggctggaagtgcttttttttttctttctttctttttcttttctttttttttttttgagacggagtttcgctcttgtttcccaggctggagtgcaatggcgcgatttcggctcactgcgacccctgcctcccgggttcaagcgattctgctgcctcagcctcagcctccagagtagctgagattacaggcatgcaccaccacgcccggctaattttgtatttttagtagaggcggggtttctccatgttggtcaggctggtctcgaactcccaacctcaggtgatccgcccgcctcggcctcccaaagttctgggattacaggcgtgagctagcgcgcccgacccacagagtgcttttttttttttttttttttctcaagcagagggtatgggtctacttctttgaacattcgctcagatcgtaaggtaaccccaagaatcggcatcactctctggtctgggcgcctggatagtcacgcatggagtccgcgaggtgctggcgccggagcacgctgggaacgccgggctcccggccggcctgacccggaagggctggcgcatggtaaccccagcttccctagcaaccaagcaggcgcaagaagcaaccagccgcgctatccgctcccgttgctaccggaatgcctcttcaggttagcgattacagctggcagcagacgaagactgcggtctttctgtctctgcccctcaaaggcgtgtgcgtcagagacacggacgtgttctgcacggaaaactatctgaaggtaagaatgcaagtcttgcctgccggtctccgggggtgtggtccgaagcttctgcaagagcgcagctgggcacgagcaacaggctcatctttgccgataaacgtcctaaatttttaatcctattaaaaaaatgaatcccatacaggtaacacaccatctgtagacagataatcagatcagagactacttttatccagggcttcctcatatgccttgctaatctttcactacctggcataaatttagtgggccttttagaaagttattttacctttttcacgtgtttcttaatacgcgtgcttaatttgtgtaactaacttagtttctgttttcacccttctgcataggtcaactttcctccatttttatttgaggcatttctttatgctcccatagacgatgagagcagcaaagcaaagattgggaatgacaccattgtcttcaccttgtataaaaaagaagcggccatgtgggagaccctttctgtgacgggtggtaagttctttattaaaagatgttcatctggttgtaccttttcggtttcagaccttttaaaaaattttattttgaagcacctatattgtgtaggggaagatagattttttttttttaagagtcttgctctgtcgcccaggctggagtgcagtgacatgatctcggcttactgcaacctctgcccccggggttcaagggattctcctacctcagcctctcaagtagctgggattataggtgtccgccaccacgcccggctaaatttttttgtatttttggtagagcgttttgccatgttggccaggctggtctcacactcctgaccggtcattaagtgatccacccgccttgacctcccaaagtgctgggattacgggtgcgagccaccacacccggccagatagactttccttttactctccaaggttcaatagctgggtctataaaataaactaacaacagaaggattgacagaagaaaatgtatagaaatttatgaatttttaatattagtgcttgagggcatcacagggaaaaaagtgaataccccccaagcggtgagatttgagagtttagggactgtcttaataggggaagggggaggggatgtaggccgcttagaacaggaaatgatttttaggaaagatgaatggatccttagaagaatagataggaaatgtgatagcttttaacaaaatttgtcagagtgtggtgttgacttgtagtttccttttctgggagaatagtcaatattccctggttaatgaaactccaggggaagagatttatgacacttgggttcctttggaaaatctgtttttaggcagataagaggagttctggcaaaacctctccctgcatttgctgctttcaagtgcctttaactcaaaatgatcgctatatgaaagcagcatattttggggtggcatatcctgaactcattcaattgctgttttagaaaaaaaaaaaggcttatgagcctaatttaagtgatacagtgcttcagcagccatttaaataaacctaaaggctgaattagtcaaagttagagcacattaagcagaagatgtgggctcatgtcttaagggtaagccctcctgatatatagcctttattgagtacttaactttgcgttaggtgttaacatgcctgtaattccatgaaacaacctgtcacttaggtattattaccctttttttacaggggaggctactgacatatggaaagtttgagtaattggcaaaatcacacagctagtgactggcagcctggaactggagccttagcagtttcactctagagcagatgttcccaaccctggctacatatttgaatcagctatggaattttaaaataatactctaatccagtgaaatcagtttctgggaataggactcaggtgttggaagttttgaaaagctttccaggaattataatgtgcagccaaagttgaaatccccagttgttgtttgtttgtttggaaagacagagtctcgctcagtcacccaggctggagtgcagtgatgcgatctcggctcactgcaacctccgcctccctaggtttaagtgattcttcagcctcagcctcccaagtagctgggactacaggcacgtgccaccacgtccagctaatttttgtattttttagtagagacaaggtttcactatatgttggccaggctggtctcaaactcctgacctcaagtgatccacccacctcggcctcccgaagtgccgggattacagccatgagccaccgcgaccggcctgacatccccagttttaaagctagttctctgtagtcttattaagctagttgtcagtgctttgtactaataccaagatcagcttccatagtcagtaattgtcctctactgctttagccctgttttcctccttccttctttagttgtcatgttttctaatactttatgtttactgcctggaatttattttttttaattttgagttttttaagagatagaatctcaccctatcaccaacgctggagtgcggttggtgttaccatagctcattgtaacctcaaactcctaggttccagcaattctcctgcctcgacctcctgagtagcagggactacaggtgtgcaccaaaacacctaggtaattgtttttatttttgtagagacagggtctcactatgttgccgataggtcttgaactcctaggctcaagtgatcctcccactttggcctcccaaatgctgggatttcaggcgtgagccactgcacccagactactgcctggaatttcttttaagcctttggcattaagccttgaatatggactcttgagggtagaaagagttgcaaccattctttctatcaaggctttcttggtatcttgtagtggaatttaatttttaatttccaagaccaaggtagaatttatttttctaagatcgggtattcagctcagagaatactggttaaggtaggttaacatagccagtttacttaattttttttttttttttttttttttttttagtgagccccagactacctaaagagttttaaagaatcaggttcctgttttttgacctcttacttcctctgtgactcagtaagtctatgttggggcctgaggatgtcataacacacccagtctgttttgacttcttacctaaaattgaatatacatttattttgactcttgtaaattaactttttctgtaaaggaccagatagtattttagacttcacaggccatatggtctctgtcatatctattcagctctgccattgtagcacaaaagtagccacaggtgattcatatttatggacaatgaaatttgaatttcatctaatttttaaatgccacaaaatgttattttgatttttttcatccatttaaaaatgtgaaaattattcaaggcttgtgggctgaggggaaacaggtggcaggtcggatttggcccatgggtcatagtttgccaacctctgatttagacaaatcaaaacagtcttaaaactgacttaagctcagcgcagtggctcatgcttgtaatcccatcactttgggaggccgaggcaggtgaatcacctgaggtcaggagtttgagaccagcctggccaacatggtgaaaccctgtctctactaaaaatacaaaacttcgtcaggcgtggtagtggatgcctgtagtcccagctacttgggaggctgatgcaggagaatcgcttgaacccgggtggtggaggttgcagcgagctgagatctcaccactgcactccagcctgcgcaacagagcgagactccatctcaaaaaaaataaaaaataaaaaaccactaatttatatcacaatgaacattgattctacttcagaaaccaggtttgagattcctttagaaatcaaactcagtttgccgggcgcgctggctcacgcctgtaatcccagcactttggtaggccgaggcgggctgatcatctgaggttgggagtccgagaccagcctaaccaacatggagaaaccccgtctctactaaaaatacaaaattagccgggcatggtggcgcatacctgcaatcccagctactcaggaggctgaggcaggagaatcactttaacccgggaggcggaggttgcggtgagccgagattgtgccattgcacttcagcctgggcaacaagagcgaaactcagtctcaaaaaaaaaaaaaaacaagaaagaaatcaaactcagttttgccaggttacagtggctcatgcctaaagtaatctcagcactttaggaggccaaggtgggaggatcatttgagcccaggaattcaagaccagcctgggcaacatagggagacttcacctctattaaaaaaaaaaaattagccgggcatagtggcatgtgccttatagtcccagctacttgggaggctgaggttggaggatcacttgagcccaagaggtggaggctgcagtaagccatgctcataatgctgcactccaacctgggtgacagtatgtgatcctctctcaaaaaaaagaaatcaaactcaattttacttgcaattcagtgggattatttatttaaccatttttttttcacataaaatattgtggaactccagtttatacacgtagactgtctacctcattattcagactctaatttttctcattttttctggaaattccttcttgatacactctttatttctattagacatggagtatttcatttgggaagttaatgttcttccagttatttctgccgctgaagctgacttagctgtctaccttcctgtttttaaacagcaggagagatgagaagaggcttcagacctaacttcagtactagttcttagccaagatcttagatgaataaaattacacacttttacttacctctaactgatatttaacattctcttcaattgtgaagataggcaacaaataatagtagtatcttagtctgtttgggtactataacaaaataccatcaactgagaagcttataaacaacagaactttatttctcacactcctgtaggctgggaagtccagatcaaggtcctggcaggtttggtatgcggtgagggctcactttcctgtagatggcaccgtactgtgtcctcacatggtggaaggggcaagtctgctttctggggccttctgtataaggacactacctcccaaaaggcctcacctacaaataatatcacattaagggttgggattttaacataagagttttagggagacataaacattcagaccatagcaagtagtatcaacaaaaacaaaagtatgtttatatcagagatacctcttttatactctactttgaaattacaatattagacttgttgctatatattaatgcattaataaagaagcacataatcgtgtatcacaatttggccggccgcagtggctcacgcctgtaatcccagcactttcggaggccaaggcaggcggatcacaaggtcaggagatcaagaccatcctggccaacatggtgaaaccccgtctgtactaaaaatataaaaattaggtgggcgtggtgatgcccacccgtagtcccggctacttgggaggctgaggcaggagaattgcttgaacccaggaggcgggggttgcagtgagctgagatcggaccacagcactccagcctggcaacagagcaagactccggctcaaaaaaaaaaagtatatcacaattttatttcttaatgttttataagtgttttgatataactgagtttctttgtagacctgtatattttatttcatacattttaaaacattgtgaaaggatttataaggattcactactctgccaaggtgtctgctgcacaaaaaaggttaagaacccatgcctggggtatgattcatccctctacccaccccttatctactctcccctactttgtcttcccctcaagttttctcctctccccttctgtttttttttttctgcttaaaggctagatcttttcccttaggctctgatccttccctttgtaagataatttacccattgatcgtagtcttcagtttgtgaaactttgggccacatcaaatataaagataactgtgagatcttgcgtggtggctcatgcctgtaattccagcactttgggaggccaaggtaggagcatcgcttgagcccaggagttcaagaccagcctgggcaatatagtgagacctcgtctctacataaaatttaaaaattagccagatgtgttggcacacacctgtagtcccagctactcaggaagctgaggtaggaggattgcttgagcccaggaggttaaggttacattaagctgtgattgcttaactgcactccagcagcctgggtgacagagagagacctagtctcaggaaaacaaacaaacaagctgtggatggtaaggttttatatatatttatatatattatataaaatattatatataaatatatataaaaccttacctattatattattatatactattataatataatattatatattatattatatactattatataataatagtatataatattattatataataatataatattatctataatatataatatatattttatattttatatattatataaatgatgatttattgtgatcaggtttataagcaaatagccctagtttcttaacagagaaggaagaggataaatgtgtccttgcaccttcatcctcaccctctccctctcattacctgcctcttgtgtccctcccccatcccttcttcttcctctccacccccttcattctctagtcattcattattattacagttagttgcttaccttttaacattagcttcttggccagtttaatattgaaaaatattttccatgttccttgtgaagttgggtacaaagaaatctcatcctgggtcagtttatttaagtagttttctcataagtaaagcaaatttgtcataattctaatttagcaatattctgtacattttagttgacaaagagatgatgcaaagaattagagaaaaatctattttacaagcacaagagagagcaaaagaagctacagaagcaaaagctgcagcaaagcgggaagatcaaaaatacgcactaagtgtcatgatgaaggtaagttgcagaatgacaggcaagtcctctaaagagataatagtagactatgtgaggatagtgagttccagctgctggacttcctcagcattttcttcatactttgtttagaggacttactatgttatatagactgtctgctatttatatgtgtgtttttttttttcttgagaccgagtctcactctgtcgcccaggctggagtgcagtgcggtgatctgggctcactgcaagctccacttcccgggttcatgccattctcctgcctcagcctcctgagtagctgggactacaggcacctgccaccatgccaggctaatttttttgtgtttttagtagagacagggtttcaccgtgttagccgggatggtctcgatctcctgacctcgtgatctgcccgtctcggcctcccaaagtgctgggattagaggcgtgagccaccgtgcccggcccatgtttgttcttgaaatctgaatgaaagtcaaaggtgtaaaagttggacagttgtttcagggagagggttctatttctaggggagtaaaaataaagtttcaatggtgcaagtgttttacttctttttaaatcttggaataacagatatgattttaaaacagaagcatatttaaaacattattgtcaaatttttttaactgttaagggttttaagtgaaagttttaatcagcaatatattttttgagaaacaattcacattccatataatttacccatttaaagtgtacaatttaatagtttttagtacattcagagttgtgtaaccattactacagtcaattttagaacattttcatcaccccccaaaaagcaccaatacccctaagcagccactccccatttcccctaacacttagtcctaggcaactgcttatctattttctgtctcaatagatatacctattttgagtaatttttacaaaaaaattcttatctcaggccaagcgcagtggctcatgcctgtaatcccagcacttttgggaggccgaggcgggcggatcatgaggtcagtagttcaacaccagcctggccaacacagtgaaaccctgtctctactaaaaatacaaaaatcagccgcgcgtggtggtgggcacctgtaatcccagctactcggtaggctgaggcaggaaaatcgcttgaacccaggaggtggaggttgcagtgagcggagattgcgccactgcactccagcctgggcgacagagtgagacgccgtctcaaaaaaaaaaaaaaaacttcttatctcgaattcccattttgacatgttgcagtgactaaggaatctataatacgcattatttaagatttttgtggacccatgactgttttttggattttcataactagcattgttttctattatcaacagtttatgattccaaagatttgtccactttttttatcacttccacttaactttttttttttttttttttgaaatggtgtctggctctgttgcccaggctggagtgcagtggtgagatctcagctcactgcaacctctgccactcgggttcaagagattctcatgcctcagtctcctgagtagctgggactacagatgtgcaccaccacgcctggctaattttttatatttttagtagagacagggcttcaccatattggccaggttggtgtcgagctcctgacctcaagtgattcacccgccttggcctcccaaagtgttgggattacaggcatgagtcaccgcactgagccatatactttgtcttttacttaataattgaactggtcagctggtcaaagattcctgcctttagcatacttaattccatcttttctttattgtatgatcacagctgttggccttttgattttatcattttcatgaatttaaaaaaattaatatagaatttgtggcaatagatattcactgaaataataaaacagttaaataatggcacacttactgctgtattggtatgtcaaaacagaacagttgaccagcaggaagcaccactatgttgtaccttgaaggtttgttttttgttttttttttttgagatggagtcttgcttagtcgcccaggctggagtgcagcggcgcaatctcagctcactgcaagctctgcatcctgggttcacaccattcttctgcctcagcctcccgaataggtgggactacaggtgcctgccactacgcctggctaatttttttgtatttttagtagagacagggtttcactgtgttagccaggatggtctcgctctcctaacctcgtgatctgcccgtcttggcttcccgaagtgctgggattacaggtgtgagctgccgcacccagctgaagttttcaatttactaatgtatgaaagcttgactttggtattgggaaaggtgatttagaggaattaatatataaaatatgaagatacgggttatattaagaggacagaacctacaaaagccagtatcctgttccacaaattgtctgttttctctcttccctcagtagtgttgagagttgggagtatgttttgtttcatccttattatagccattatcaagtaaattggtgctcaaacatttgaataaattaatatgaatgcattactctagtactatgtacccgtagcaaacgtctactttggtgaattagctgaagctattttcactctgtgcaatcctagacactgttaagaaaagaactttgacgatccagtaagagcaaatagtaacaaaaggcaacatattgttagcttaagttagcataaattaatttaaaacttgaaggacatctaccaaaaataagtggtttttaagtagtaaccttttaaaaaataattagtggggctgtgtcaattcgctatactgtgtgtacaggtattattctgcttgggctaccctaacaaaatgttgtttaacagaaatttattttctcatagatcaaggtctggtagggtctctttctggttagactctctgtttttggggccttcttgctgcgctcttacgcagcaaaaaaaaaaaaagaaaaagaaaaagattggtatttcatcctgagagtatggtcaaggaatccaaagcagtattgtttgtgctgaatcctggaaaatgaatcacaattaggttcctggtagaaagagagggaaaacctcattgcagaagtgcttttaaagcccctgctgtgtcatatttgctaatgtaccattagccaaatcaagtcacatagccaagtccagatttaaagattggaaaaatagactatacttcttggaaagagaggtataatgggaaaatgacattacaatttggataggtatatgaaaataaggcacacaaaggaacatttattggtcagaaataatatcctaatgtgtccagaatttattccctctggtgggttcttggtctcactgacttcaagaatgaagccgcatacctcatggtgtgttacagctcttaatggtggcgcgtccggagttgtttgttcctctcggtaggttcgtggtcttgctgagttcaggaattaagctgcagaccctcacggtgactgttacagctcttaaaggtggcgcggacccgaagagtgagcagcagcaagatttattatgaagagtgaaagaacaaagcttccacagcatggaaggggacccgagccagttgccgctgctggctggggtggccagcttttattcccttatttgtccccgtctatgtcctgcggattggtccattttacagagtgctgattggtccattttacagagtgctgattggtgcgtttacaattgttaagctaaacacagagcactgattggtgcatttttccagaatgctgattggcgcgtttacaatcctttagctagacacagagcactgattggtgcatttttgcagagtgctgattggtatgtttacaatcctttagctagacacagagcgctgattggtgcatttttacagagtgctgattggtgcgtttacaatcctttagctagacaggaaaagttctccaagtccccacttgacccaggaagtccagctggcttcacctctcactaagattttgaaaaatttctcaaaggttatagagaactctcttaagggattttccacaaaagtgtaaaattagttgagtttgtcttttagaaagctcgttctggctaatgagtttgaagaggttgcagggaggaggcagagtttttgttaagatggagaaattttgagcatacgtatgttgaggaaggaagccagtaaagagggaaaggttaataatacaatgaagagagaagattgatgatggagcagttttctggtgatatggggatattttatcatttttgctaattaaaaataaacttttttttttttttgagacagggtctgttgcccaggctggaatgcaaaatggcgcaattacagctcactacattgacctcgcaggttcgagagatcctcccacctcagcttcccaagtagctaggactataggtgtatgccaccatgccctgctaatttttttgtagtttttgtagagatggggtctaactatgttgcccagcaggaacaaagaatagatatctaaggatctggaaaattcttaagcacacagataattaaaggaatttttactttgcttggtaaagaagcagggtcatctgggcatggtggctcatgcctgtaatcccagcactttggaaggccgaggcaggtggatcacctgaagtcaggagttcaagatcagcctggtcaatatggtgaaaacctgtctctactaaaaatacaaaaattagctgggcgtggtggcaggcacctgtaatcttagctactctggaggttgaggcaggagaatggctggaacccaggaagcggaggttgcagtgagctgagatcatgccattgcactcccgcctgggtaacaaagagtgaaacttcatctccaaaaaaaaaaaaaaccaaccaaccaaccaaccaaacaaacagaaaaacgaggcagggtcatcaacagaaagggagagattgaggtggaggtatagggatatgaggtgattagtggaagcttggaatactgatgccagagtcttccaaaatgtgcttttttttcctcttcgctaatcttggctgctactgcctccttttttttttttttttttttgagatggagtctcgctctgttgtccaggctggagtgcagtggcgcagtcttggctcactgcaagctctgcctcctgggttcatgccattctcctgcctaagtttcccgagtagctgcgactacaggtgcccgccaccacgcccatctaattttttgtattttttggtagagatggggtttcaccgtgttagccaggatggtctcgatctcctgacctcgtgatttgcctgcctcagcctcccaaagtgctgggattacaggcgtgagccactgcgcttggccgcctacttttcctgtcttcttcactattttgttctgtacattacccacttctatcttgaattttttttttatcatttcttgtttttgttttgttttccgcttcaatggaactcttttttccaactgatcttttaaactggataggtcaaatttgaagtgcatttttctgctgtggtttcttaaagttgaaacccagaaaccttaatctttgaaagaggtttgcctgatcatgcagataaggtatgagttgctagttgtctatttaatgccataggacctcaaaatgatccttatcagtgaatacacatgccctgtttatcatgggcatgtataactaatgtacaaaaagaccacaatgaactgctttgccattttattctattttatgataataactttcatgattctggcatttaaaaaatcctcccatctttcttttttctttcatctcattcctcttcagtgtatgtcagactttctcagcagcctgtcttccacttttacatgttttgtattttctccataaacccgttcttagagctctgtacttttggtatctcctgttctgatgtctttggccagtgtttattcatttatgtgttattggacacttgggttgcttccaccctttggctagtgtgaataatgctgctgtgaacatgatgtgatggttaatactgactgtcaactgattggattgaaggatgcaaagtattgatcctgggtgtgtctgcgagggtgttgccaaaggaaattaacatttgagtcagtgggctgggaaaggcagacccacccttaatctgggtggtcaccattgaattggctgccagcatggctagtatataaagcaggcagaaaaatatgaaaagacgagactggcctagcctcccagcccacatcttttgctcgtgctgggtgtttcctgccctcgaacatgggacttcaagttcttcagttttggaacttggactggttctccttgcttcacagcttgcagcaggcctattgtgataccttgtgattgtgtgagttaatacttaataaactcccctttatatatatctatctatactattagttatgtctctctaaagaaccctgactaatatacacgagtataaaaatatccctttgaaatcttgctttcaattgttttgggcttgtatctagaagtggatatagcagatcatatggtaatggtaattctgtttttttttttttttttcctgagacggagtctcactctgttgcccaggctggagtgcagtggtgcgatctcggctcactgcaagctccgcctcccaggttcaagccattctcctgcctcagcctccccagtagctgggactacaggtgctcaccaccacgcctggctaattttttttgtatttttagtagagacagggtttcaccatgttagccaggatgatctcgatctcctgacctcgtgattcacctgtctcagcctcccaaagtgctgggattacaggcatgagccaccgcgcccggcctatttttaattttttgaggaattgccatactgtattccatggcatttggaccattttatgttttcatcacagtgtgcaaggattccggtttctcccattctcactaacacttgttattttggttttttgagagtagccatcctaatgggtgtgaagtggtatctcattgtggttttgatgtgctcttcagtgccattttaatttgaactatatctctgaaacccactcattcccttttttcctaaatctttgagatattggtaagtataacatttattcttgcttgctcttttcctctcttctcccattatttggcctatgctttctcttaaaaaaaaaaaaaaaaaaaaaaaaaaaaaaaaaaaaaagctttattggctgggtgtgatggctcatgcctgtaatcccagcactttgggaggctgagcaggtggactgcttgaggtcaggaatccgagaccagcctggcccatctctactaaaaatacaaaaaattagccgtgtgtggtggtgtgcacttgtaatcccagctactccggaggctgaggcaggagaatcacttgaacccagaagacagaggttgcagtgggccaagattgtgccactgtattccagcctgggcgacagagtgagactccatttcaaaaacaaaaacaaaccccagctttattgagatataatttatataccataaagttcattcttttaaagtgtagaattcattgtttcttagtatgtatacaagttattcagccatcaccactatgtaattctggaacatttcataaccataaaaagagacctattggccaggcatggtggctcacacctgtaatcccagcactttgggaggctgaggggggcggatcacaaggtcaagagtttgagaccagcctggccaatatggtgaattcccatcactattaaaaatacaaaaattagccaggtgtggtggtgtgcatctgtagtcccagctactcggaaggctgaggcaggagaattgcttgaacctgggaggcagaggttgcattgagccgagattgagccactgcactacagcctaagcgacagagcaaaactctgtctcaaaataaataaataaataaataaataaagagccctattttcattagcagtcactccctacttttccctccaccatagcccctggaggagacagaatctactgtctgtttctatagatttgtttactctagacatttcacatacagtgtgtggtctttggatgtgatgcccatcagtctttatgggaggctttctttcacttagcaaaatgtatatagggcttatacatattgtagcatgtataagtttttcattcctttttatggatgaataatatggcatagtatggatggcaccacattttgtttatccattcatcagttggtgaaaatttaggttctcttcaccttttggctattacaaatagtactactatgaccattcatgtacttctagtatttaagtatctgttttttgttttgttttgttttgtttttgagatggaatcttgctctgttgcccaggctggagtgcaatggcatgatctcagctcactgcaacctctccttcctgggttcaagtgattctcctgcctcagcctcctgagtagctgggattacaggcaagcaccaccatgcccagctcgttttttgtattttttggtagagagggggttttgccatgttagccaggctggtctctaactcttgacctcaggtgatccacccacctcggcctcccaaagtgctgagattgcatgcatgagccaccacgcctggccttgagtgtctgtttttaattctttcatgtatatacttaggaatagaattgctgggtcatttgtaattctgtttagctttttggggaatcatcaaatagttttccacaatggctatactatttttcattcccaccaacaatgtatgatgattctaatttttttacatctttgccaacacttctgttgttgccagatggaaagtcttgactatgagttgtcccagttcttggcacattaaacaaagaattgaacaaaatgcacaaacaaagcaataaaagaacaaaacaacgaaagtgcaggtttactgaagcaaaagtacactccacagagtgtaagcgggctcaagcaagcagctcaagagccccgattgcaatgttctttcaggtttgtactaaactaaaagaatttggtagcattcctgggtaccgtttagaggcttcccattggttgcaccctacacaaatgaaggcttggcccattactaatcagaggctgaagtgaagtcttggcccataactaattagcaataggggagagagggggttttgtagagggaggggcctctggcccttggcatggagaggtggggtttttcttttggcccaattccaataagccagctgctagttggcctcaggctccctgttccagaccctgttctgcctcatttccccctgagagacttgatcccaataaatctttatgggaggcagaggaactgatggtctgtcttctgtaattgcttcgtgcggattagggcagagtccctacatattggggaccacagaactcttgccctgctctgtctagtggagagagggtgacttcttgaaggctagggctggcatcgtcacctggaactggctagaagccctgctgcatgatcatttgaagcttaatgttctctaggtgagaagaaataaatctggttacatgattaaacaaaaatggtccaaaaatcaatgtaagtacaagtattaataatgtgctggctaggggaaggagccatgaaacccagcttagtatccttgaccaggggccccatgacttggacaactgttgtcatattttagaggcctggtcagctaattttggggcagcatcccttactaatcctgattggttgatatgaaaacagtgctttccctctaggaaaagatataagccacttttttcagcagttaggagatccagtccctgtattagtccattctcacactgctaatgaagacatacccaagatcgggtaatttataaaggaaagagttttcattgagtcacagttccacatggctaaggaggcctcaggaaactaccaatcatggcagaagacacctcttaacagggtggcaggagagagaatgagcaccagcaggagaaatgccataaaaccttataaaaccattagatcttgtgagactcactcattatcatgagaacatcatgggggaaactgcccccatgattcatttacctccacctggtcccacccttgacacgtggggattattacaatccaaggtgagatttgggtggggatacagagcccaaccttatcagtcctcttctgttttgtaaagtgactgccaaagagtctacctgattttgtaaggcatactttgggcagtgtcttccaagccgtctgaaagctccttggacaagcgttggtaataggatagagaagttggaagcccactatttcctgtttctacgcctgcagttattcgtaaccctacctgaaagggtatgagttgggtggcttgtttgtgcctgatggttgcagttaaaggtataatgagagactggttattgggagctatattgatctcgggggctaagtaaacaagtgtgcagtgcaaacaattggttccagtccatttggctagtaaacgtaagtaggaactagtcccacacaggaaaaaggctgcttgtttttcaagacaaaaattgttctctatgatgaacatgtaggttagcttgttgttttccttttcccaagtggttaaggtccctgctacagtggccctggttaaaggctggagggggccttgggtatcgggggatcccacccccagtatttcaacgtaggttctttctgttttccataagtgttggccagctgagaaataaagagagacagtataaagagaggaattttacagctgggccatcgggggtgacatcacatatcggtaggactgtgatgcctgcttgagcctcaaaccagcaagtttttattaagggtttcaaaaagggggtgtaagaacagggagtagatacaaagatcatatgcttcaaagggcaacaagcagaactactaataagggtgtaacaaagatcacatacttctgagggaacaggacaaagggcaaaagcagaactactgacaagggtccaacaaagatcacaaggcaaagggcaaaagcagaactactgataagggtctatgttcagcagtgcacatattatcttgataaacatcttaaacaacagaacacaggattcaagagtagagaactggtctgaccacaaatttacgagggtggagtttttcccccaccctagtaagcctgagggttctgcaggagaccagggcgtatctcagtccttatctcaactgcacaagacagacattcccagagcccagagcggccatttatagacctccgcctaggaacacattcctttcccagggtattaatattaatattccttgctaggaaaagaatttagcgatatgtctcctacttgcatgtccatttataggctctctgcaagaagaaaaatgtgactctttttgcctgaccccacagacagtcagaccttatggttgtcttcccttgttccttaaaaattgctattattccgttctttttcaaggtgcactgatttcatattgttgaaacacacatgttttacaatcagtttgtacagttaacacaattatcacagtggtcctgaggtgacatacatcctcagcttacgaagataacaggattaagagattaaagtaaagacaggcataaattataaaagtattatttgggaactgataaatgtccatattaaaatgaaatctcccaatttatgttcctctgctgcggctccagccagtccctccattcggggtccctgacttcccgcaacacttgggaagtactctgagttgcccaagtggttttgttctctcagtttaggtggttatgcttagtgtctaccagccaacacccagagctatttttgtactgggggactaaaaggcaactagatgtctcaggattagtatagccttcccaggggaaaatgtgcatacagctagtgggcctaccctgacagtacttagactgcatatcctttaaagtagccttaactaagggtggttttcatgaaaaccatacaagttttctaaatgatgcagtctgggtaactgcatagcttacatgatcagataagaggttaatctctagggggtggttgcattgaagatatttcaaggtactcagggcttgaccaaaattttggcttcttttaatacaaagtggtgcctggaattttagttctgtgtgcatcgatattgggcccctaatgggtttttgaggggatgcaactccagaaaggttgatttgataggactggagaaggtttactgcttgtcttgtcattgtaacctttgtcatttccgtaattttgaagtcaaatatgtcatgcaggttcattaaattgaagggagttcttctcttatagttggagatttgggtggggatattttctcaagggcccaagattgggctgggattgggatagcagtgtgtttgacttcagcttaattggatgttgtttttggttgggataatgactgggtcctttaagctgtatgttgactggcactgccattctagcccttccaggcctcccagtataccatgctagcaggttgacctgtttgttgacatggtccagaacgttgtctgcatcactgaccattagcacccaggcagtgcttaaattgtagcagtgcccctattttaaccataaaatctcttctgaacaaggggactgggcagcttggtactattagaaacccctgctggaaaatttgtttttcaaattggcaggttaaaggaggggtgaaatgcctgactttattttttccttcatttcctataacagtcattgtcctagtggagggctttcctgcataagcagtaagtacagagtaacttgcccctgaatcaaaaagaaactgaatttgtgtacccgttgtgtccagagttactcagggatcctcagtagtaatgatgatgttcctggacaggggcagtgaggaaggccctgggccccttcagtcttcatctagctcctgcttttgcactgctagagtcttaactgactgagctcctccgtgggagctggggcagtcagtcctccagtgctggggttacaggcacctgccatgatgcccaggtaattttttaattttttttttttttcagtagagacaggttttcaccatgttggccagatggatctcaaattcctgaccttaaatgatccacctgcctcagcctcctgaagtgctgggatttataggtgtgagctgccacacccagctaggagtgacagcttctaagctctttacatgtcaaagcctaaactgatgttcctatgttaattttttacctatatagataaaaaacatgcacatatatgtgtatatatgtatatatatatatacacacacacacgcatacatacatacatacttacatatatttttaaatatttttaaaatttatttttcagtggttgttctgtgttcctttaactgatcacaatctacttcaggttaatactgactcagttccagtaaaatatagcaacttgttctaatataactccattttctactcctccctttggtctattagtgccattcaggtatgtgtatatgcatatatatttgttgtaatcccaacaatatgtgttatgattttacattaaaaatcattatttcttaaaaaatcttaggtttttaaataaataaagagtagaaagcaaaaatatatatattgtctttttaaaaagtttattttctaattgtggtaaaatataactaacatacaacttaccatctttaagtatacagttccatggcattaagtatattgacaaccatcactactaaccatctccagaactcttttctttttttattttttttttgagttggagtttcattctgttgcccaggctggagtacagtggcgtgatctcagctcactgcaacctctgcctcctgggttcaagtgattctcctgcctcagcctcctgagtagctgggattacaggcgtatgccaccacgcccggctaattttttgtatttttagtagagacggggtttcaccatgttggccaggctggtctttaactcctgacctcaggtgatccacccgccttggcctcccaaagttcggggattacaggcgtgagccaccatgcctggccctccagaactcatttcgtcttgcacaattaaaactctgtacccattaaacaataactctctattctctatcctcccagcccttgacagccaccattctactttttctatgaatttgactattttatgtaaaaaaaaatgacttattttacttcatgtaagtggaattatacaatatttgtccttttgtgactgttttatattttttagcatactttgtcttcaaggttcatccttgttttagcttgtgtcagaatttcctttctttttaaggctgaatcatgttctattaaaacgtatagaacacatttaaaaaaatctgtttgtattgtcttttacgcttacccacatatttaccatttctcatactcttcattctttcttgtaggtatgaattagttaatatctggtatcattttcctttcctttctcttttaagagataaggtctcactctattgtacaggttggagtgcagtggtacattcatagctcactgtaacttcaaactcctggtctcaagcagtcttcctgccccagcctcctgagtagctgggactacacgcacatgccaccatgcctatctagtaaagatggggatactctgtgttactcaggatggtcttgaactcctggcttcaagcgatcctcctggcttggcatcccaaatcacacctgggattgcaggtgcgagccacctccccatgccctgcccttttttaaaaaagtgtatcttatagagcaggtctattagcaacatactccctcagttttcatttgtcttggaaggtattttgctttcacttgtgaagaatagttttgctaaatatagaatttgtggtttatagttctgtttttttctgtcggtactttgaacatgtcatttcattgtcttctatccattgtttcatgagagccttaaaaggtgatagggtcaccatgcatccgtagctctctcatttacataatctttccattaaatttctgactgcttaagcacttatcctaaccaggatctcaacctcagaccaactgcaaagttttcctctttattcccaaccaagcctagcacttttatccaccaaagctacaggttttcactgccctacccggaaatgagcccaccctttttagcaacaaactgctaggttctttttggtagtttcatgttggtatgggttgagcatttatgatcaaaaaatacaaaatctgaaatgctccaaaatctgtaacttttgagcaccaacacaatgctcaaaggaagtgttcactggagcattttggatttcagatttttggagtaaggatgctcagccagtgtgtattctgcaagtattccaaaatctgaaaaaaatccaaaatctgaaacacttctagttccaagcattttggacaagggatattcagccagtaaaattgccaggctcatcatcctagctgggagggggttttaggagcaacctcaggccaaagggcacagcttcttacctttcatacttagaactttagcagcttttctagactaaagacttttctctttgtagtcttcttttggttaatttccagtcccctgaaatggttgtttttgataattttatccagttttatccttaatatttgtggaggggaattgccaacctcttcatgttgccacaaaatctgcatgttgtctttgcagatgaattttatatttacttatcaagtttcctaagaagtcctcttggtattttgattgaatttacattgactttgtaaatttgcagcgcatcaacatacttataatattgagatttccctccaggaacatggcatttatctccatttatttggatatactttacatctctcagcaaaattatttaacacacatatatgacttgcttatatattttttgtttgtttgttttgtgtttgagacgaagtctgtctctgtatcccaggctggcgtgcagtggcgcgatcttggctcactgcaacctccacttcccaggttcaagtgattctccttcctcagcctccagagtagctgggattacggcgtgcgccactgcacctggctaactttttgtatttttagtagagacgaggtttcaccatgttggccaggctggtcttgaactcccaacctcaggtaatccgcccacctcaacctcccaaagtgctaggattacaggcatgagccaccgcccctggccttatgacttgcttatattttgctaagcttattcatttatgtttttctggtgattgctgatgaaattaacgtttccattgtagtttctaattatttattgccagtttgtaggaaagtaagtttgtttttataagtttgtataagttgatctttttttttcttttgagatggagcctcacgctgtcgcccaggctggagtgcagtggcataatcttggctcactgcaacctccgtcttctgggttcacgcgattcttctgcctcagcctcctgagtagctgggactacaggcatgcaccaccacgcctggctaatttttatatttttagtagagacaaggtttcaacatgttggccaggctgatcttgaactccttacctcatgatctgcccacctcggcctcccaaagtgttgagattataggcgtgagccatcgcgcccagctagttgattttatatgtgactgccttgctgaacttacttttggtttgtcagtttattattttggttattttaagtagtgtactattatttgcaaatgatgcaattctatctccttgtaatctatatttgtatttcttatttttaaaagtttatcatttaggttctgacctctaggaaaatagtaaaacattaggtatacttgccttagtgattttgctggaaatatttctaatatttcttttattttttatttttatttttttgagacggagtcttgctctgtgacccaggctggagtgtagtggcgtgatctaggctcactgcaacctccacctcccaggttcaagagattctcctgcctcagcttcccgagtagctgggactacagttgtgtgccaccacgcccagctaatttttttattttttgtagagataggggttttgccatgttggccaggctggtctcgaactcctgacctcaggtgatccacctccctcggcctcccaaagtgctgggattacaggtgtgagccactgcgcccggctatttctaatatttcgtcatgaagtataacatagtcttggtccttctatccagcttataaatttttatttcttatttttattcccaatatcaactgacattttggcatacactgacatcataatgcttttctctattgatttcctaataccttaagtaagaaagaccctatttgatcaaggtgtattataattttaatattttcctaggttaagttacccctatttgattaaggtgtattataattttaatattttcctaggttaagttacccctatttgattaaggtgtattataattttaatatttttctaggttaagtttgcatatatatatatatacacacacacatatatatacacacatatatatacacacatatatatacacacatatatacacacatatatatacacatatatatacacatatatatacacatatatacacatatatatacacacacatatatatatatacacacacacatatatatatatatatatatatattttttttttttaggttttttttttgtttgttttgaggtggcctctcactctgttacccaggctggaatgcagtggcgcagtctcggctcactgcaacctctgcctcttgggtttaagcgattctcatgcctcagcctcccgaatagctgggattacaggtgtgcgctaccacacctagctaatttttgtatttttagtagagatgggttatcaccattttggccaggctggtctctaactcctgacttcaagtaatttgcctgactcggcttcccaaagtgttgggtttacaggcgtgagcctccatgccgggctgagtttgctaatgttttatttacaatttttgcatgtaattcacaagtgggattggcttataaagaacaatgccttgttgaccagacattcattctagattaaaagaattatacatttcctcttcttcaaaaatttagtgtaggaactattcattattctcgtattagttttatttgctgtctgcattaaactaataacctgtagtaaatggcatccttttttgaacagtacaaacacctaaaacccaatttgtttattttattttttttgagaagtctcactgtgtcacccaggctagagtgcagtggggcgatcttggctcactgtaacctctgcctccgagattcaagtgattctcctgcctcagcctcccaagtagctgggattacaggtgtatgctaccacacccacctaatttttgtattattagtatagatggggtttcaccatgttggccaggttgttcttgaactcctgacctcaagtgatcaacctgcctgggcctcccaaagtgctgggatttcaggcatgagccactgcccccgggcctccagtcctttatgtacgatgattattattttagccttctgagtttttctgttactgcatattcctccgtgtgtcttccctgtattagatgatttttctttgaaagaccttcaaggattgtttaatatttgtcggtgtctgtgttaaattgtgtgtttcctatattagatgatttttatttaaaatatctttaagagctgggcacggtggctcacgcctgtaatccctgcactttgggaggttgaggcgggtggatcatttgaagtcaggagtttgacaccagcctggccaacatgatgaaaccctgtctctactaaaaatacaaaaattagacgggcatggtggtacacacctgtaatcccagctactcaggaggctgaggcaggataattgcttgaacccgagaggcggaggttgcagtgagcgaagatcgtgctactgcactccagcctgggtgacacagcaagactccatctcaaaataagtaagtaaataaataacaaaatatctttaagaattgttggctgggcgtggtggctcacgcctgtaatcccagcactttgggaggccgaggcgggcagatcacgaggtcaggagatcgagaccatcctggctaacacagtgaaaccccatctctactaaaaatacaaaaaattagccgggcgtggtagtgggtgcctgtagtcccagctactcgggaggctgaggcaggagaatggcatgaacccgggaggtggagcttgcagtgacccgagatcgtgccgctgcactccagcctgggcgacagagtgagactccatctgagaaaaaaaaaaaaaaagtttaacatttgtccatctctttatgacatcataaaaaatatatatttggtccctgcctcttgttattagcatagagctcctaaaacccatgtaattttctgagtgatggggtgctaggttcatcttttgttaaattatttggattttgacccaggttcctgacacagagctcctaatcccttggaatttcctatgtaataggaaaatcttttgttctaatgaggcaactcttgatgggctcttgggtggtggctggccaccagaaagaccaagctatgactagttgtctggaacttgcagcacccctccccatcattttgataggggctggagactgacttaataatcaatcatgctgggtccactggcttacgcctgtaattccagcactttgggaggccgaggtgggaggatcgcttgagcccaggagctcaagaccagcctgggcaacatagatagtgagaccctgactctaaaaacaacaacaacaaaattagccattcgtggtgctgcgggcctgtggtcccaactacttgagaggctgaggtgagaggatcacatgagcctaggaagtggaagctacagtgagctaaggttgtgccactgcactctccagcctgagtgacagaacaagaccttgtctcaaaaaaaaaaaaaaaaaaaaaatccgccaggcatagtagctcatgtctgtaaatcccagcactttgggaggctgaggtgggtagatcacttgagcccaggagttcaagaccagcctgggcagcaagggaaaaccccatctttacaaaaaaataacataaaaaattagtcaggcttggtggcatgtgcctgtgtcccagctacctagaaggctgaactggaggatcgttggagcccaggaggttgaggctgcagtgagccatgattgcgccattgcactccagcctgggtgacagagcaagaccctgtctaaaaaaaaaaaaaaaacccagtcatgcctgctagataaaacctccataaaattcctgtaactatgaggtttggagaacttctgggttgctgaacacttggagggagaaggcatggaaactctgtgctccttccccggtatctttccctagacatctctttcatttgcctcttcctgatttgcatctttttattaaaaatgggtaaatatggctgggcgcggtggctcacgcctgtaatcccagcactttgggaggcctaggcgggtggatcacctgaggtcaggagttcaagatcagcctggccaacatggtgaaaccctgtctctactaaaaatacaaaaattagccaggcgtggtggtatgtgcctgtagacccagctactcaggaggctgaggcaggacaatcacttgaacctgggaggcggaggttgcagtgagccgagatcgtgccactgcactccagcctgggcaacggagtgagactccgtctcagaaaaaaaaaaaaaggggtaaatataagcgaggtatttatctgattcctgtgagccattgaagcaaatggtcaaacctgaggagggggttgtagaaacccccaacttatagctgattggttagaaatatgggagtcctggacttatgattggcatctgtgtggagactgtcttatggaattgagcccttaacctgtgggatctaatgctaactccgggtagataatgacagaactgaattagaggacacatagctgatgttagagaattggtaagcataggccgggcgcagtggctcacgcctgtaatcccagcactttgggaggtcaaggtgggcggatcacgaggtcaggagatcgagaccatcctggctaacacagtgaaaccccgtctctactaaaaatataaaaaaaaaataaattagccaggtgtggtggcagacacctgtagttccagctactcgggaggctgaggcaggagaatggcatgaacccaggaggcggagcttgcagtgagccgagattgcgccactgcactccagcctgggcgacagagtgagactccgtctcaaaaaaaaaaaaaaaaaaaaaaaaaagaattggtaagcataggagaaacccacaagtttcgtgatggaagtgacctatggagtgttgtgagagtatataagaggcataaacagtttgttttttctctgtacactctgtttttctttttctttctttctttcttttttttttgagaggagcctcgctcttgtcccccaggtttgagtgcaatggctttatctcggctcactgcaacctccacctcccaggttcaaatgattctcctccctctgcctcccaagtagctgggattaagggcctgccaccaagcccggctaaattttgtattttttagtagagacagggtttcaccatgttggccaggctggtctcgaactcctgacctcagatgatccacctgcctcagcctcccaaagtgctgggatttcaggcgtgagccaccgcgcccggtcactgtacactctgtttttctttggtacatgtagtctcttttctctttatcataagctttgtgttattttcttcttcatgtttttctcttcattgtagttccttctgttacctactctgtgaagcctttcttggtttctcctcttcatcactactctctgaagagctggactatttctgtctctaatatagcttattataaattatcattatacctatagttcccttaactacatcttggctataataccagaactttcctgttattttttgttgtaagaacagtctcatttttggtaattaagacttagtcttatgcattttaaaaacttcatgtaaaattcattcttcttgcagggagtatgatgggtgagggtgtctgactctccacatattctccctttttcatgtgttggggcagagagtgaaaaaaggcagacatataattaggctgggtgaagtcgtcttttaggttgtggcttcctctctaacatggagtgttcatcatgccttattgtgaggcatttaaattttgtttcattgccatgtgtagattcttcctggttcaatctgttggcagcttactggcaactctctgaactaagaatatatagtaataattagcatattttgtgcttaatttaagcctgaggtaaaattgggactacaggaaattttcattcagtgtcccgtgaaatttattttaaagtcagcatctcacaggaaaataatcagtcttctagaagagagtattatgtcatgtagatattacagagcacctttcagtgcacaggtttcatactcgtaatgttaaatctctattttaaagtaatacaacatttagggaccttcatcctagcatatccatagtctagaaccacttcagatgtcctatttaaatgtaaattaatttaaatgtacattaatgaaacttaaataacattaaaaattcagttcctgagtctcagtagccacatttgaagtacttaattagccacctgtggctagtggctactatattggatagcacagacattttgatcattgaagtaagtgcttctctagtggatagaggtaagaaatttttcttaccataggaaagtcagatacatggagtcaatcatgtacaccaactaggtactctgtttcacaaggttttttttcttgcccatgtgagggaagtttgaaaggcagaaaagttgccacacacctgtacaagatactgctttcaaattcatctgatatttagctctttgaaagtgtcagacaactactagtgcaaacataggttttgcagcaggattaagacttcaaatatttccacccagcagagacagaccgtcttgttcatccttcctatttccttttctactgttccctcttccttcagtccttaggactgaagggttggcacaacttcggttgacagggtaagacttgggataagttgaaaagcaaatgtttgaatatatgtgttgggatgtgtgtgtgtgtgtgtgtgtgtgtagattttgagagaagtggcttaactcctagaataataaatttctattattttttctaccaagcccttaggtatcatctaattgaatgcctgcccacattttacatataagtaaactgaggttggagaaagtgattgcctaagaccatacagctaatggtagatctgggactaaaatctttgttccatgactccaacaccaatggtttttctcctgaatcatttacctttcccattttaattcataattcactactacctattgttacaaatacaatggcaagagtttagaggtatgaataactaattattgtaatgaatttctcatttaaataaatgttgcttttatgtattttaaagaatttggtaattgcaattgttatagattgaagaagaagagaggaaaaaaatagaagatatgaaagaaaatgaacggataaaagccactaaagcattggaagcctggaaagaatatcaaagaaaagctgaggagcaaaaaaaaattcagagagaagagaaattatgtcaaaaagaaaagcaaattaaagaagaaagaaaaaaaataaaatataagagtcttactagaaatttggcatctagaaatcttgctccaaaaggtaaaatgattaagaatttcttgagtagtgagtcctgttggtgaagtatatatgacgcttttctttttttgtaaatctattcacagcattaggttactgttttggcacattgagaataagcaattcagtacaaagactaaagtaaagacatttcttctttcggggataatgaagttttctaataaaatgagcaaggattcgttcaaggattcgaaaagaagagtgtcttctttgcttgtgtgctttcttttgaaatttacaaagatgttgttaaataagtgcttaactcaggcccaaaaagacttggagtggactagcaatgtcagaagcattcatttattccattagagataagaaactaggaatttcattaaaagaccttttcctcacatcacttaggaaaatgagtactataagcagacttactctacagcaaataaagattgcttctagcttttaatcttggtgtattcaaaaacagataaattaatataatttttttttttgagacaaggtctggccctatcgcccaggctggagtgcaatggtgtgatctgagctcactgcagcctctgcctccccagctcaagccatcctgccttagcctcccaagtagctgggactacaggcatgtgcctcaatgtccagctaatgcttttttgtattttttgtagagacgaggttttgccatgttgcccaggctggtcttgaactcgtgagctcaagcaatctgcctgccttggcctcccaaagtgctgagattgtgtgcatgagccaccatgtccagctgatacaaatataattcagtttttgtatgtgtgtgtttttaaaattattatttcagtagttttggggaaacaggtggtgtttggttacatgtataagttctttagtggtgatttctgagattttggtggacccatcaaccaagcagtgtacactgcacccaatgtgtagtcttttatccctcacccctgcacccttcccccctagtccccaggcccatatcattcttatgccttggcatcctcatagctcagctcccatttatgagtgagaacatgggccaggagcagtggctcatgcctgtaatcctaacactttgggaggacaaggcgggcagatcatctgaggttgggagttcgagaccagtctgaccaatatggagaaaccccatctttactaaaattacaaaattaaccaggtgtggtggcacatatctgtaatcccagctactcaggaggccgaggcaggagaatcgcttgaacctgggaggcggaggttatggtgaaccgagatcgtgccattgcattccagcctgggcaacaagagcaaaactctgtctcaaaaaaaaaaaaaaaaaaagtgagaacatatggtatttggttttccatttctgagttacttcatgtagcataatcgtctccaactccatccaggttgccgagaatgccattattttattcctttttatggctgagtagcattccatgattgtatgtgtgtctgtgtgtgtgtgtgtgtgtgtgtgtgtgtatatatacaccatattttctttatcttctcattagttgataggcatttaggctagttctatatttttgcaattgcaattgtgctgctataaacatgagtgtgcaagtgtctaatgacttctttttctctagatagatacccagtagggggattgctggatcaaatggcggttctacttttcgttctttaaggcatctatctccacactgtcttccatagtagttgtactagtttacattcccaccagcagtgtaaaagtgtcccttttcaccacatctacaccaacatctgttattttttcatttttaaattatggtcattcttaacaggagtaaggtggtatcgcattgtggttttgatttgcatttccctaataattagcggtgttgagcattttttcatgtttgttggacatttgtttgtttgtttatttattttgagatggaatttcgctcttgttgcccaggctggagtgcaatggcacgatctcggctcaccacaacctctgcctcccaggttcaagcgattctcctgcttcagcctcccgagtagctaggattacaggcatgtaccaccatgcctggctaatttagagatggggtttctccatgttggtcaggctggtctcgaactcttgacctcaggtgatccgcctgcctcagcctcgaaaagtgatgggattacaggcatgagccaccacacctggcgggacatttgtatatcttcttttgagaattgtctattcatgtccttagcccactttttgatgggattgtttgttttgttcttgctgatttgtttgagttccctgtacattgtggatattagtccttcgtcagatgcatagtttgtgaagattttcttccattctgtgggttgtctgtttactgtgctgattatttcttttgctgtgcagaagctttttagtttaattaagtcccatctatttatctttgtttttgtttcatttgcttttggattcttggccatcaagtctttgcctaagccagtgttagaaggggttttccagtgttatcttctagaatttttatgatttcaggtcttagacttaagtctttgatccatcttgagttaatttttgtgttaggggagagatgaggatccagtttcatacccctacatgtggtttgccaattatcccagcaccatttgttgaataggttgtcctttccccactttatggttttgtttgctttgtcaaagattggttggctgtatttgactttatgtctgggttctctattctgttccattggcctatgtgcctgtttttataccagtactatactgttttggtaactgtggccttgtagtatagtttgaagtcaggtaatatgatcccctcagatttgttctttttgcttagtcttgctttggctatatgggctctttttttgttctatatgaattttaggattgttttttctagttctgtgaagaatgatgatgatattttgatgggaattgcactgaatttgtagattgcttttggcagtatggtcattttcaaaatactgattctacccatctgtgagcatgggatgtatttccatttgtttgtgtcatctatgatttctttcagcagtgttttgtagttttccttgtgagatctttcacctctttagttaggtatattcctaagtattttggttttttgttttgttttttgggtttttttgcagctgttgtaaaagaggttgagttcttgatgtgattctcagcctggtccttgttggtgtatagtagtgctactgatttgtgtgcattgattttgtattctgaaactttacttgaattcatatatcagatctaggagctttttggatgaatctttaggattttctaggtatacgatcatatcattggccaacaatgacaacttgacttcctccttactgatttagatgctctttctttctttctcttgtctgatcgctctggctaggacttccagtactctgttaaatagtgttggtgaaagtggacatccttgtcttgttccagttctcaggggaaacactttcaacttttcactgtttattataatattggccatgggtttgtcaaagatggcttttattaccttaaggtatgtcccttctgtgcccattttgctgagggttttagtcataaagggatgctggattttgtcaaatgctttgtgtgtgtgtgtgtgtgtgtgtgtgtgtgtgtgtgtgtgagacagagtctcactgtgttccttaggctggagtgcagtggcatgatcttggctcactgcaacctctgcctcccaaattcaagcagttctcctgcctcaacctccccagtatctgggattacaggtgtgcaccaccacacctagctaactttttgtattttttagtagagacagggtttcgccatgttggccaggctggcctcaaaactcctgacctcaaatgatccgtctgcctcggcttcccgaagtgctgggattacaggcatgagccaccacactcggcctcaaatgctttttctgcatttattgagatgattgtatgatttttgttttgacttctgtttatgtggtgtattatatttattgattttttgtgtgtgtatgttaaaccagccctgcatccctggtatgaaacccacttaattatggtgtattacctttttgatatgctgttgcatttggttagctagtatttggttgaggagtgttacatctgtgttcatcagggatattgttctgtagttttcttttttttttttttgttatgtcctttcctggttttggtattagggtagtactggcgttgtagaatgatttagggaggatttcctccttatcttttgaaatagttttaataggattggtaccagttctcttagaatgtctgttagaattcagctgttgggctgggcacatggctcatacctgtaatcccagcctttgagaggccaaggcaggcaatctcctgaggtcaggagttcaagaccagcctgcccaacgtggtgaaacctcatctaactaaaaatacaaaaattagcagggtgtggtggcatgtgcctgtaatcccatctactcgggaagctgaggcaggagaatcgcttaaacccggagatggaggttgcaatgagccgagatctcaccactgcattccagcctgggggcaagagaacaagaccctgtcttaaaaaaaaaaaaccagctgtgaatccatctggtcctggactttttttagttggccatttttaaattactgtttcaatctccctacctgctattggtcttttcagagtttctatttcttcctggcttaatctgggaggattgtttaattccttctaggttgaggaatttatccatctcctgtaggttttctagtttgtgtgcacaaaggtattcatagtagctttgaatgatcttttgtatccctgtggtatttgttgtaatttctctcatttcatttctaatcgagcttatttggatcttattcttttctgggttaatcttactaatggtctatcagttttgtttgtcttttcaaagaaccagcattttgtttaatttttcttttgtatttttttctttcagtttcatttagttctgctctgatctttgttatttcttttcttgtgctgggtttgggtttgatgtgttcttttttctctagtttcttgatgtgtgagcctagattgtctgtttatgctctttcagactttttgatgtaggcgtttagtgctatgaactttcctcttagcactgcttttgctgtatcccagaggttttgactggttgtgtcactattatcgttcagttcaaagaattttaaaatttccgtcttgatttcattgttgacccaaagatcattcaggggcaggttacgtaatttccatatatttgtatagttttgagtgttccttttgaagtcaatatccagttttattccactgtggtcttagagagtatttgatataattttgattttcttaaatttactgagatgggctgggcgtggtggctcacacctgtaatctcagtactttgggaggcctaggtgggtggatcacgaggtcaggagatcgagaccattctggtctctaaccagagaccagaatctggtggctaacacggtgaaaccccatctctactaaaagtacaaaaaattagccaggcgtggtggggggcacctgtagtcccagctactcgggaggctgaggcaggagaatggcatgaacccaggaggcggagtttgcagtgagccgagactgcaccactgcactccagcctgggcaacagtgcaagactctgtctcaaaaaaaaaaaattattgagatggctgggcgcagtggctcatgcctgtaatcctagcactttgggaggccagggaaggtggatcacctgaggtcaggagtttgagaccagcctggccaacatggtgaaaccttgtctttactaaaaatacaaaaattaggctggtgtggtggcgggggcctgtaatcccagctgcttgggaggctgaggcagaagaatcacttgagcccaagaggtggaggttgcggtgagtcgagatcacgccactgcactccagcctgggtgacaaagcgaggctctgtctcaaaaaaaaaaaaaaattattgagacttgttttgtgttctatcatttggtttatcttggaatgaatgttccatgtgctgatgaaaagaatgcatattctgcagttgttgggtagaatattctgtaaatatctattaagtccatttgttctaggatatagtttaaatccattatttctttgttgactttctgtcttgattacctgtctagtgctgtcagtagagtactgaagtcctccactattattgtggtgctatctcatttcttaggtctaataataactgttttataaatttgagaattccggccgggcgcggtggctcatgcctgtaatctcagcactttgggaggccgaggcgggtggatcacgggtggtcaggagtttgagaccagcctgaccaacatggtgaaaccccgtctctactaaaaacacaaaaattagccgggcgtgctggcacgcacctgtaatcccagctactcaggaggctgaggcaggagaattgcatgaatctggaggtggaggttgcagtgagccgagactgcaccacggcactccagcctgggcgacagagtgagactctgtctcaaaaaaaaaaataaataaataagtaaataaataaataaataaatttgagagttctagtgttaggtgcataaatatttaggattgtgatattttcctgttggactaattcttttatcattatataatgtccttttttatcttttttaactgttgttgctttaaagtctgctttgttttgtataagaatagctattcctggctaggcgcagtggctcacgcctgtaatcccagcactttgggaggccgaggcgggcagattacgaggtcaggagtttgagaccagccaggccaacatggtgaaactctgtctctactaaaaatacaaaaattagccaggcgtggtggcgcgtgcctgtagtcccagctacttggaaagctaaggcaggagaatcgcttgaccccaggaggcagaggttgcagtgagccaagattgcaccactacactccagcctgggcaacagaaggagactctgtctcaaaaaaaaaaagaaagaaagaaagaaagaaaaaagaatagctactcctgctgactttggtgttcatttgcatggaatatctttttccaccctgtttatgagtccttatatgttaggcaagtctgttaaaagacttacttggttggttgatttttatccatttgacattctgtatcttttaagtggagcatttaggccatttacattcaatatcagtattgagatgtgaggtactgttctattcatcatgctagttgttgctgtaccttattttttttcattgtgttattgttttatagatcctatgagatttatgttttaaggaggttctattttggtatattttgaggttttgtttcaatatttagaactccttttagcatttattgtagtgctggcatggtagtgacaaattcagcatttgtttgtttgaaaaagactttatctcgcctctggttatgaagcttagtttcactggatacaaaactcttagcggataattattttgtttaaggaggctaaagataggacccccatcccttctggcttgcagggcttctgctaagaaatctgctgttaatctgataggttctcctttatagattacctggtgcttttgcctcacagctctttttttttttttttttgagacagagtcttgctctgtcacccaggctagagtgcagtagcgtgatctcggctcactgcaacctccaactcccgggttcaagcaattctcctgcctcagcctcctgagtagctgggattacaggcacctgccaccacgcctggctgatttttgtatttttagtagagatggtgtttcaccatcttggttggccaggctggtctcgaactcctgacctcgtgatccacccgccttggcctcccaaagtgctgagattacagatgtgggccactgtgcctgaccttgcctcacagctcttaagattctttctttcatctttactttagacaatgtgatgactatgtgcctaggcaatgatctttttgcaatgaattccccgggtgttctttgagcttcttgtatttggatgtctagatctctagcaagaccatggaagtttttcttgattattccctcaagtatgttttccaaacttttagatttcccttcttcctcaggagcaccaattattcttatgtttgcttgtttacataatctcagatttcttggaggctttgttcattttttaaaattctttttttctttgtctttgttggattgggttcgtttgaaagccttgtctttgatctctgaagttatttcttctacttgttcaaatctattgttgaaactttccagtgtattttgcatttctttaagtgtgtctttcatttccagaagttttgattattttttctttataatatctatttctctggagacttttccattcatatcctcctttttttaaaaatttctttaagttgattttcaccttcctctagtgccttcttgaggagcttaataatcaaccttctgaattatttatctggcaattcacagatttcttcttagtttggatccattgctggagagctagtatgatcttttgggggtgttacagaaccttgtattgtcatattactagaattacttttctggttccttctcatttgagtagactgtttcagtggtaagatctggaactcaagggctactgttcagattcttctgtcccatgggtgttcccttgatgtggtgccctccccctgccactagggatggggcttcctgagagccagactgcagtgattgttattgcccttctatttctagccacccagttgggccccaggctggtgctggggaatgtctgcaaagagccctgtgatgtgatccacaggtctcccagccgtggataccagcacctgttctggtggtggcaggtgagtgaagtggattctgttggagtcttttgttgtaattttgtttagtgcacttcaagtactggttatgtaagcagtgaagttgtcacgtggacagattcaggacctctggctagacagggtgttgcaggaagtggaattagctgttgttttctccttctttggagcagggttgttctgttatgagttgctatggtggcttgagttcgttgggctccagccaggaggtggcactttcaagagggcaccagctgcagtatagaagagagatataatcttgatgttggccgggacaagtactcgggtttctcaggtgatgggcagggccatagagcaggtagagaaaaccttgccccatcaggtgggggcaaggttagttgggtctgggctcaggctccccttgggtggggcttgctgtggccacagtgggggtggaggggattctcaggccaatggaattatgttcccaggggcattatggctgcctctgctgtgctatacaggtcaccagggaggtgggggaaagctggcagtgacaggcctcacctagctcccaggcagccaccaaggccagtctcactctggccatgccaccccaaccgcactgcgtttatatccaggcagttggtgagcagggctgagatcttgccccagactataagccttgccactgagaaagcaagagggctgttaggcctcaccagtccctgcctgcccacactattggctgtggcttctgcactcgcatctgtacttcccatttgccccctctgccaccaatttctgcttaggaaaattcctgctcagtcaaaattattacaaagttcagctacaagcttccttcaccctgtggtcctgccccaattccactggctgccttcccgtctccaaggacctctgtgagatcacgccaggaatggcttccttggactccagctggggatggaagtacctatagggctcttcctgctgcttcttctactttcatattttgctcagctacctaaatctatttcagctcagggtaaagttaaatccttcccctgtgatctggattttcaggttccccagtggggatgtatgttcagagactgaccttttcttccctcacactttgggaactcacggtttttcagctgtttcatggcatttgcagcagcgaaccacttctttcaaagggtctgtgaattctttcagttttcctacagtgtttcttgcggtgttcctgaagtgattcttggagtaaaagttcgcattgtgagtctccagatgctgttctgtccatccaggtggcagctgcatgttaatcctgtctccgatctgccattttccctcctatctcccaatgaatataattctatactgaaagctacgagtttggttttatctaattttagaatattaaactttgttttggatgcattttccgccggatgcagtggctcacgcctgtaatcccagaactttgagaggctgaggcgggcagatcacctgaagtcgggtgttcaagactagcctgaccaacatggagaaaccctgtctctactaaaaatacaaaactagccggacgtggtggtgggtgcctgtaatcccagctactcgggaggcactgaggcaggagaatcgcttgaacccagggaggtggaggttgcagtgaagcgagatcttgccattgcactccagcctgggcaacaagagtgaaactccatctcaaaaaaaaaaaaaagaaatatttagtattttccttacataagtcattattgaattcctataaatttaacattttagtgtaaaatcacatatataataataataattaacatttattaggtgactactatgtgccaggggattttattgcttgggtcactcagtcttcacaacacttcagtctaagtgctgctattcttattttagaggaaatagatgttcagaaaaaaaaaataactatgaataagtaaacttgtgggatttgaacccagatctggtctgcatctgaaacctgtgctatttccaccaggtcatatgtctttacaactttaacagtgaggcttggcatattgaaaagtcatttagagcctgggcatggtggctaacgcctataatcccagtactttgggaggccgaggtgggcagatcacctgaggttaggagttccagaccaccctgggcaacatggcgaaaccctgtctctactaaaaatgcaaaaattagccaggtgtggtggcatgcgcctgtagtcccagttactcaggaggctgaggcaggagaatcacttgaacccaggaggaagaggctgcagtgagccaagatcgcgccactgcattccagtgtgggtgacagagtgagaccctgtctcaaaacaacaaaaacaaaaaaaagtcatttagattgaatatatatatatatatatatatatattcaatttgctaatattttataagcagttgtcacatttatcttcatattgaggttcagttatatttttctttcctcatactgttcttgtctagattgtataccaaattgtaccagacctatatactatagagcgtttttctttttctactatttgaaacaatatagttagataagagaaatacctgttgttttaaggcttagtaaaacttcccctaaaactgtctaggtctggagtttttttatggaggtttttttaaaaaaaaaaaaagattcagtttctataatagttattgattattgaaattttccaaagtcaattttggtagtttgtcttctaaattgtgcattttatctattcaaattcattttcataaagtttttatttacttatctgtactttatctataattatataatttattaatttctaatattgttcttttttttgagacaggatctcaccctgtcacccaggatgaagtataatggatctagcttgatgcttaataaagtaatctatcctttgtttctggttgttttaaaattctttcccttggtatattatgggttcattagtataagttcaattttatatttatttatctcataatatcataacttggagcatactgtcactcagagaactttgtcattcatcaattctgggaatttcaaaactattgtcttcaaatattaccttcttggctggtaatttaccttcttggctcatgcctgtaatctcagcactttgggaggctgagttgggcgaattacttgagaccaggagttcgagacttgcctggctaacatggtgaaaccccatctctactaaaaatacaaaaattagctggctgtggtggcacatagctgtaatcccagctacttgggatactgaggcatgggaattgcttgaacccgggaggcagaggctgcagtgagccaagatcgtgccactgcactctagcctgggtaacagagtgagactctgtctcaaaaacaaaaacaaaaacaaacaaatattacattattgtattctcttgttttcaatatatattttcaaattacacagatgtttttctttttttttgagacagagtcttgctctgttgtccatgctggagtgcagtggcacgatctcagctcactgcaacctccacctcctgggttcaagcaattcccctgcctcagcctccttagtagctgggactacagatgtgcaccaccacaccaagctaatttttgtatttttagaagacacggggtttcaccatattggccacgctgatcttgaattcctgacctcaagcgatctacctgtcttggcctcccaaaatcctgggattacaggcgtgagccaccatacctggccaactttcttgtattctctttatcttcctcagctattttctgtataatatcctcagatctatcttctagtttataaattttcttcaaccatgactaattttatgttatacttgtccaagatgtttttaatttcagtgacaatatttttcattttgaaagttctgttttttggccaggcgctgtggctcatgcctgtaatcccagcactttggggggccgaggtgggtggatcacgaggtcaggagatggagaccatcctggctaacccggtgaaaccccgtctctactaaaagtacaaaaaattagctggacgtggtggtgggtgcctgtagtcccagctactcaggaggctgaggcaggagaatggcgtgaacccgggaggcagagcttgcagtgagccgagatcgcgccactacactccagcctaggcgacagagcaagatgctgtctcaaaaaaaaaaaaaaaaaaaagaactgtttttttttcacacctctctgttctttgtcactagcttcttagttttcattattgatgttagcctgtctttactggctttaagtaagtatgtttaaaatgatcaatctctttcagattgttgtgctatttcaaagtcttggggagccagtcgtcttatttgttgtgtctgctgattgttcctcatactggattgtttcctcttgtgaattgtgcttttttgcctgaccactaatcttctgcaaaaacttcttagtgtgtatgtatgaggaaatatcatatattcttatttgcaaaagacttgttgatgaggaatacctggagcttccatttcttacaagagacattttttccaagcttcactttctaagattagtgagcagagatcgtataagcatccttttcactaagagtgatgcccactgggcacggtggctcatgcctgtaatcccagcactttgggaggccaaggtgggtggctcacgagatcaggagttcgagaccagcctggccaacatgatgaaacccgtgtctactaaaaatacaaaaaaattagctgggcatggtggtgcacgtctgtaattccagctactcaggaggctgaggcaggagaatcgcttgaacccgggaggtcctacccttctatgattctagtttcatttccacctccccacctctgtacctatatcagcattaaatttccagtctccagccattgatattaatctatttgcatctaaaatctttccaaacagctttgtgtcagcttttgagatgaccctggctttaagttcgatgatgaagcatttcctttacttacttttgaaactgaactatatattgaaccttttgtttatttattttttatcaagaatttgtatgtgttcataacattttaccttttcataaaattgcatgtaaaactttgtagtaaaagttttaacctgtgttgttacaaacagcaaatttcttttttatgttttgcaatttgcaaatggaaaatagaagagtttgtaaattagatatatgagtttggaaattatatatcttatttttactgtaactagtggtcactgtaatattttttataaacacataaagatttgtttttctgtagcaatatagttttttttagttatcctcatgggataatagtgatgatttcctgcatgttctatatcgtattaatcattagtattattagtaatctgaagtaaaatgaagtatgcttgatttttactttctattctattttccttctttttttccttttcctttttttttttttttttttttttttagtgagacatgctctgttgtccaggctggagtgcagtggcgtgatcttggctcactgcagcctctgcctcccaggttgaagcgattctcctgcctcagcttcccaaatagctgggattacaggtgcctgccaccacccctgactaattttttgtaattttagtagagatgaggtttcaccatgttggccaagctggtctccaactcctgacctcaagtgatccacccgcctcagcatcccgaagtgctaggattacaggtgtgagccgctgcgcccagacctattttccttcttttgtattactatttaaaaatattatttacaggctgggtgtggtggcttacacctgtaatctcagcactttgagaggccaagatgggaggattgcttgagcccaggagttcaagagcagcctgggcaacaaaacaagaccccatctctacaaaaaagcatacaaacaaaaaattagccatgtatggtggtgggcacctgtagtgcctgctcttgaaaggctgagatgggaggattttttgagccaaggggttcaaagctgcagtgtgctgtgattgcatcactgcactccagcctcggtgacggtaataaccctgtctcaaaaaaaaaaaaagatattattcacaaaatttttagtgtgttctccactgctttagaaaacccattctttttcattttgctctcaaattccccactcctggtttcttaatgttatttctctgtgttctggaacatttatttgagtaatgtattcacaaaagatgactaggtagtgttggctgagttcttacatacctagaaatgtcttttgccattatgagtgagcaacagttaacttggatataaaattattgggatcttgtattaggtaggttcaagccgagcattgtaaaataaagccccaaatgtacagatcaaataaagataaaaatttagttctctttaatgtaatactttgttatgcaacagtgctcaacgactgtgttctacaaggccatcctggctgatgggatcacctctgttatcacattatttggctttcatctttgagtctaagagcgctccttccccccttgccccagtcctttgaaatgtctagtcaagaccctttcctagttttcgtttgtttgtttgttggagacggagtctcactctgttgcccaggctggagtgtatggcacgatctcggctaactgcaacctccacctcccaggttcaagcaagtctcctgtcttagcctcccaagtagctgggattataggtatgtgccacaggcctggctaatttttatatttttagtagagacagggtttcatcatgttggccaggctggtcttgaactcctgacctcaggtgattggcccgccttggcctcccaaagtgctgtgattacagacatgagccactgcgcccgtccttgccagttctcattagttctctttgatggctaaaacctcttacactattataagaaagcatataggagataaagtatgtaggagatactgctatagagtttaaggagatagagatggtattggcagtggtggttttgcctatgtgcccttctctagagttgatgtagagttgcctagagtatcctcccttagagttgatgtgaattaaacactgtggttttacaaagagaaccccagaaaagtagctgaggccattgataaagagcaggtaggtcattagctgctatattatcattgctaaattttgcattcttttcttgcagaaaatatatttgatttatttgtttaggatttgggggtgagctttgatgtgtaattactttatcagttgattgttaactagaaaatgagaaataagtgacttgtttgctaccattgtttttgaagggagaaattcagaaaatatatttactgagaagttaaaggaagacagtattcctgctcctcgctctgttggcagtattaaaatcaactttacccctcgagtattcccaacagctcttcgtgaatcacaagtagcagaagaggaggaggtatatatacttactctagttacaattaccacaaatgaaaatactttatttttatttcatctattgaaattagtactggttctgaatttcttaagtatttattacactaaagaaataacgtcatgaatatgttttagaatttccttatgagaaaatatattttgcagatgtttttggctccagaatagtgataagagaataccgacttaatatatgtacagtaggccgggcgcagtggctcatgcctgcaatcccagcactttgggagcccgagatgggtggatcacttgaggtcaggagttcaagaccaacctgactaacatggggaaactccatctgtactaaaaatacaaaaattagcctggcgtggtggcgagcacctgtgatcgcagctactagcggggctggggcaggagaattgcttgaacttgggatgtggaggttgcagtgagccgagattgcaccattgtactccagcctgg

SEQ ID NO: 8 - DNA, 50000 bp, Homo sapiens
  human DYXC1 chromosomal gene region, nucleotides 100001-150000
gcaacagagtgagactttgaatcaaaaaaaaaaaaaaaaaaaagcggtctttttgttgttgttgtttgttttagagatggagtttttgctcttgttgcccaggctggagtgcagtgggcacaatctcagttcactgcaacctcctcctcccaggttcaaacaattctcctgcctcagcctccctagtagctgggattacaggcatgcgccagcatgtctggctaattttttatatttttagtagagatggggtttcaccatgttggtcaggctggtctcaaactcctgacctcaggcgatccaccctgctcggcctcccaatgtgctgggattataggcatgagccaccgtacccagccaagaactgcatttttttactgcgaggctgaattgatgcatctttgatttgaaaaattgtagaaataagaagatccttaagacataatgacaagccgggcgtggtggctcacgcctgtaattctagcactttgggaggccgaggtgggtggatcacctgaggtcaggagtttgagaccagcctggccaacatggtgaaaccccatcgctattaaaaatacaaaaattagtccagtgtggtggcaggcacctgtaatcccagctactcgggaggctgaggcaggagaatcacttgaacccgggggtcagaggttgcaatgagccaagattgcaccatttcactccagcctgggtgaaagagtgaaactctgtctcaaaaaaaaaaataaaaaataaaagacataatgacaatataaataattatttcatattattaaacaaggtattttctatatttgtaacatttctataattatacagttataataataataataattattattattttgtgggatggagtctcgctctgtcccctaggctggagtgcagtggcgcagtctccgctcactgcaagctccacctcctgggttcatgccattctcttgcctcagcctcccgagtagctgggactacaggtgcccaccgccacgcccagctaattttgtgtagtttttaatagagatggggtttcaccatattagccaggatggtctcaatctcctgaacttgtgatccacccacctcggcctcccaaagtgctgggattacaggtgtgagccactgcgcccggcccatagaattatttttgaattgtgtttatacacacacacacacacacacacacacacacacacacacaccctttttttttttttttttgagttgggggtcttgctctgctgcccaggctggagtacagtgggacaatcatggctcactgtagccttgaactcttgggctcaagtgaccttctgcatatttttcagtacagaaaaatgaaaatataaaagaattttagatcaatatcctggatgaacatcgatgcaaaaatcctcaataaaatgctggcaaaccgaatgcagcagcacatcaaaaagcttatccaccatgatcaaatgggcttcatccctgggatgcaaggctggttcaacatacgcaaatcagtaaacgtaatccagcatataaacagaaccaaagataaaaaccgcatgattatctcaccagatgcagaaaaggcctttgacaaaattcaacagcacttcatgctaaaaactctcagtaagctagatattgatgggaagtatctcaaaataataagagctatttatgacaaacccacagccaatatcatactgaatgggcaaaaactggaagcattctctttgaaaactggcacaagacagggatgccctctctcaccactcctattcaacacagcatagtgttggaagttctggccagggcagtcaggcaggagaaagaaataaaggggctcccctctcccctctcccctctcccctctcccctctgccctctcccctctcccctctgccctctcccctctcccctctgccctctcccctctcccctctcccctctctctttccacggtctccctctgttgctgaggctggactgtactgccgtgatctcggctcgctgccacctccctgcctcaggctcctgtcattctcctgcctcggcctgccgactgcttgggattgcaggcacgcaccgccatgcctgactggtttttgtatttttggtggagatggggttttgctctgttggcccggctggtctccagctcctgacctccagctcctgatctgcccgcctcggcctcccgaggtgctgggattgcagatggagtcttgctcactcaatgctcaatgttgcccagactggagtgcagtggcatgatctcggctcgctacaacctccacctcccagccgcctgccttggcctcccaaagtgctaagattacagcctctgcctggccgccaccccatctaggaagtgaggaacatttctgcctggccgcccatcgtctgggatgtgaggagtgcctctgcccagccgcccagtctgggatgtgaggagcacctctgcccgaccgccaccccatctgggaagtggggagcgcctctgcccggctgccccgtctgggaagtgaggagcgcctctgcccggccgcccatcatctgggatgtgaggagcgcctctgcctggccaccccgtctgggaagtgaggagtgcctctgcgcagccgccccatctgggaagtgaggagcacctctgcccgtcagctgccccatctgggaagtgaggagcgcctctgcccggctgccccgtctgggaagtggagagcgcctctgcccagctgccccgtctgggaagtggggagcgcctctgcccggccgcccatcatctgggatgtgaggagcgcctctgcctggccgccaccccgtctgggaagtggggagcacctctgcccagccggcccgtctgggatgtgaggagcacctctgcccggccaccccatcagggatgtgaggagcgcctctgcccggccgccccatctgggatgtgaggagcgcctctgcctggccaccaccctgtctgggatgtgaggagtgtctctgcccggctgccccgtctaggaagtgaggagcgcctctgcccggccaccccgtctgggatgtgaggagcgcctctgcccggccgccaccccatctgggaagcagggtgcgcctctgctcggccgccctgtctggaaggtgaggagcgcctctgcctggctgcccttcatctgggatgtgaggagcgcctctgcccggccacccggtctgggaagtgaggagcacctctgcctggccaccccgtctaggatgtgaggagcacctctgcccgaccgccaccccatctgggaagtggggagcgcctctgcccggccgccccgtctgggaagtgaggagcgcctctgcccggccgcccatcatctgggatgtgaggagtgcctctgcctggccaccccgtctgggaagtgaggagtgcctctgcccagctgccccccccccccgtctgggatgtgaggagcgcctctgcccggccgccccgtctaggaggtgaggagcgcctctgcccagctgccaccccttctgggaagtggggagcgcctctgcccggccaccccgtctgggaggtgaggagcgcctctgcccggctgccgcatctgggaagtgaggagcgcctctgcctggctgccctgtctgggatgtgaggagcgcctctgcccggccgccaccccatctgggaagcggggagtgcctctgcccggccaccccgtctgggaggtgaggagcgcctctgcccggccaccccgtctgggatgtgaggagcgcctctgcccggccgccccgtctgggaggtgaggagcgcctctgctcggccgccccgtctgggaggtgaggagcacctctgcctggctgccgcatctgggaagtgaggagcgcctctgcccggctgccccgtctgggatgtgaggagcgcctctgcccggccgccaccccatctgggaagcggggagcgcctctgcccggccaccccgtctgggaagtggggagcgcctcttcccggccgccccgtctgggaggtgaggagtgcctctgcctggtgcccttcgtttgggaggtgaggagcgcctctgcctggccaccccgtctgggaggtgaggagtgcctctgcctggccgccccatctgggaggcgaggagcgcctctgcctggccgccccatctgggaggtgtacccaacagctccgaagagacagcgaccatcgagaacgggccatgatgacgatggcggttttgtggaaaagaaaatggggaaatttggggaaaagaaagagagatcagattgttactgtgtctgtgtagaaagaagtagacataggagactccattttgttctgtactaagaaaaattcttctgcatgggatgctgttaatctataaccttacccccaaccccgtgcactctgaaacatgtgctgtgtcaactcagggttaaatggattaagggcggtgcaagatgtgctttgttaaacagacgcttgaaggcagcatgctcgttaagagtcatcaccactccctaatctcaagtacccagggacacaaacactgtggaaggctgcagggacctctgcctaggaaaaccagagacctttgttcatgtgtttatctgctgtccttctctccactattatcctatgaccctgccacatccccctctctgagaaacacccaagaatgatcaataaatactaaaaaaaaaaaaaaaaaaaaagggtattcaattaggaaaagaggaagtcaaattgtccctgtttgcagatgacatgattgtatatttagaaaaccccatcgtctcagcccaaaatctccttaagctgataagcaacttcagcaaagtctcaggatacaaaatcaatgtgcagaaatcacaagcattcttatacaccaataacagacaaacagagagccgaatcatgagtgaactcccattcacaattgcttcaaagagaaaaaaatacgtaggaatccaacttacaaggtatgtgaaggacctcttcaaggagaactacaaaccactgctcaacaaaataaaagaggacacaaacaaatggaagaacattccaagctcatggatagaaagaatcaatatcgtaaaaatggccgtgctgcccaaggtaatttatagattcaatgccatccccatcaagctaccagtgactttccccaaagaattggaaaacaaactactttaaagttcatatggaaccaaaaaagaacccacattgccaagacaatcctaagccaaaagaacaaagctggaggcatcatgccacctgacttcaaactacactacaaggctacagtaaccaaaacagcatggtactgatactgaaacagagatatagaccaatggaacagaacagagccctcagaaataataccacacatctacaaccatctgatctttgacaaacctgacaaaaacaagaaatggggaaaggattccctatttaataaatggtactgggaaaactggctagccatatgtagaaagctgaaactggatcccttccttacaccttatacaaaaattaattcaagatggatgaaagacttaaatgttagacctaaaaccataaaaaccctagaagaaaacctagacaataccattcaggacataggcatgggcaaggacttcatgtctaaaacaccaaaagcaatggcaacaaaagccaaaattgacaaatgggatctaattaaactaaagagcctctgcacagcaaaagaaactaccatcagagtgaacaggcaacctacagaatgggagaaaatttttgcaatctactcatctgacgaagggctaatatccagaatctacaaagaactcaaacaaacttacaagaaaaaaacaaccccatcgaaacatgggtgaaggatatgaacagacacttctcaaaagaagacatttatgcagccaacagatacatgaaaaaatgctcatcatcactggccatcagagaaatgcaaatcaaaaccacaatgagataccatctcacaccagctagaatggcgatcattaaaaagtcaggaaacaacaggtgctggagaggatgtggagaaataggaatacttttacactgttggtgggactgtaaactagttcaaccattgtggaagacagtgtggcgattcctcaaggatctagacctagaaataccatttgacccagccatcccattactgggtatatacccaaagattatcaatcatgctgctataaagacacatgcacatgtatgtttattgcagcactattcacaatagcaaagacttggaaccaacccaaatgtccatcaatgatagactggattaagaaaatgtggcagtaatgtgtgccatccagggctagagcaagtgcggccccacggcctgcacggccctgagccatgctgaggagctgcgccaggcgcctccgcacgctgggggccctttgccagccgccaccggtaggcaggcgcctgccgggtagtgagccgcaacccgcgctgaggtcattttcttctgaggaagtcattcttaaggactattctgtccccaaccccagctggaacaaggacctaagactgctctttgaccagtttatgaagaaatgtgaagacggctcctggaaacgtttgccttcatataaacgtacacctactgaaaggattcaagactttaaaacccattttcttgacccaaagcttatgaaagaagaacaagtgtcacaggcccagctcttcaccagaagctttgatgatggcctgggctttgaatacgtgatgttctacaatgacgttgagaaaaggatggtttgcttatttcaaggagtcccttacctagaaggaccacctggattgattcatggaggtgccattgcaaccatgattgatgctactgttggtatgtgtgcaatgatggctgggggaatcgtcatgactgccaatctcaacatcagttgtaaaagacctatccctctttgttctgttgttaggataaatagccaacttgataaagttgaaggaaagaaattttttgtttccagtaatgttcagagtgttgatgagaagaccctatattcagaggcgacaagcttacttataaagctgaatcctgctaaaagtctgacataaagagctgctggtgaactccatctcattctctcccctccagaagaagcagttgtcccccgaatactctgctccctcactgctgaatccctgtagggagaagcctgccaagagtgaccttccgaaacagccttctgaatacaaggaggattcagtttccatcttcccaactttttaacacagaaacacttcctgcgagcatttcgacaactctcagtcagggcgctgtggctcacgcctataatcccagcactttaggaggccaaggcgggtggattgcctgagctcaggagttcaagatcagtctggacaacacgatgaaaccccgtctctactaaaataaaaaaaattagccgggcatggtggcgtgtgcctgtagtcccagctactcaggaggctgaggcaggagaattgcttgaacccgggaagcggaggttgcagtgagccaagatcatgccacatcactccagccttggcaacagaacgagaacccatctcaaaacaaaacaaaacaaaaaaaactctgtctccttaggatgtgttacctgctccactgcggactggagaaataaatgtgtattgaaacttttcctgcaaacttgaaaatgctagaaaaccaagcatgaagaataaatacatggcagggaagacacagacatgtgcatatgcatatacattataagttagtaagttagaaagctagctttgttgtatctgaacacatgaatctttttggtcagagtaggaagcacagccaaattctataggaatgaatacctcaaatctgttgtctgggattctagagattaaaccactgtgccatttaaaaaagaaaaaagaaaatgtggcacatagacaccatggaatactatgcagccataaaaaaggatgagttcatgtcctttgtagggacatggatgaagctggaaaccatcattctcagcaaactattgcaaggacaaaaaaccaaacaccacatgttctcactcataggtgggaattgaacaatgagaacacttggacacaggaaggggaacatcacacaccggggcctgttgtggggtggtgggaggggggagggatagcattaggagatacacctaatgtaaatgacgagttaatgggtgcagcacaccaacatggcacatgtatacatatgtaacaaacctgcatgttttgcacatgtaccctagaacttaaagtatagtaaaaaaaaagaaaaatgaaaatattataacccagaaagataaaataataatcacatgggctgggcatggtggctcacgcctgtaatcccagcactttgggaggccgaggtgggcagatcacaaggtcgggagttcgagaccagcctggccaatatggtgaaaccccgtctctactaaaaatacaaaaactggccgggcatggtggcacacgcctgtaatcctagctgttcaggaggctgaggcaggagaattgcttgaacccagaaggcggaggttgcggtgagctgagatcatgcaactgcactccagcctgggggacagagtgcgactgtctcagaaaaaaaaaaattaataatatcatatgaactcccagaaagcaagaaatttggctagaaattttatctattgtatttccaccagctattgaggtttaaaaaaaagtttaaggacgggcacggtggctcaagcctgtaaccccagcactttgggaggccaaggtgggtgaatcacaaggtcaggagtttgagaccagcctggcccatatggtgaaaccccatctctactaaaaatacaaaaaattagctgggcatggtggcgtgtgcctgtagtcccagctatttgggaggctgaggcagaagaatcgcttgaacccaggaggcagagggtacagtgaaccgagattgtgctactgtactccagcctgggcagctgagtgagactctgtctcaaaaagaaaaaaagtttaataatgtataaatatggccaggtgcagtggcccaacgcctgtaatcccagcactttgggaggccgaggtgggcagatcacaaggtcaggagatcgagaccagggtgaaacctcgtctctactaaaaatacaaaaaattagctgggtgtggtggtgggtgcctgtagtcccagctacttgggaggctgaggcaggagaatggtgtgaacctgggaggcagagcttgcagtgagccgagatcgcaccactgcactccagcctgagtgacagagcaagactccgtctcaaaaatatatatatataaatattgcaagattaaaatcaccatcattggccaggcgtggtggctcacgcctataatcccagcactttgggattccgaggtgggtggatcacgaggtcatgtgttcaataccagcctggccaaaatagtgaaaccctgtctctactaaaaatacaaaaattagccaggcatggtggcacacgcctgtaatcccacctactcgggaggctgaggcaggagaatcacttgaacctgggaggtggaggttacagtgagctgagattgctccacttgcactccagcctgggtgacagagcaagataaaaaaaaaaaattaccaccatcaagtagtacgttaatctgctctggctgctataaacaaaataccatagactgggtggcttaaacaataaacagtcatttcttatagttctggaggctgggaaatccaagatcagggtgccagcatgattcttggtaggggtgctcatcataacttgcggacacccactttctcactgtgtccacatgggggagctctgatctctttctcttcttaaaaggataaactcgttttgggagctctcctctcatgacctaaacctcattacctctcagaggccccactaccaaataccgattcacattgatggttatggcttcagtatatcaatttggaggagaggtggagaagacacaaacactcagtgcatagcaaataatatttcaatccactggaggaagatggaaaatctaaaaggaaatgttaattaaaaaaagacctttttattcttcataagacttctttctcattctagtggctacacaaacaagctgaggcacgaagagcaatgaatactgacatagctgaactttgcgatttaaaagaagaagaaaagaacccagaatggttgaaggataaaggaaagtaagttgtttgaactctgatcatgagctttatcatgtatgaagacattagcagttatcataggacgagtatggtggctcatgcctgtaatcccagccctttgggaggccgaggtgggatgataacttgaagccagaagtttgagaccaacctgggcaacaaagcgagattttgtctctacaaaaaaacaaacaaaataaaacattagcctggtgtggtggtgagcacctgtagtcccagctactcagaaggctgcggcgggaggatcccccaagcccaggagttcatggctgcagtgagctatgattgtgccactgcactccagtctgggcaacaagagtgagaccctgtctttgaacaaacaaagtagttatcacattctctagatattttgtctgtttgtgtgttgttggagaaagataatgcaaataagcaaataaggtagacagggcatccatattttaaaagtcagataacaaggcaatgaggctctaattttatttttttatttaaatgtcataaaaatgaaaaaaattacatcttttggaatgcttattaaaattccttttaaaatccgttttacatttgacataagtcctttctgatgtctcccataaaaacgggctttttgcaaagctacagctgttttgattattcttatccattttttttcttttttttttttgtgagatagtgtctcattttatcacccaggctggagtgtagtggtgcaatctcagctcactgcaacctctacccgccaggttcaagcagttcttctgcctcagcctctcaagtagctgggattacaggcatccaccgccacacctggcgaattttttttgtatttttagtagagacggggtttcaccatgttggtcaagctggtcttgaactcctgacctctagtgattcatccacctcagtctcccaaagtgctgggattacaggcatgagccctggtgtgggacctattcaaatccatttctaaagattaccatcttactgccttccaggtcagtgatgtaaacacaaaaaagattaccatattatcccagaaattatcttatatctgggcagtcattttattcatttgtatatggattttttttttttttttgagacaaagtctcgctcttggtccccaggctggcgtgtgatggcacaatcttggctcaatgcaaactccacctcccaggttcaagcgattctcctgcctcagcctcctcagtagctgggattacaggcgcctgccaccacgcccaactaatttttgtttcaccatgttggccaggctggtctcaaactcctgacctcaggtgatccgcgcaccttgacctcccaaagtgctgggattacgggtgtgagccactgcacccggccgtgaatattttaacatactgatcaaaaacagtgaattctggcatattctgagtggcaatttgtgttcatattttgaggcggctatagatattatttattgtatttcatcatatacacttctgtgtttattttcctattacttattacttctataacaaactaccaccaagtggcttaaaacaacacaaatttattatcttatagttctgtaattcagaagtccaaaatgagtttcactgggccaaaattaggtgtcaactgccttccttttgaaggctctaggagaaaagctgttttcttaaatttccggcttctaagctgtttgcattccttgaattgtggctccccagccactgattgcgttattctgacctctgcttccgtgatctccttttctgactctcctgcctccttctaccttttataaggcctcttgtgtttatgtaagtcccacctacatcatccagggtactctctccatcaaactctttaacttattcacatgtgcaaagtcctttttgccgtgttaggtaacgttcataggttctgggattagaatatggacatctttttttggggggcggggggcattatctgtctaccacaatatctatatttttcctttctttctactttttcttggtcttttctcttgtctgttacaacaccagggattgttttgtggcgtttagctggagttggcaatttttctgaaggttagaagcctggaactgacaggttggttcttctaactcccaagagtaaaaccatatataatctttcctcctctttctcttcttcctttcttccttcctattcatttattcagccattaaataagcaattattgattgcttgctatttgccagactataggcacttaacaacagaaatcaggaggagagagacgtacacaaatatttaaaacactgtgtgggaagtacaataacatggataagaacaaggtaatagagccaggtgcggtggctcacgcctgtaatcccagcactttgggaggctgaggcgggcggatcacctgaggttgggagttcaccagactgaacagcaaggagaaaccctgtctttactaaaagtacaaaattagctgggtgtggtggcgcatacctgtaatcccagctactcgggaggctgaggcaggagaattgcttgaacctgggaggtggaggttgcggtgaatcgagatcgtgccactgcactccagcctgggcgacagagtgagactccatctcaagataaaaagaaaatttctggccaggcacagtggctcacgcctgtaatcccagcactttgagaggccaagatgggtagatacttgagttcaggattttgagacaagcctggccaatatggtgaaacccaatttctactaaaaatgtaaaaattggctgggcacggtggctcactcctataatcccagcactctgggaggccaaggtgggtggattacctgaggccaggagtttgagaccagcctggccatcatggtgaaaccctgtctctactaaaaatacaaaaactagtcaggcattgtggcgcacacctgtaatcccagctaatctgagtctcagaggcatgagaatcacttaaacctgggaggtagaggttgcagtgagctgagatggcaccactgaactccattctgggcaatttagcaagactgctcaaaaaaataataataattaaaaaaataataataatgggcaaagaacttaaataggcatttctccaagaagagagacaaatggctagtaaatataaaaagatgtttaacatcactaaccatttgggaaatgtcttatcaaaaccactatgagatgccacttcacacccattaggatggctactcagaaaaaaaacagaaaataacaagtgttagtgaggatgtggagaaattagaatcctgtgcactgttggtagaggtgttgaatgatccagctgctatggaaaacactattgtggttcccaaaaaagtaaaaatagggccgggcgcaatggctcacgcctgtaatcccagcgctttgggaggccgaagcgggcagatcacctgagatcgggagttcgagaccagtctgaccaacatggagaaaccccatctctagtaaaaatacaaaattagccaggcgtggtggtacatgcctgtaatcccagctactcaggaggctgacgcaggagaatcgtttgaacctgggaggcagaggttgcggtgagccaagatcgcaccattgcactccagcctgggcaacaagagtgaaactctgtctcaaaaaacaaacaaacaaaaaaaagcaaagactttgcattttgaagaaatcctttaatcgtgtacatctctttatttgaagagaattttggaacatttaaactgaaagagtatatccattgcaagtaaccaaaaaacccaatttttttatattgaaaggaatttattgtcttatataactaaacagctaaaggtgttttctatttcaagtaaagcttgatccagcagcttaactgtatcatgaagggctcggtttctttctgcctttttattcctttcatatttccatagtatcagctttattctaatgccagctcttcccacggtcttaaaaacggtcagcaacaatgtgggctatgtgacttcttcacttatatccagcagggggagaaatttgcccaacttttcattcttcaactttgaaaccaacccacaggcttcatcctaagtagaacaatataggtcacactagcccatccctgagtcaattactggagcaaaaataatgcaaatatgccctactggcttaaattactaatgtctacccttggaggtgggagtgaggtccattccattcaaatgacaggctggtatatgtgaaaagagatggattcagtgcaggcaacgtttaccctaaagagaatgcaaaaagaggtaagccatcctctttgtcaaatacaattagatattatttctgtgtttctgtttcaatgttaaattgtttttcttctctgtttttgtctcattagcaaattgtttgcaacggaaaactatttggcagctatcaatgcatataatttagccataagactaaataataagatgccactattgtatttgaaccgggctgcttgccacctaaaactaaaaaacttacacaaggctattgaagattcttctaaggtatggatatctatgtatgatatatggtgctttaaatatatccttctattaattggtttaaatgatgatctttttctgttcagttgaaataattgagatgcaaagattttaaagattgtctttcttattgaatctttttttttttaactgattttttactaatggacaaggaactggtgtttttttgtgacttctatgaatagttagttagattaaaaatcagctgttactggccgggcgcagtgggtctcgctatattgcacaggcaggtctcgaactcctgggctcaagctatcctcccgcctctgcctccctgagagctgggattacagacatgagccaccgcgcccggcacccagcccagatatctctacaatcattttctcatttgcagggtaacttagtatattctactttccattactaatatttactgtgtatgtcttgaacttctacagctttttcttttttccatgtcaagataacttgttttaagggcatatcatgtgcaaaatctcttaggtagcttcttgagattgaggatatggaggacacaaccaagtcttcatcactttgatatcatactacttttatgatccagtttttctttgactctcacaatgcaaacaaatatttgttgaatgaatgaataagctgaatcacccaatctgaatacaaaagcccacaaatgctatttaataattcctactggtaaaaataaagctgtggagatgtgaaatgataagctgaataataaccttggaggggtatgaatcccatataggaacaactgaaaaattccttaagttgttcaacatatctcttctggaaaacctgataataattatttgttttatatatatatataatatatattatatataagaatatatataatatatataatattatatatattatatataatatataaaatatattatatatattatatatattatatatattttatataatataatatatataattatatataatatatataatatatatataatatatgtaatatatataatatatattataatatatataatatataatatatgtaatatatataatatatattatatatataaaacaaatatatatatttgttttataaaaacaattatatatatttatatataaaaaacaaataattatatatatatttgttttatatatataatatatttatatttgctaaattcctgtaccagctaagctaaaaatttaggtgagtgcttgaggccatttttttttttttttttttgagacgaggtcttgctctgttgcccaggctggagtacaggggcacaatttcggctcactgcaagctccgcctccccggttcacgccattctcctgcctcagcgtccggagtagctgggactgaaggcgcccgccatcatacccggctaattttttgtatttttagtagagacggggtttcaccgtgttagccaggatggtctcgatctcctgacttcgtaatccgcccgcctgggcctccgcccgcctgggcctcccaaagtgctgggattacaggcatgagccaccacacccggccgaggccttttgtgtgtgtgtgtgtttgtgtgagacaaagtctcactctgtcaccaggctggagtgcagtggtgcgttcttggcttattgcaacccctgcctcccggattcaagtgattctcctgcctcagcctcctgagtagctgggattacaggtgcccgctaccacatctggctagtttaagtatttttagtagagacagggttttgccatgttggccaggctggcctcaagctcctgactttgggtgatctgcccacctcagcctccgaaagtgctggaattacaggcgtgagccaccaggcccacccgaggcttattttaaaattagaatttttaccatgtttttgcttgtttgttttttgtttttgttttttttgagacggattctcgctctgtcacccaggctggagttcagtggcgcgatctcggctctctgcaagctctgcctccctggttcaagcaattctcccgcctcagcctcccaagtagctaggactacaggtgtgcaccaccacagccagataatttttgtatttttttttagtagagacggggtttcgccatgttggccaggctggtctcaaaatcctgacttcaggtgatccacctacctcggcctccccaagtgttgggattacaggcgtgagccaccatgcccagccttttgcttgtttaaattaagaaactataactttcttgtaataattatatatattttgtaaggcactggaattattgatgccacctgttacagacaatgctaatgcaagaatgaaggcacatgtacgacgtggaacagcattctgtcaactagaattgtatgtagaaggtaaggaattgatagaaatggtttaagtcccaagttatactctgaaaaaaatgtcattggaattatttaagatcgtgacttttaaggactccatttttaaatatttttagcatttaatttattcttagtaatgtctgcagtatatctgacaatacatgatttctaaattatatttaaaaaattcattgtagaagagaattaaaataagtgtaaacaacaagtttcagtctagaaaaatgctatattagctgggtgcagatgctcatgcctgtaatcccagcactttgggaggcagaggcgggtggatcacctgaggtcgggagttcgagaccagcctgaccaacatggagaaaccctctactaaaaacacaaaattagcttggcgtagtggcacatgcctgtaatcccagctacttgggaggctaaggcaggagaatcccttgaacccgggaggcggagattgtggtgagccgagatcatgtcattgcactccagcctgggcaacaacagcgaaactccttctcaaaaaaaaaaaaaatgctatattaccagacaactcttcatagaaacgtgtgataaatgctgggtgtggtgcttcacgcctataatcccaggaactggggaggctgaggtggaaggatggcttgaggccaggagtttgagaccagcttgggcaacatagtgagaccctgtctcttaaaaaaaaaaaatacatgataggctgggtgtggtggcttatgcctgtaatctcagcactttgggaggccgaagcaggtggatcacaaggtcaggagatcaagaccatccgggctaacacggtgaaaccccgtctctactaaaaatacaaaaacaaaattagccaggtgtggtggcgggcgcctgtagtcccagctactcaggagactgaggccagagaatggcgtgaacccggaaggcagagcttgcagtgagccaagatcacgccactgcgctacagcctgggtgacagagcaagactctgtctccaaaaaaaaaaaaaaaaagataaaatatataaatgatttgagatatcatggatatttatctcagagagcttcttcggtcatccctgtatgtatgtatgtatttgtgtgtgtgtgtgtgtgtgtgtgtgtgtgtatacacacacctaagctcggaaataactgatccaccatagtgcatgcacattgcttatatttgttagattgtattaagttctgtaatttagctctatgtatacagaaaatatagcatttattttattttattttagatagatttaggggccgggcacggtggctcacacctgtaatcccagcactttgggaggccaaggtgggtggatcatgaggtcaggagatcgaggccctactggctaacacggtgaaaccccatctctactaaaaatacaaaaaattagccaggtgtggtggcaggtgcctgtagtcccagctactcaggaggctgaggcagaagaatggtgtgaacccaggaggcagagcttgcagtgagccgagatcatgccactgcactcccgcctgggcaacagaaggagactctgtctcaaaaaaaaaagatgggaatctaatttaaaacttggtacttgttctgaaccatgctactagtgattatttttattttaaacaactattgtctagataaaagaagtgctaataaaatatttattgtattaatcatcattgctatataaacacaggcctacaggattatgaagcggcacttaagattgatccatccaacaaaattgtacaaattgatgctgagaagattcggaatgtaattcaaggaacagaactaaaatcttaatgactattagaagtaactaagtattgttataagttttttaaaaacaactggaggcatctttgtacatattatggccagttgtacagaatcgctttctgtttagtactttagttctgttgagggcaaaatattataaatctatagaaaataaactgtttgacttgaatcatttctgaataagtaaatctaaataagaatctattttaattccttatttcttcatattaatacatatgtatacttttttgtgttactgaattaagcttgcccttgtaacaaaatatgttttggtatagttaccaggacacttactgattaatttttaacaaggtagaattttaaaataaaagatttataaataacttttgtttgtattaatataccatttaattgactgaatggttgactttggtataataaatattgacaggttgtccgttagtttccaactgcacagaaccttatttttttgtttaatacttctctaatgcttgttgttcaagattagaccagttcagcctagtataaaacaactgtttagaaaaccttatattcaacttcatttataggctgtgcacctgaaaactgatcatatttcataacactatctggtgaagataaatttttgagtgaaatagttttctagtacctccaagtagacaagggggagaaaagacagcaaagtgtaagatattaacctggaatattatggtattggtattggactgcaccatcatttgtgttgtatcactgggattatgcaagtgttaagcacaatagcacagcttcccttttgcttgtagttactgtaattctctaattaaattttaaaggtttttttttttttttgagacggaggaagtctccctttgttgcccaggctggagtgcaatggcgcgatcttggctcactgcaagctctgcctcctgggttcaggccattctcctgccttagcctcccgagtagatgggactacaggcgcctgccaccacgcctgactaattttttgcatttttagtagagatggggtttcaccatgttagccaggatggtctcaatctcctgatttcgtgatccacccaccttggcctcccaaagtgctggattacaggtgtgagccaccgcgcccagcctttttttttttttttcttgagacacagtttcactcttgttgcccagactggagagcagtggcctgatcatagctcgctgcatcctccacctcccaggtttaagggattctcctgcctcaggcctcctgagtagctgagattacaggcaccaccacgcctgattaattttttgtatttttagtaaagacagggtttcatcatgttggccaggttggtctcgaactgctgacctcaggtgatccacccgcctcggcctcccaaagtgcagggattacaggcatgagccaccgcgcccggccttatttatttatttttgagatgtagtctggctctgtaacccaggctttagtgcagtggtgcaatctcggctcactgcaacctccgtctcctgggttcaagcaattctcctgcctcagccttccaagtagctgggattacaggtgcgtgccaccacatctggctaatttttgtatttttagtagagacggggttttaccatgttggccaggctggtctcgaactcctgacctcaagtggtctgcctgcctcagcctcccaaagtgctgggattataggcaagaccccccgcgcctggcctaacaaatttagtttaaaggtctaattggcctttatcagtgactcatgaatcaagcagcatcttatataaagatttagaagagacactgctagaggtggcagaataaaatggcctttgcaaaattatgattaagacagtgaaagaggctgggcgcagtggctcaagcctgtaatcccagcactttgggaggctgaggcaggcagatcacaaggtcaggagatcgagactatcctggctaacatggtgaaacccccctctccactaaaaatacaaaaatttagccaggcgtggtggcaggcgcctgtagtcccacctactcgggaggctgaggcaggagaatggcgagaactcgggaggcggaggttgcagtgagccgagattgtgccactgtactccaacctgggtgaaagagcaagactccatctcaaaaaaaaaaaaaaaaaaaaaaaaaaaagaaaaaaaagacagtgagagagatttaacttaactgaatttatcttgcttttaacctccaagctgtccttgttcattcctgggtgtaggctgaactaactttaaaaaacttagtgtatagtttaaacaaagacagtgacagtcctttcccaaagccgacctccttcttgcctggggacggaattgcctttgtaggactaacattagccacaagattaaaaattatagtttatgctgggcatggtggctcatgcccataatcccagcactttgggacactgaggcaggtgggtcacttgaggtcaggagttagagagcagcctggccaacatggtgaaaccctgtgtctactaaaaatacaaaaattagccgggcatggtggcaggtgcttgtaatccctagctacttgggaggctgaggcaggagaatcacttaaaactgggaggtgaaggttgcagtgagcggagattgggccattgcactacggcctgggtgacaggagtgaaactccatctccaaaaaaattacggtttagaagtcatactgctggaggccaggcgcggtggctcatgcctgcaatcccagcactttgggaggccgaggcgggtggatcacctgaggtcgggagttcgagaccagcctgaccaacatggagacaccctgtctgtactaaaaatacaaaattagctggacctggtggcgctttcctgtaatcccagctactcgggatgctgaggcaggagaatcccttgaacccgggaggcggaggttgcggtgagctgaggtggcgccattgcactccagcctggacaagaagagcgaaactccatctcaaaaaaaaaaagaagtcatactgctggaggctacaaaattctgaccctccctaaactgctcctaagatcagtgtttacaatattttgcagaccctgcacttaatgaatcagctggcaccacccagatcaataaactgactcatctaatcttgtggcccccccaaccaggaactaacttagcacaagaaaacagctccaatctttaatttcatctctaaccaatgagccactcctggctcaccagcttcacctcacccaccaagttgtctttaaaaactctgctccctggctgggcgcggtggctcacgcctgtaatcccagcactttgggaggcttaggcgagtggatcacgaggccaggagttcaagaacagcctggccaagatggtgaaaccccgtctctactaaaaaaacaaaaaaatcagccgggtgtggtggcgggcacctgtaatcccagccactcgggaggctgaggcagagaattgcttgaacccaggaggcggaggttgcagtgagctgagatcgggccactgcactccagcctgggcgacagagcgagactgtctcaaaacaaacaaacaaacaaaaaaaaacaaaaaaactctggtcttctgcacagctggttctgtgtgaattactctttctctgttgcaatttccgtgtcttaataaattggctctgtctaggcagcaggcagagtaaacccctttgggggttacaaaaacaattggtatttatagggtagctgaagcaggaacaagaaacagcataatacaaaaagtgggttggttgatgtaacttcagatcactttccttgcaagagttaaaacagtgaggacttccctatcctgccagctaaaaggggcctgtttgggtatttggctatcttctctctcatgatttatcagaagatcagataaacaactaaattttgggccaggctcagtggctcatgcctgtaattccagcactttgggaggctgagatgggcggatcacctgaggtcaggggttcgagaccagcctggccaatcccagctccactaaaaatacaaaaattagccggacatggtggcaagcacctgtaatcccagctactcaggagtctgaggcaggacaatgactgagacagtgaaagagagctaacctaacagactccatcttggttctaacctttaagctgtccttgttccttcctgtgcataggctgaactaactttgggaggaacttagtttatagattatagttggaaacaaagatgttaacaacctttctccaaaaccttttccttcctgcctggggattagactgcctttgtaggactaagaaattagccacaagattagaaattatggtttaggagtcatgccgttggaagttacaagattctgaccctcactaaactgctcctaagatcagtgcttgagatattttgcagaccctgcacttgctggatcagctggcaccacctagctcaataaactggctcatcccatcttgtgacttccacccaggaactgactcagtgtaagaggacagcttcaattcctattgatttcatctacctaaccaatcagcactcctggctcactggcttccccccacccaccaagttgtccttaaaaactctgattcccaaatgcttgggcagtctgatttgattagtaataaaacgctggtctcctgcatagctggctctgtgtgaattactctgtctctgttgcagttcccctgtcttgataaattggctctgtctaggcagcaggcaaggtcaacccattgggcagttacactagcgcaagaggttcagtccaaatcagtggccttccatacattttatttaacacatcccagaaagaggttgtgaattacatcaagcaaaagggttctgtcctacaccaggtttgggatgatttttattttccacacgttagctctttgctgacagaggtcaaaatattagtcctaatacagagatattgtctaatagtttagtgttatataaacctattctgtcttggttccctagtgtagaagaaaatcagagttgaacagtaaagtgaaaaaaaaaatgcttttactcagtaacttataataaggaaaaagagacctcagcatggaacgaggcttagttcttttcattcctgtgaggcctgagaataaaaaatgaaaaagaactaggctcagttccaaatatggcatggacaagtaggggtttatagccaaggagtaggttgggtaatgaaggtggtcagtggatggaaaattaccaagagaaaacattaggggttagaggaattctggttaaattgacctaacaggattcttgctgaagacaggccaggttgatcagttatcaccggggggatggtagggaatgaggaatctggtcaaatatcaagggtgatcagatattgatggtggggattctggctaaaggaacttggcaggattcttgccatgactgggctcttaagagcaaagtatgtggctgagcgtgatggctcacgcctgtaatcccagcactttgggaggccgaggtgggcggatcaactgaggtcaggagttcaagatcagcctgaccaacatggagaaaccccatctctactaacaatacaaaattagccaggtgtggtggcacatgcctgtaatcccagctactcaggaggctgaggcaggagaattgcttgaacctgggaggtgacggttgtggtgagccgagaatgcgccattgcactccagcagcctgggcaacaagagcaaaactccatctccaaaaaaaaagcaaggtacagccgggcacggggtctcacgcctgtattcccagcactttgggaggctgaggcaggtggatgatgaggtcaggagttggagaccagcctgaccaacatggtgaaaccctgtctctactaaaaatacaaaacttagctgggcatggtggcgcatgcctgtaatcccaggtactcaggaagctgaggcaggagaatcgcttgaacctgggagacagaggttgcagtgagctgagatcgtgccactgcactctagcctggataaagagcaaggtgcaaggatggggcctagtcaaaaaagtggttcacaggagcctatctagagtttggtcaaggagagacctcttaatcactagcaagccaactgctttccaacaaatttcatccatgtgggaaataagggcaaaggattttgcacatgccatctagggaaaaagtaggtgccttacttcattttccactgctgtatcagaataccacagactaggtaatttataaaggacagagatttatttggttcatggttctgcatgctgggcagtcaagattaagggactacatgcatctggtgaagaccttcttgctatatgataacatggcagaatgtatcacatagtaagacagcatgcaagaagaaaggaaaggaaaagggggctgaactcccctcttttataactaaacccagtcctgcaataactaactcacttctgtattaacaactttaatttattcaagagtatggtgtccccatgacctaatagtctcttttgtgtgtgtgacagagtcttgctttgtcacccaggctagagtgcagtagtgtgatgtaggcttactgcaacctccacctcctgggttcaagtgattctcctgcctcagcctcccaagtagctgagattacaggcatgcgccaccatgcccggctaattttggtatttttagtagagatggcttttcaccatgttgcccaggctggtctcaaaactcccgacctctggcgatttgcccaccttggcctcccaaaatgctgggattacaggtgtgagacactgtgcctggcctatttttgtattttttaatagagacagcatttcaccatattggccagcctggtctcgcactccttatcttaagtgatccaccggccttagcctcccaaagtgctgagattgcaggtgtgagccaccgcgctggctttactttagcttttttagtccatattaaggactaacatgactctctagttatcttcagctatatttacatctgattgctgtaggtcagcagttctcaaaatgtgttctaatcagaatacaaatactctggtcagcatggtggctcacacccataatcccaacactttgggaggtggtgggttatgcctataatcccagcacttgggaggaggtgggaggataacttgagcccaggagtccaagactaacctaagcaacatagcgagacccccatctctacaaaaaatagaaaaaaagttagccaggcatggtggtgcatgactgtagtcctagctacttgggaggctgaggttggaaaatcgcttgagccggggaagtcgaggttgaggttacagtgagctgtgcttgtgtcattgcgctccagcctgggtgacagagggagaccctgactcaaaaagaatacaaaatatgctatattatatcatatcttaaaaaaaaaaaaacttttggctctacatctagctattgccccatttatctgttcagtgtgcagcaaaactacttaaaattagttttgagtttttctgttctttcttattccaataaagttttcctctctaccgcttcttcttgtagagaggaatacatttatttatttatttatttatttatttttgagacggagttttgcttgtcacccaggctggagtgcaatgtcgtgatcttggctcattgcaaactctgcctcctgggttcaagcgattctcctgcctcagcctcccgagtagctgggattacaggcttccaccatcacacctggctaatttttgtatttttagtagagatggagtttcaccatgttggccaggttggtctcaaacgcctgacctcaggcgatctgcccacctcagcctcccaaagttctgggattacaggtgtgagccttcgagcctggccttgttgaaataattctgtttgaagtcatccatgacctctgatttgcctaatcaaaggtcagttctcaattcatatgctatatctcccaagataatttcacactgctgggtcattccctctccttgaaacaagctgggcttccaggcctcacagtcttctaatttcctctttcttcagtagtcactccttcttggtctcatttgtttcttcttgttctttgccttgacctctaaatggtggttttccctaaagcatagtcctcaggtctgttttctacctacactttgtagtcttatctggtttaatcattttaaataacatgttccaagtgataatcttcaatttgggctactcccttgaattcaagttgtatattcagttgccttacatcaacttgactttcctcagatgttctcttaacctcctttagagggtgtggtcagacatctccttatcagaaaccttccctttccacctacctaaagtaactccttgctgaagtactcactttcctcctaactctattttatgttttttcccgtaacacttatcacttggcatgttctatacatctagccatatatatttacatagttacctatgtatgttatcatgttatttatgcatatattcatgtgtatttttatttgtaaatatataaaaatatgtatttatacatgctttttggtctgttttctcctgctgggattcattagaatctaaactccattaggactagcttctagtgcctagtatagtcctgggacctttattattattattattattattattattattattattattattatacatttttttgagacggagtttccctcttgttgcccaggctggagtgcaatggtgtgatatcggctcaccgcaacctccgcctctggggttcaagagattcttctgcctcagactcccaagtagctgggattacaggcatgcgccaccatgccagctaattttttttgtatttttagtggagatggggtttctccatattggtcaggctggtcttgaactcctgaccttaggtgatctgcctgcctcagcctcccgaagtactgggattacaggcgtgagccaccatgcccagccagtcctgggactttaataggcactgaatgactgtgtatataatgaaattccagatgatatgcttgattttcagctacttatcctgataattttattattttttagatgaagtctccctctgtcactcaggttggagtgcagtggcatgatctcagctcactgcaacctccacctcccaggttaaagagattcttgtgcctcagcctctggagtagctgggattacaggcatgcaccaccatgcctggctaagcattttgtatttttagtagagatgtggtctcaccatgttgcccaggcttgccttgaactcctggcttcctgtaatcggaccacctcggcctctcaaaagtgctgggattttaagtgtgagccactgtgcctggagtatcctgagaatttttttttttttttttttgagatagtctcactctgttgcccaggctgcagtgcagtggtgtgatcttggctcactgcaacctctgcctcctgggttcaagcgattctcctgccgcagcctcctaagtagctgggactataggcacacaccaccacgcctggctaattttttgtatttttagtagagacggggttttgtcatgttggctaggctggtctcgacctgctgacctcaagtgatctgcccgtctcggcctcccaaaatgctgggattacaggtgtaagccaccgtgcccggccaatcctgagaatttagcaacaacaaaaatgaccgaacagtgcaccactgccataggtaggaattttaggttagtatctagattctacatataaaaaggtaaattcacttgccatgaattgggcacctgcagtgaagggggaaaatggtttgtatggtttatactgcaagaacaggctaataaactaaaatgttattgcataattcacttggacatcttgtgcagtgccccactatttgatgttttgaggaagtgaaaaaaattaatttttcttcttgcttgtgccattactaaatatcagttttgggggggtatttggaagaagttgatgagtctgcagagtttgagctgtttccttacatcaagcaaattttatgattagaagattcaacttttataatccccttatgactcacaaatttattctttctaattgatggtaatatcctgaactgaaaccaacaggggaaaataacaatgattttttttttttccagaaagatactcagacccaattatcaacatatggtttaaacagatggcagctgaaataaatctgtgtgtagtagaatcaaagattttaaagaattgtgtagcctgtcacctgccactgctcacactgtgttgaagatatataaaattaattgcatttataaaaatataccacattgtctcacatctttgtacttttatccatatagtttcctctccctagaatagccttcttttgatttagctttatcttctccaggacacttctcctgatctctctggtctcaatttgagcaattctgtgtctgtctgaaaggcctgtgcatactgcctactttacattacaaaaattatcacatattaatttgctaatctcattctcccaaaatacttttttttattctttttttttgagacagagttttgctcttgttgcccaggttggagtgcaatggcgtgatcttggctcactgcaacctccacctcccaggttcaagtgattctcctgcctcagcctcccaaagtagctgggattacaggcacccgccgccatgcccagctaatttttttgtatcttagttgagatggggtttcaccattttggccaggccggtcggtcttaaactcctgacctcaggtgatccaccagccccgggctcccaatgtgctgggattacaggtgtgagccaccgcacccggccaatattgttaattaaaacaaaaatcactgtcttttatttctgtatctctatttgcatagtacagtctgccatcagtcggggttcaaaaaatatttgctcaatctatcaataaattacaagttctctcttccaccagcctagcagtgggaactaacacaatgttaaaagtgggaaattggctgggcagtgtggctcactcctgtaatcccaacactttgggaggccgaggcaggatgactgcttgaggacaggagtttgagatcagcctgggcaacacagtgagactccatctacacacacacacacacacacaccccacaaattagctaggggcatggttgttggtgcctgtagtcccagctagtcgagaggctgaggtgggggatagtttgagcccaggagtccaaggttgcagtgaaccatgatcatgccactgcattccagcttgggtgagataaggtgaaaccatttctccaaaaataaaaaatataagtgtgttggctgggcacagtgactcacacctgtaatctcagcactttgggtggccgaggcaggcggatcaccaggtcaggaaattgagaccatcctagctaacagggtgaaaccccgtctctactaaaaatacaaaaaattagctgggtgtagtggcgagtgcctgtaatcccagctactcaggaggctgaggcaggaaaatcgcttgaacctgggaggcggaggtcgcagtgagcaaccactgcactctaacctgggcgacagagcgagactccaactcactcaaaaaaaaaaaaaaaagtgtgaaattgttggtcagaagcttccttaaatcaatttttttttccttacagctcctgtgttcctttgtcttgataagtatactcaatagataagtaaataaaatatgaaaggcttttgaagcttttggattacatggactaaaacactttctaccttttattttgctattttgaacatattttcatttaaaaagtcactgtcatgtagttatcagttgaaatactttatttaacttattttaaaaattgattcaaaactttttatcttctttattaggtattctacccttagatttaacactcatttttcccgagtatattttctcttcaatatacaaaggaatatattttcatagagtgtgttgaatattaggaaaatcaagagttctggttctgtcaggtgcagtatttagagtgttattttgataaaatccagttgctctgatatggcttgaaaatacttgctcctttggagtataattgcaggggaaaaactgtggaataggtagaaattcaccataatgacttgaattggttttatacataatcatttgaggttttgatagtgaggtggctgtctggagtgtctttggatccaacatacatgaaaacacaggattgccagggttcacacaaggaggcagttgttcagatttcatttctgtgtctgaatttgaaggtgaagtacttttcttatcctaaaagataaacacataaaatcattctccattataaagttctgaaaaaaaaaaaatttaattcagtcccacccacttccctttttcaggaattctgttctcttgagggcattgaatgtcaggcctctgagcccaagctaagccatcatatccctagtgacctgcacgtatacatccagatggcctgaagcaagtgaagaatcacaaaagaagtgaaaatggccggttcctgccttaactgatgacattaccttgtgaaattccttctcctggctcagaacctcccccactgagcaccttgtgacccctgcccctgcccgccacagaataaccccctttgactgtaattttccattacctacccaaatcctataaaacggccccacccctatctcccttcgctgactgtcttttcggactcagcccgcctccacccaggtgattaaaaagctttattgctcacacaaagcctgtttggtggtctcttctcttgacgttgaagactacccttaaaatcacatacacctgtacacagagcaaagttgtcaaaaatatgctaagttacctttttaaacattctgtactagaactatgagaatttatttaaaaatctgtaaatcatagggaaaaatctgtaaaagtcatagttagaagaggaagtacagcttgattttagagtgagaaaggaacaagataaacatccagggttagcctgggtatgtgggcagccattaaataactaattacatagctaactgaataattacaattgtgataactgctatgaagaaaaaaaaatacaagatattaatgaagacagaatgggggttcttatctagtgttgggggttagggaaagccattccactgaagtaccattcaatctaagattgactataaggcaaggaagtaaaagatagaagtggggacaaagtatggcaaaagtcaagtaaattagggtgaaaatatcatgagatcagaatagagagccaggggttagatctcttagggttttgttgactgtgtttaaaatctgaatctttattgtaaaagcaatgaaaaactattgaagggttttaaggagaggagtgacatgaggtaatataattctagtaaaattactttggctgttgttagggcctgtgtgaatgtggtgaggagctgcagtagtccatgtgagtaatgatgacaatatatagtaggtggtagcatggagatggagcaaatttgtttgttttgttttgttttgtgtgtttgttttttgagacagagtctcgctctgtcatccaggctggactgcaatggtgcagtctcggcttactgcaacctccgcctcccgggttcaagcaattctcctgcctcagcctcccaactagctgggattatgggagctcaccacgacaaccagctaatttttttttatttttagtagagacagggtttcaccatgttggccagactggtctcgaactcctgacctcaggtgatccacctaccttggcctcacaaagtgttgggattacaggtgtgagccaccgcgcccagcctatggagcaaatttgaaatatattttggaagtagaattcacaggatctcatgattgattggaggtgggtggtgagggaaaaggacagatctaggataacttccaggtttctggacaaacacttgggcggatggtggtgccatttactacaatgtactacaatgtaatatgtaaattaaatatatatatgtagttttatatatagatgatacataattttggcactgaaaagcactttgcattgatgatggcttgataaattaatataacaagtttcgtccaggcgcaatggctcacacctgtaaatcccagcactttgggaggccaaggtgggcggatcacgaggtcaagagattgagaccatcctggacaacatggtgaaaccccgtctctactaaaaatacaaaaattagctggttgtggtgggatgtgcctgtagtcccagctactcgggaggctgaggcaggagaggatcccttggacccatgaggcagaggttgcagtgagccaagatcacgccactgcactccagcctggtgacagagcaaaagactctatctctctctctctctatatatataacaatgtttaaaaaaaccaacaaactgcatgaaaaactctaaaaaatgtcatgactgaatcctgaaattctgattgtgaattctatcctaagaaactttattgtagaaaaatttaattcacagcattacttacaaaagaactgaaatgtgtactgatttagatggaagttaattttaattctaatgctaatgccaaaaatactaagggagaagaccacccctcatattgtcttatgcccaatttctgcctccaaagaaagaagaggtaaaaactaaaaggcagaaatgaaatccacaggcagacagcccggcactacaccctgggcctggtagttaaagatcgacccctgacctaatcggttatgttatctgtagaatacagacattttatggaaaagcattgtgaaaatccctgtcctgttcggttccattctaattactggtgcatgcagcccccagtcacgtacccgctgcttgctcaatcgatcaggactctctcacgcggacccccttagagttgtaagcccttaagagggacgggaattgctcactcagggagctaggttgttggagacgggattcttgccgaatggccgaataaagcccttccttcccttccctcttttttttttttttttttgagacgaagtttcactctcgtcccccaggctggagtgcaatggcaggatctcagctcactgcaacctccgccttccaggttcaagcgattgtcctgcctcagcctcccgagtagatgggattacaggcacctggctaatttttgtatttttagtggagacagggtttcaccatgttggccaggctggtctccaactcctgaccttaggtgatccacctgcctcggcctcccaaagtgccgggattacaggcgtgagccaccacgcccggccaagcccttccttctttaactcggtgtctgaggggttttgtctgcggcttgtcctgctacaatactaatagaagtacttgaaaatagcttattttcttaaatagttaagtttcttctgcaaccttacattgatgtaagataaacgttcacccagccctggtataattatgtcagaataattacaaactgggtattctctaaagtgctgttagtataaatagactttttgtttaataaatttcactttgagatggttcacctattaaaagttaatagatgaacaatcaatgttttgtttttcttttttttgagatggagtctcactctgttgcccaggctggagtgtagtggcgccatctcggctcactgcaacctccccatcttgggttcaagcaattctcatgcctcagcctcccaagcagctgggattacaggcgtgcgccaccatgcccagctaatttttgtattttagtagagatggggtttcaccatgttggccaggctggcctcggacttctgacctgaggtgatccacccgcctcaacctccaaaaatgatgggattacaggcgtgagccaccatgcccagctggaaaatctattaaaagttaatgacttcacttactaacttttttggaagacttacaatgactaatcctttataaatttaatttcatttgcatttctagttgggaaaattacaaatataagaacatttctaggccgagtgcggtggatcacctgaggtcaggagtctgagatcagcctggccaacatggtgaaaccctgcctctactaaaaatacaaaaaattagccaagcgtggtggcacacacctgtaatcccagctactcgggaggctgaggcaggggaattgcttcaacccgggaggccaaggttgcagtgatccgagattatgccactcactgactccagccggggcaacaagagtgaaactctgtctcaaaaaaaaaaaaaacaaaaaaaaaccaggcctggcgcggtggctcacgcttgtaatcccagcactttgggaagctgaggcgggcagatcacagggtcaggagatccagaccatcctggctaacgcggtgaaaccctgtctctactaaaaatacaaagaattagccgggcgtggtggcgggcgcctgtagtcccagctactcgggaggctgaggcaggagaatggcgtgaacctgggagatggagcttgccttgagccgagatcctgccactgcactccagcctgggcgacagagcgagactccgtctcaaaaaaaaaaaagcatttctctatgtaatatcttatatctactcatattttaaatgtacttagagctgaaaaataaaaaaagtcctaatgagttgaaatgaagctgatttaaaatgctttccccataaaaacagtctttttttttttttttttttgaaacggagttttgctcttgttgcccaggctggagtgcaatggcgcgatcttggctcactgcaacctctgcctcccaggttcaagcgattctcctgcctcagcctcccgagtagctgggattacaggcatgtgccacaccaggcccagctaattttgtatttttagtagagacagggtttctccatgttggtcaggctggtctcaaactcccgacctcaagtgatctgcctgccttggcctcccaaagtgctgggattacaggcgtgagacaccacggccggcccttaaaaacattcttaaagggcagcttttaattaactgttttggtgtggagaagggagaaagtttttctctgtcactcaggctagagtgcagtagtgatcgtagttcactgcagcctcaaactcctggattccagtgaccctcccaagtagctaggactacatgcacaccaccatacccagctaatttttataatttttttttttttttgagacggagtctcactcttttgcccaggccggagtgcagtggcgcgatcttggctcactgcaagctccacctcctgggtttacgccattctcctgcctcagcctcccgagtagctggaactacaggcgccagccaccgtgcccggctaattttttgtatttttagtagagatggggtttcaccgtgttagccaggatggtctcgatctcctgacctcgtgatccgcccgccttggcctcccaaagtgctgggattataggtgcgcaccaccgcgcccagccttaatttttataatttttttgtagggatgaggtcttgctatgttgcccaggctggtcttgaactcctggtctcaagcaatcctcctgccttggcttcccacagcactgggattacaggcataagctaccatatcaatccccagcttttaacttaaaagtactataaaaccatattcaatctactcttcatcaactttttcttttttttgagacagagatctcactttgttgcccaggctggagtgcactggctgttcacaaatgggataatagtgcactgcaacctggaactcctgggctgaagtgatcctcaggccttggcttctcaaggtgggacttcaggtacatgccacagtgtccagctttcagtcaattattaatgttctacctgaatagtgtccctactaaagtttttgaaagtatttttccatgaataaggggactgaaaatatgaatcaaatataaagcaatttaaacttggacaagtcacttagattctctgtgcctaaaatttgtttgtaaaacagtggagcctggattatttgacctctcagatctcttctaccaactccacagaatttattaaccttataaacttgaggcagaatcacagtgtagtatactatgttttcatgttttgctttattttaatgagaatcataagcgaaagagggctggatgtgatatattggaagagtagcacaaggcagcaactatgacctaggttctcatactttatcatttcacctttatttgtatattatctgtaaaactggtaaataaaaatctgccatagttcctatggttgtggagattaaatgaaagaaaatttgatgtaaatggtgaatcactatatgaatgggatcaatttttttttttttttttttttttttgtggagacaaagtctcactctgttgcccagtctggagtgcaatggtgtgatcttggctcactgcaacctctgcctcctgggttcaagcaattctcctgccttagcctcccaaatcgctgggcttaaaggcgtgcgccaccacgctcagctaaatttatttatttttttttgtgagaaggagttttgctcttgttgcccaggctggagggcaatggtgcaatctcagctcaccgcaacctccgccttctgggttcaagccattctcctgcctcagactcccgagtagctgtgattacaggcatgcaccaccatgcctggctaattgtgtatttttagtagagatggggtttctccatgttagtcaggttggtctcgaactcccaacctcaggtgatccgcccgcctcggcctcccaaagtgctggaattacaagcgtgagccaccgtgcctagcctaatttttgtatttttagtagagatggggtttcaccatgatggccaggctggttttggactcctgacctcaagtgatccacctgcctcggcttcccaaagtgttgggattacaggtgtgagccactgcgcctggccaaatggaatcaatttttatcactattttattttattttttgagatggagttttgctctgtcacctaagctggagtgcagtggcgcagtcgcagctcattgtaacctctgcgtcccaggttcaagtgattcccctgcctcagcttccagagtagctgggactacaggtgcctgccaacacacctggctaatttttgtgtttttagtagaaatggggtttcaccatgttggccaggctgttcccaaactcctgacctctgatgatccgcctacctcagctttccaaagtgctgggactacagtcgtgagccaccatgcctcaccaaattttattattttcttttttgcaggtttacctcaatgtatgatcttccagaaacagttctgaaacctacaggttaatgaccttgtattagcataatatggtttgctaatattcacttaatactcataagtttgtagttggcaaagctggaaatagaactcaggttcactgagtgtcatcaatgttattctcatcagatcttgctgtttattctttttgaaactgcttattaatttgctttaaatattaacaacacagatactggataatagtaaatcataagaattttaaaattttgatggtatctcaactgatcagcttttcttaaattttctaggtttcctcagcacaggggaaagctttataggtggaaggtggaggccctgtgacttgcgcagaaaagtaccactgtaatttacttttatgggcacctaagcaggttgcccagggcaacttgtagattttgttgttgttgttgttaatactatctccttcagtcactgagaactcttttttatttttttattttttgagatggagttttgctcttgttgcctaggctggagtgcagtggcgtgatcttggctcactgcaacctccgcctcccggattcaagcgattctcttgcctcagccacccgagtagctgggattataggcatgcgtcaccacgcccggctaattttgtgtttttagtagagatagtgtttctccatgttggtcaggctggtctcgaactcttgaactcaggcgattcgcctgcctcggcctcccagagtactgggccttaaatgctggaattataggcatgagccaccgcgcccagctgagaactcttcttgttatcaaatgaccctctttactactggttctagaatttcgttcagctttggctcaaaacataatgcagaaaacgaaatctagtaaaaacagaagacaaattagctgataaactttgttggcataattagtaccataaaatctggaaaagctaaatcttaaattaaaaattttttagatttccaggataattcacaaatgtttatagattcagaaacaaaaggtccattgctttatactttacatcatagacaaccactgcagttttctaatatgggtttcaccttcttcaaacctgatattcttaaaatgtggagttcaaactgactatagttaagaacgaaacaaatagccaatacaaagaataataaaaggatgagttcaaattatctgccacatgtcatcaacttttattaaatgataaaaaggttaattaaaattgtaaatgatttttcagaagaaattacatcttaaaattggcctgtgaaagtggcttgaaaggtataaaaatctaccagtagctggtattagctttcttagtaatttaattttcagttagagaaagccagaccactttgacctttcgaagtcctttggtatagatacatatacatttaaaaagataaatgaaaacaagccattgattaataatgcccatcgcagaagctgtaattataaaattaagattttttgatgtcactgttgatgttgaatatagttaatagaaaagcaaaaataaaaaataaaaaaggtctaattttgaggcactcttttgtctctgctcaggtccaccgtcttccttgctaatgtttacgtttattattaatttttccatttatttatttacttttaccacaattaggggacagagaatgaaacctaaactttttattgcttctgtaaagagtgaatataaatgtaagattttttttttttgagacggagttttgctcttgttgcccaggctgcagttcagtggcgcaatctcggctcactgcaacctctgcctcccaggttcaagcaattctgctgtctcagcctcccaagtagctgggattacaggcccgcgccacctctcctggctaattttttgtatttagtagagacggagtttcaccatgtaggtcaggctggtctcgaactcctgacctcaggtgatccacccgcttcggcctcccaaattgctgggattacaggcgttgagccccggcgcccggccaccaaatcctttttcaaaacaactcccttctttgacacagtgcaatttaaataagtatacaattaacaaagtacgtttttcttgttcaaaaccatcactgaacagtttgcaacagtagggtatgaatgcatacaatctcgatggtctttacaccttcagaggctattcggacttaaggaaacgtgcatttacaagtgtaaatgtaaaagctcagtagttgccttgtaaagtgcttttcggggttgtggggttgggggtggtaggagtaggtggtgcctcaaattatactagcgaataccaacattcccaaacatctagccctctgcctaaactcacccggttgcggtctgtcattttcgctcttctttgttaattttttcggcaaagtcagttttcatgcagcagatggcaagaaagccggtgaacgcctagggacgtggccttttaaccgcacccaattaaagcagcaaacagaagatgaatagctgggtggatcagtatgtgtaaacctatccgtttaatccggggagctgttttcccgggtgaaaccacgaattctttctggaggttgcaactcgtaaccatagcaactggaagtggtcgccggctcccggatgttgtggggatctgcggccgcctggcccagcttcccgcagccgcccggcggcggcctgcgctccacttcgggagtggtagttcttctcttcaccgctgccgggcaggaggcgcaggcgggactacgcgtcacggcatgcactgcggcgcccagccggtcaccgcctgccggctgcagacgcctgcgagcaggttgtttttataagaggcgtcattggcgcccgagctgtgaccgccgccactggggcagccagcacaatcgggcggaggtggcgctgccccttcaggtactgtactaggcgggcttagaccccgggagggggtttgtgccgactgtctgagtccctggccgacacgctcggctcagtagaggagcagtggcggggtccgcctcctaggctggggctgctcgggcggggacttgacgcctggtgtcagcggccgcacgggcgacctcctcgggccgtgcgcgagtgcacagctccggaggcccgagccgaccctggggcgtccggtccggtggtcttgcagcctccaaaccccgagtgctataccgaactgcgcgccaagggtgggagagctgacggcctgggccacccttcttccttcactgggcaggctttgaggtgcttgtcggtctggactgatgaaaatccatatggtaagatgttatgtgcgtcaccctgccgggtttaatgccagcctgtcttttgatgacatttgagcccccgtgatgtcaggaatagaaagattaaatattaaataatgcccggagacttccgctagcacgccagcctgatgatgatcaaatacctctccatatccttctccagttccagtggcctcctttagctttggtctccctaatcgatttgtcctcattagcagctgtcccatgcagtgccttgtacgcgttgtttatgtttagcactattgggagcgccaagtaaggtattttattgagtggtgaagtttgcactgttctgctctacttttattgaaaaacacagttgggcattcttgtagttagaatagctaactatatagtactatgaattttagatttgtctcctccaatctacaggatagtgcttgaaatatcgctattactgcatcgtatcttttaagattagaatcatattgggtcccacacgtctcaggggatatttatcacacacatccagtgtgaatggtttacttttattttctgagttcaggtcatatagcagaagatggagatggtgagggtattattttaattcatattttttttttttttgagagagggtttcgctcttgttgcccaggctggagtgcagtggcgcgatctcggctcactgcaacctctgccttccagtttcaaacgattctcctgcctcagcctcccgagtatctgggattacaggcgcccgccaccacgcccagctaaattttgtatttttagtagagacggcggggcgggggggggggtctcaacgtgtcggccaggctggtcttggactcctgacctcgtgatccgcccacttcggcctcccagagtgctgggattacaggcgtgacccaccgcgcgggcgttaattcataatttttaaccttttacgctgagggcggaatttcagtcaaaaaagctgattaaaaatcagcccaaatacaatttaaaggtgtgggaaatactccttcttagatgaaaacagtttagaaaatagtgtaattgcaattttgtaaaccatggaacaaaaagtgaacataacttcttttagaaatgacttaaagggtccagagcggtggctcaggcttgtaatcccagcactttgggaggcccatgcaggtggatcacctgaggtcaggagttcgagaccagcctggccgacacggtgaaaccccgtctctactaaaaatacaaaaattagcctgtaaacccagctacttgggaggctgaagcaggagaattacttgaacccgggaggcggaggttgcagtgagccacaagatctggccaccgcactccaacctgggtgacagagtgagattatctcaaaaaaacaaaaaaaaaaaaacaaaaaaaaaaaaagagaaaaagaaagaaaagaaataaaggatattcacagttataaaagtgtagatagtgaaactagaaatgatttttcttgtactttcctgtatttttcaagttttctgtagaaaaaacttttaaaaaaggatgcaagtattaacagcactttgagagtttctatatcatttttttcactttctagtggaaagccccacccttatgcaaaaaaaaaaaaaaagcagcaattccagttatggactctatgaacattactttaaaaagaaaataaaggcctagagcagtggctcatgcctgtaatcccaggactttgggaagccaaggcaggcggatcacttgaggtcaggacttcaaggccagcctggccaacgtggcaaaaccccgtctctactaaaaatacaaaaattagctgggcatggtggcggatgcctgttatctcagctattcaggaggctgaggcaggagaatcgcttgaacccgggaggcagaagttgcagtgagccgagatcatgccactgtactctagcctgggtgacagagtgagactgtctcaaaaacaccaaataagcaacaacagtaaaaaacacaggcaaagtccctgttgctgcatctgtagactatatgaatttaagtgtaaattatttggagagtagggacaataaatgaaatagttggactcgaagaaaataacagatccgacaatttgtagcatttgtagatctcattttattttttggagatgatgtcttgctctgtcacctagtctggagtgcagtggcctggacacgacttactgcagcctcctgggctcaagtgatcccaacacagcctcccacatagctggggctacaggcacatgtcaccaggcctaggcctggctattactttttttgagacagagtcttgctctattgtatggtggttcagtctcggctcactgcaacctctgcttcctagtagctgggattacagtgtgattcaagctgagttcaagtgtgattctcctgcctcagcctcccagtagctgtcacatgtcacagacatgtgacaccatgccccactaaatttttttttgagacggagtttcgctcttgttgcctagactggagtgctatggcaagatctcagctcaccacaacctccgcctcctggtttcaagtggttctcccgcctcaccctcccgagtagctgggattacaggcatgtgccaccatgcccggctaattttgcattttttagtagagacagagttttttccatgttggtcaggctggtctcaaactcccgacctcaggtggtccgcctgcctcagccttctaaagtgctgggattacagacgtgacccactgcccctgacctaatttttgtatttttagtagagactgggttttgccatgttagccgggctggtcatgaactcctggcctcaagtgatctgtccgccttggcctcccaaagtgctgggattacaggcacgtagctgggattacaggcacacaccaccatgcccggctaatttttgtatttttagtagagatggggtttcaccataatggccaggatggtctcgatctcctgaatgcgtgatccggttgcctgggcctcccgaagtgctggcattacaggcgtgagccaccgcacctggccctaattttaaaattttatgtagaggcagggtctcgctatgtttcccaggctggtctctttgaactcctgggttcaagcgattttcctgccttagcctcccagagtgctggatacaggcatgagagctccatgtctggcctgtacatcccatcttttgatctggtttcttttcactttgtacagttaaatcagaggtgaaagtacttagcttttcttcacttaagcctggaaaatcttaaacttgtcaacattgaatcacgttcagaaccatgttttatatgtctgatattttggcttatgtctatttttgtttttattttttgttatggttatgttggatatggactggtaagtagtataatcaatcattagcccacattttgctttctgttgtaaagggctacattgttcttccacagtaatatacagtacatttttcttttttttggctactatcagcccttaaaaattaagggggtttaccttaacaccaagtgtccattaacactggataaaatatctattctaaagtgttttacaaaaaacactggtaacacgttgtttttcaggttgtggtgttcatactttattttcttaggttgtcttctttggagcttgcctactccaaatgtcaatttcttttcaatattttttaccttataattttgtatattctgtgtactgagttggctcaatatgttattaggctttgggtctattcacagggcataagaaaaattcataattgattaacatgtctgatttactaactcattccacagtattattgtatgggttgtataattatattcttatgcaagttaaagaaagacttttaaaaattaggtgcatatgaactgctaaaatcatggaagtagtagtttttgaacatgttaagaacatgaacacagcagcaaggcctgttaagtccaaggtactttaaacaaaaaatgcagactcatctaacagtgcagaactgctggaggctgaattatgtttgtgtttctttgtggtgaagatgaggaagttgcagtattgtaagtgataaaggattacattttcacaaatttccttcctgacagttccataattgtaactagactaaaacacttggtttcaaaggtcatttgcaatgtattgcatcagataaacttggccatattttctctcaatttgagttctaagatgaaattcagccctatgatatgggaaggattgtaattaagtgataaataaagggattttttcccccaaagggtatttaatgtgtggaaacctctttaagggtctatagaattacctgaggaagtattagaaaataagaagtgggagttaagtatttttttctgtcatcacaggaagcctgaaatctttttttttttttaagtacttatccaaatcactattcttttaaaatcttacaaaatcttacttctggttcatattgccagcatgttgaaatgtaaaaagagcattaggttggaaatcagaggatttttaatgatgttgaggaagttattatttataattttctgttcgttttcatttggaatttgtaaataatagcaagctgcctttatgggggtatttgtgaagattaaatcagatatgaaaatgaaattgctttgtaagtggtggagttatctactcatatgaagtattattgtactaagtgcatttacataaagcacttatatgtttgaagtgtgatgccttgatgtgataaatactttttgactgggaagtttgatgaagaaaaaaagtcaaatgggttattcatttgatttcttggtagagattggtggctttttccagactgaaggaaacctggctcctctcctgactgtgactttgcactctgttgcatcaaatgtcctgaaactgcagtgtacttctgtggttgagcttttaattttggttttgatttgttttaatctcacttctataagtgtaagaatggtcatcagtctagtttgacaataggaagtagctattgtttttaatattctcaaatggatctttaagttatttcatgaacaatgagactacattgaatgtgaaatctcaggtcctctgagaaatacatttcatgtttaaagcattagtctttgagccaaagaaataatggagtttagatttttccttctggggaagttatctctggctatgagaggatctgacccttgagattaacttcaagttaggttgatatatagaatatttacgataaagaaaaagtattttagaaacaagtacatattggctgggtgtggtggctcatgcctgtaatcccagcactttggaaggtcgaggagagtggatcacctgaggtcaggagtttgagaccagtctggcccaacatggtgaaaccccgtctctactaaaaatacaaaaattagcctgacttggtggtgtgtacctgtagtcccaattactcaggaggctgaggtgggaggatcgcttgagccaaggagtttgaggttacagtgagctgtgatggtgccactgtactcccgcctgggtgagtgacagagcgagatcctgtctctaaaaataaagctataataaagacattaacaaataaaattggagctatttatcaaaactttgaacttaatactaattaataaagtagctcaaggaaaaaagtaagtagtgtaagctaccattaggcaaattatgctgagaagtatagtaagtgctatagattcaggggaaaatgaaatctgtatgtcagaaacagtgatctaggaaaatttcatgggagaagacagctttaaatacagaaatcagaatgaaggaagaaaagcatttcaggaagtgaagacatggagttagggttgtccattttgtgttcagaagacagcaattaaattacttcagccatatgacgaggaaagctaatcagattagttgcaaattaggtgagaaccagatagtgggagtttttcagtgtcacactaaggagtgtgtctttattgtacagaatacttactcttaaagttgggcagactctgagtgggaagaaaaatattttagcactgggtataagtgtatgtaacctgcagtctctttcatctttgcctctatgtttatagggtagtagtggtttttttttttttttgagacggagtttcattcttacgcccaggctggagtgaaatggcgtggtctcggctcactgcaacctccacctcctggttcaagtgattctcctgcctcagcctcccgaatagctgggattacaggcatgggccaccatgcctggctaatttttgtatttttagtagagacggagtttcgccatattggccaggctagtcttgaactcctgacctcaggtgattcacccgcctcggcctcccaaaatgctgggattataggcatgagccactgcgcccagcagtagtgtgtttttagggagggacagggaaggttgaaaaatggcactgtagaaagccatcagtaccatagcaattaaaccagcgttgtagtggttcaggaatagaaaatagatcagtggaacagaatagagaacacagaaatggattaataaatgtgtgggactttatatgacaaaaggtggcatttcaaattatagaaaaagtgtggaatattcaatatttggtttaatttccacttcacaccaaaagaaaaaaaatccaaatgcatgaattaaacagctaaatgtaaaagccaaaaactgtggaagtattagtagaaaatacaggagaataattttatagtcttgggagcaggaaaaactttctaaaacaagatacaaaatctagatactataaaggacaccctgttttccagcctctacattgactgtataaaatttgaaacataataaagatactttaaaaatgtttgaggcatgctagaacattaattcatgacccaacttataaggaactacaaatcagtaatgacaatctaataggaaagtgaacatatgaatatagatgcttcacagaaaaggagatgaggatagccagtaaacacaagatgcacagccccactgaagtcttcctggaagtgcagattaaggaatttattattttgctcatgagatgagcggaaattaaagatggttgttactctgttggtgggattgtggagaaataggtactttcatatgttgttggcagcaactttgtactacctttgggagagcaatttagcaatatctataattttttttgtttgttttgttttgtttttgtttttttgagatggagtttcactggagtgtgcctggcctttttaatttatttttgagatggagtctggctctgtaacccaggccttagtgcagtggtgtgatctcggctcactgcaacccccacctcctgggctcaagcgattcttctgcctcagcctcccaagtagctgggattacaggcacgcaccatcatgcctggctaatttttctattttagtacaggtggggtttcaccatgttgaccaggctggtctcgaactcatgacctcgtgatccgcccacttcggcctcccaaagtgctgggattacaggtgtgactcaccacccctggcctttttttttttttttttttttttttgaggtagagtatcttgtccaggctggagtgcagtggtattatctcggctcagtgcaacctccacttcctgggttcaagcagcaattcttgtgcctcagcctcccgagtagctgggattaaaggctcctgccactatgtgggcctagctaatttttttgtatttttagtagagatggggttttgccacgggccaggctggtctcaagtgatcctcccaaagtgctgggattataggtgtgagccaccacgccctactaatttgttgtatttttagtagagatgggtcttcaccatgttgcccaggctggtctcaaactcgtgagttcaagcgatttgcctgccttggcctcccaaagtattggaattacagacgtgagccaacgggcccggcctgtggcttttcgtggagttcttagaacgtaagttttctgtgctgttgtgccttctactttctctaccttttg

SEQ ID NO: 9 - DNA, 50000 bp, Homo sapiens
  human DYXC1 chromosomal gene region, nucleotides 150001-200000
gaatgtcttcactttccagtcccttttacctagataatttgaattactctctgaaggctcaacctacttttcatgagttacagatatttgcttcttaaggatctttttttatcagtttcattttgatattttagatgtgaattcaatttggttgacagagtcttgctctgtcacccaggctggagtgcagtggcacaatcttggctcactttaatctctgcctcctggattcaagccattcttctgcctcagcctcccaagtagctgggactacaggtgcccaccaccatgcccggctagttttttttttttttttttttttgtatttttagtggagatgaggtttgcaccatgttggccaggctaatctggaactgtacccgacctgacagttgatctttttaatatttagcttagcttatctctaggtgtttcatccatagaactaaatctcagttgtgttcaggtacaaaaaagatggaaaactactcattcattcttcagaactgttttcaggacttcaaggaggccttccctaaccaccctatttaacattccagttcttgcaccatccccacccctactctgccctcacttttttctccatggcactgggcaccagctggctaactctgtcttcaaaaagtacttttctttgggggaggtttttcttttgtttttgttttctttctttcagtcctgtatcttcagcacttagaagagttcatggcacacagtaggtactcaataagcatatgttgaaagaatgaatgcagtttcatgttccggatgaggataggctcatttaaaatattttcatatgtttattagacaaaaagtagccttcaaaaaagttttcatgctgggcgcagtggctcacacctgtaatcccagcactttgggaggccaaggcaggcggatcatgaggtcaagagatcgagaccatcctggccaacatggtgaaaccgcatctctactaaaaatacaaaaattagctcggcatggtggtgcgcacctgtaatcccagttacttgggaggctgaggcaggagaatctcttgaacccggaaagcagaggttgcagtgagccgagatcgcgccactgctctccagcctggagacagagtgagactctgtctcaaaaaaaaaaaagttttcattagttgggatcttgtatcctcttccctttctcaagtgatgtgttttaaagcagacattttcatatcctttggcggaggtgaaagacccagaccttttgaaaagtattttacaaatacgttcttgaaagcactccagactgactcgcttatctttggtcacatgacagtacagctgcactttgaggcctttgcctatagcctattggttgccttacttcggaattgtagacgtgtgggagaataatgctcccattcttctccatcttatctagttctagaagatataatggtaatggaaacctcacctttttcatagtaaaatgtagctacccaggaatgatgaggggcaccatcatatcaggtaatattcattccagttatattttatgaggtaaaattgatactgttgttgtgttgttcttacagattttgacaggctccaccaaagtgtttttaaatgacttatttagcctctaactctcccctgaattcagtctccagacgcctactttatgtttcctcttggatgtgataataactatctcagatttaaaatgccccaaaccaatttgctggtttccttttcttcttctttttttttcttgagatggagtcttgttctgtctcccaggctggagtgccgtggcatagtctcggctcactgcaacctccacctcccgagttcaggcgattctcctgcctcagcctcccgagtagctgggactacaggcatccaccagaccacctggccaatttttgtatttttaatagagacagggtttcaccatgttggtcaggctggtcatgaactccttacgtcaagtgatctgcccacctcggcctcccaaagtgctgggattacaggtgtgagccaccatgcccggcccaatttactgattttcttctcataacctgctctttttgaagtctttctgcctcagtagctattccattctagtactcggtgaggccaggagtcatcctttataccccatattcaatacatcaggaagtcctgttggctgctgtacctttgaaatccgtgtataatctgtccatttctcactgtcttcactgccaccattctagtccaaaccaccatcatgtctcacctggattatcatagtagccatgcaaatgaacttcttgcttctgcctttgctcccccacaatgctgtcccagtaggtagccagagtgatcttttattccttttctcaaaacgcttctgtgacttttagttctcagagtgtaaagctaaaacgctttttaaaaggagtcttaggatctgtaagccctactcagtctgtccccttgccaccactctgtttattatcttgtgggtttttttttttgtttgtttttttttgagacggagtctcgctctgtcgcccaggctggagtgcagtggcgccatctcggctcactgcaagctccgcctcccgggttcacgccattctactgcctcagcgtccgaagtagctggaactacaggcgcccgctaccacgcccggctaatcgcccggctaattttttgtgtttttttgtagagacagagtttcactgtgttagccaggatggtctcgatctcctgacctcgtgatccgcccacctcggcctggagtgctgggattacaggcgtgagccaccgcgcccggcttatcttgtgtttaatcttttcctcctctctcctgctaggcgatccacacaggcctccttgctgtttctcagaggtagagctctgatctcagctttatagctactatttcttaaatgctcttcctccagttaactgtatataatggtgcctcactcattcaaatctttgctcacatatacttcagcaatgaagcttgccctgaccatcgtttttagagttaagcttctctatcccagcataaatctatcccagaataaatctcctcaaatattttacagggtttgactgtttttgtcgacataagtctaaggtttttgacctgaatgtgtggtgactcttgcttgtaatggctgtattgtttttggctactgcaattctgtgagttagcagaaataggagcagcaggaagatgatttcagtattgtagctgttgtgtgttaaacacttgagaaacctgcaaggggacatcaaaagcaattggaaatatatgactagagatcgagttaaagaaactactctgaggaataaaaggaagcctgagcctaaacagagcttcaagtttattgtcttcacaaagtattcttctttttataaccgtatgatttcttagcctttgtaactgtcctgtaaattctgcttatttctaaattactattagtaaagctcttaaaatggggagggtttatacatttaccaatggtcagattaatgggagaggaagctcatggatttctccagaattttgagttcttttttttttttttttttggaggtggagtcttgctctgttgcccaggctggagtgcagtggtgcgatcttggctcactgcaacctccacctcctggtttcaagcaattctcctgactcaggctcctgagtagctgagactacaggcgcacgccagcatgcccgactaatttttgtatttttagtagagatggggtttcactatgttggccaggttggtctttaactcctgacctcctgatctgcctgccttggcctcccaaagtgctgggattacaggtgtgagccaccgcacccagcctaattttgagttctgtaaagaacaattaactgcactatcacccagacgtggttacctttcttgtttattgattgcttaatatgtatttagcattcattcaatatgtgttgggaatacattataggctggtaaacaagcaattacagcacaatattctatattaagtgccagatgcagaaaataataagagtcttagggatggggtcaagggatggttctcaaaggaaatgaaccgttagctgagacaagcagaggagtttcggtggaggtaaaggggatgggcaactgatcagaatatgtaaagccctggagtaaagacaaagccaagcattttgaggggaactgagaaggttgggcaaagcgggaatgaattaaaagagatgagagctagagataaaatacttgatattcaaggctttggggcattatcttgaagaaaatcttaacattttaaaggaattacataatgagattatcatttgaagatctctagctataatggggaggttcaaactgccagggggctttcatattcactgtgtctaggatagggcttaaaaagttacttttttttttttcctttgattgtagtacacgcccaacattttgagaacagttgaattaatagggttctttagtaagcctgttagttttgcttatccaaattgtcatggtggtgttagggccagcgggaaatctttttctcttttgggctggatggtgttaccaaaacaccaggggtttggtctaggtcctgctgcttgccgcacagaaagccaatcacagagacgggtattgccagggaagaaggctttaattgggtgctgcagccaaggacatgggagatcagtctcaaatccatctcagtctcaaatccatctctgactgactaaaattagaggtctatatagcagagaagaaatgtaaccatgtgtgggcaaacaggaattagagaggggtgaggaagagaagttggtcaacaggcagcaggtggtcagttaagcaatcatgcaggctctggcttctcattgtccagttgcagtgatctgttaagtaagtttcagcttcttaatactatctaggaggcctgatggtttgtttcctaagaaaggagcttagataacacaaatagaactttctcaagttttaagactgtgaggatcaatttctgtatttattcagagaaaccataaagatcagttctgtgggataattggctggtttcaatgttatctttccttgaaggaagtaaacaaagcattaaactggggcctgattaaagtgggcttattgatttgagaaattcaagtatggtaagttgtcccaagtcagtaatgtattttacttttttttgtaatctgttagaagatgggagcaggcaaagggaaggagggaagctttcacacagtggagaaagaatgctttttaggcttcagccagtttttcttatcacaaagccctcacctacttcttgtttcatatagaattcttccagttacccagtcacttaaaccagaaacctgtgagctatcctacactttctcccttatatagttggttattcagtcctttaataaaataatctttaggctgtgtacagtggctcacacctgtaatcccagcactttgggaggccgaggtgggtggatcacttgagcccagaagtttgagaccagcctaccaaactagcaagaccccgactcttaaaaagaaattagctgggcagggtggcacatacctgtatagtcctagctacttgggaggctgagatgggagcatctacttgaacctgggagtttgaggctgtgttactggtggagggtgtccaggttcttggcatcttgaacaaagaactggacaaaacacacagacaaagcaaggaagggatgaagtcagagatttattgaaaatgaaagtacactccacagtgtggtggcgggccctagcgtaggggctcaagggcaccgttatagaatttgtgggggtttcaataccctctagaggcttccattggttacttggtgtataccctatgtgaatggagaggtgaagtaaagttacaaagtcatttacttggtgtatgccctatggagaggatatttcctgtcatagctgaagtgtgaatcggcctaatgttccctgcctccagaccctattttcccatctcagcaagccgtgttcatactactgtactccagcctgagtgacagagtgagaccttgcatccaaataatgataaaaataataaaataacctttcatgtcttctcagatctcttcaaaacccttcccgctatcttctaaaccctttccctcatcaacactggttaagttcagatgctcatatttttcatggaatattatgttgcttccttgcatcccacaggtctagggttgttgagttgcttccctatcttgaaaaaaaacatttttttgagacagggtcttgctctgttacccaggctggagtgcagtcatgcagtcatagctcactgcaactttgaactcctgagctcaaatgatcctcctgcctcggcttcccaagtagctaggacaacaggtgtgggccaccatgtcctgctgattaaaaaattttttttggtagagatggggagtctcactatgttgcccagcctggtcttgaacgcctggcctcaagtgatcctcccacctcagcctccgaaagtgctgttacaggtgcaagccgccactcttggccctgccatcttaatatatgctatttaatttaattaattaatttatttattttgagacggagtttccctcttgttgcccaggctggagtgcaatggcgccatcttggctcaccgcaacatccgcctcctgggttcaagcaattctcctgcctcagcctctcgagtagctgggattacaggtgcttgccaccatgcccggctaattttttgtatttttagtagagacagggtttcatcatctttgccaggctggtgttgaactcctgcccttatgatctacccgccttggcctcccaaagtgctgggattataggcgtgagccactgcacctggccaaatctgaccagatacaaccgcttatgcctgtcatcctagtactttgagaggccaagaaaggtggatcacttgagctcaggagtgtgaaaccaacctgggcaacatggcaaaaccctgtctttacaaaaaatacaaatattaggcatggtggcgcacacctgtagttccagctactcaggaggctgaggtgggagggtagcttgagcctgggaggtcaaggctgcaatgaactgtgttcatgtcactgccctccagcctgggtgacaaagcgaaatcctgtctcaaaaattaaaaaaaaaatcatggccaggtgcggtggcttcatgcttgtaatcccagcactttgggaggctgagttgggtggatcatgatgtcaggagatggagaatctcctggcctttagtagagatggtgaaaccccgtctctactaaaagtacaaaaattagctgggcatggtggcgcgtgcctgtaatccccgctactcgggaggctgaggcaggagaatcgcttgaaccagggagtcagaggttgtggtgagccgagatgacgccactgcactccagcctggtgacagagtgagactccgtctcaaaaaaaaaaaaaaaaaaaatcaaatctgatcttttaaactatatttgtactagattgtcttttttattttttatttttttaaagaactttcatattcttttacatttttctgcctttgtttttcctgtgtgatgctttttcctttttcactgggacatctttccttgtcatcagatcttttgattttttttttttttttttaggaatcagaaatcgttttgtttgttttttgcaatgttgcccaggctgatcttgaactcttggacttgagcggtccttccgtcttggcctctccgtgttggggttacagacgtgagctaccacacctagcccccccccccctttttttttttttttaaagagtcaaggtttcactgtattacccagtctggagtgcaatggcagttcacaggcatgatcattatggctcttaacagccttgaactcctggcctcaagggatcttccttcctcagtctcccaggtagctgggattaataggcactctccactgcttccggctccagaaatcttcattttaatgtagtatctcaatttttaaattttgacaactgttaatttactttaaaaaatgctatcaaatttgcagcctcatttatagagatttccttgatcattctcctttccccctgcaaatttaattgctcagtctttgtgtatttctgtcattgcataattttcaagctattataaacatgattctcatacaaatagacattgatacatatcataaacttttttgaaagtcctgaaaaatagtggaatgagaaattgggatttatatagtcagtttaggccaggtgcagtggctcacgcctgtaatcccagcgctttgggaagctgaggagggcggattgcttgaggccaggccttccagaccagcctggccaacatggccaaacgttgtctctacaaaaaatacaaaaaattagctgggcatagtggcacatgcccatctacttatggggctgaggcaggaggatcgattgagcttgggaggtcagggttgtggtgagccatgattacgccattgcactcaagcctgtatgacagaacgggaccctgtcacactcaaaataaagaaaagaaatatagtcagtttaataaagaatgctgaaataaaattgctaagatataaacgtggttaatatcctacagggtatttaaactgtagctttttggataacctctactgactggacagaaaaaatttattttaccgtagtagttttctgtaagtttacttttaaatttgtagagttataaactgcctgggccttaattgaaattagaaataaaacaattagatgacctcaggtgagcctattacaaaatgctcttgtcataacattaaaagtcatacaattgataccttatttcaggttttgaataattcatttgaaacatgttaatttttatactattagcgaccaacctttttgtatcctctctttttgaaatattaatgtacaggagttttcagaataattaacagattatatagatctgacttgaggctaggagtttgagaccagcctggccaacattcgcaacgagtgaaacctcgtctctactaaaaataccaaaaaacaaaacaaaacccccaaaaacagattatatagatctgtatttttgccctgttctcatttgagcactagttccatattgtcttcagtagagttctattagaacttggatctaatgtgttcacagtatatgtccaataacagaagcataagacacatgtagaataaatcagtaggagtactatttgctttactaagttgggttttgaagtttaaaggagtttcagatacaaaaagaaaaaaccaggtggagtgaccattgtatgctggcttagggcttgagtccaggttgttgccagtgaccagatgtttgtaaccaacctctggtgtcttcccccctgccccaactttaacacaactgaagtattgctgacacagttattttcataaaaacagtttgattgccgggcgcagtggttcacgcctgtaatcccagcactttgggaggctgaggcaggcggatcacgaggtcaggagttcgcgaccagcctggccaacatagtgaaaccccgtctctactaaaaatacaaaaattagtggggcgtggtggcacgcgcctgtagtcccagctactcgggaggctgaggcaggagaatcacttgaacccgggaggcggaggttgcagcaagtcgagatcgtgccaccgcactccaaccggtcgacagctcgagactccgtctcaaaacagtttcatcatgtttcttctatgtttgaaaatgtaaattacttcatgttgccaacatacagacttcattttcttggcataggagcttttcagtttccctgtgcttctcagtttctgcagtcatgtttttcacaccttactgttcattgaaaatagagtcctttactgttgctgcctgctccccattcacgattatccttttccttcacctctgtctggccaaatgctacattttcttttagacataactttctccttcattccaacaatataactgttgagactgttctagtccaggagggtctccttatcttagaagtttgcttctatctgttcatacaggtatgcagtcctgtcatgttctgtatggtaaatctatgggtggcttgtctctgtttcacctggaagacagtaaaccacttttcaaacttagtgtcacatatagtgcttcctatagaattaaaaaaaatgtgtctcatgagtcaatatgtttttgtatttgtagacctgaaagatgtctgaaaattccagtgacagtgattcatcttgtggttggactgtcatcagtcatgaggtatgatatttaatactttttataaggtaatttgtaatattaatgttaccagtgatgtaaactgtgttctttgaagatcagtctttttctacctttaagcaaaaagttccaatggacattttccctctcatattaaacttcactgctgaaaacgcttcatgagccagcatcttctacattattgtacatagctttgagacattaggcaaatatttattgaaggtttgttattgttcaggccctgtgctttactttcattttcatataattttgtctttttttttttttttttttttgagacagagtctcactctgttgcccaggctggagtgcagtggcgtgatctcagctcactgcaacctccgccccactggactgaaatgattctcctgcctcagcctcctgagtagctggaattacaggtgcctgctaccatgcccagctaatttttgtattttcagtagagacgagtttcaccatcttggccaggctgatcgtaaactcctgacctcgtgatccatctgcctcggcttcccaacatgctgggcttacagatgtgagccactgtgcctggccaattttgtctttttttttttttaaagggacaggatcttgctctgttgcccaggctagagtgcagtggctgttcacatgtgcaatcatagtaccatgtaacctttaactccttgggctcaagtgatcctcccaagtagctgggactataggcatatgccaccataccctgttcttaaataattttgaatatttcactgtgatacatactgttttcattcttattttatagttgagaaaactaaagcttagagaggttaagtaatttggtcaagattatgcagctagaaaatagatcctggatttgaacagatcagaaaggagaatggagaacatgtgttccatttacgttatagcagaacttcacagggaaaaggccagaaccctatcacaaaatgaattttataatagaagattgatatacatacaacaagtatacataacttgtacattttgtttattttttcataccagtaatatatgttcactgtagaaaaatggcagtatattgataaacagaaatatacttactccaggttttactccaggttttaatggacatctataattttcccatttgtttttagggtattgtaagcctcacttgttttctttttttcttttttgagatggagttttgctcttgttgtccaggctgtcgctcttgttgtcttgtacagtgacgcaatctcggctcacagcaaccttcgcctcctgggttcaagccattctcctgcctcagcctctggagtagctgggattacaggcatgtgccaccacgcccgactaattttgtatttttagtagagacggggtttctccatgttggtcaggctggtctcgaactcccgacgccaggtgatccgcccgcctcggcctcccaaagtactgggattacaggcgtgagccaccgtgcccggccacttgtttttttaataaaaattataggccgggcgtggtgctcacgcctgtaattccagcactttgggaggccgaggtcggggcagatcactgaggtcgggagttcgacaccagcctgaccaacatggaagaaacccggtctctactaaaaatacaaaattagccaggcgtggtcgcacatgcctgtaatcccagctatttgggaggttgaggcaggacaatcgcttgaacctgggaggcggaggttgcggtgagcagagttcgtgccattgcactccagcctgggcaacaagagtgaaactgtctcaaaaaaagaaaaaaaaaattaaaaaatttttgttttttaaactattagttaaggttcaggtagttatgtttagaaaacattaacagaaaaaaacaagattgccttttctgcattaaaatttcaaaatttaaaagcatctatcatttgagattgagatgattcttctttttaaaatcttcagtgacttttctatttttagcttgttttcttcaaaggattagatcaactcccatagcaacttctccttcaaggataattaacaatataggagcaaggcagctatgggagtcttacctgttagtctgcagggtggagtatgacagcagacctctgtctggcactcagtcaagtgtgtcattagggtgttctgtactggatgttatgtcccagattgaatcatatttgtacttttatctctcttcttctatatctgctttccaggttctactctttcttgctaatttctcttacatgtgtctccttttcctctcccattactgccattctcatttgggccagaattggtttccccagcaattcctaatacataaccttgctttagcctctaccattctcctgccactatagacaacctcggagtcttgcattccgaattctcattagggctcaacttaaacgctacttttttctttacttttttttttttgagatggagtcttgctctgtcgcccaggctggagtgcagtggcaggatctctgctcactgcaagctccgccccatcggttcacgccattctcctgcctcagcctctggagtagctgggactacaggcgcccgccaccacgctcggctattttttttttgtatttttagtagagatggggtttcaccgtgttagccaggatggtctcgatctcctgacctcgtgatccgcccgcctcaggctcccaaggtgctgggattacaggcgtgagccactgccccggccctacttttttcatgaattcttttaatttttgctgtcttgagttatgtatacgcaagagaatgcagacctttcgattccatgtgtcagcagactatagtggggacatagggaaagacggaatcaccttagtggttaactttgttttacaattaagacacaaagaaaaattgtatgatccgctattattaatgaaaagacagtctgacatcaaagtaggaaagataacctcagaataaaaagatagtctaaaattgtgtgaggagagtattcattaaccctataggtttatttaatccatgtcattaaagctttagccatttttagtttatagtgccataataagtagttatttgcatatgggtgtttctctgaatatgttgtaaaattgaattttggatttcattttttttttttttttttgagacggaattttgctttttttgtgatccaccggcctcagcctcccaaagtgctgggattacaggcgtgagccaccgtgcccagccagatttgaattttttatatcccacagcatctagcgaagtgccttgtctagagtagttccataaatgttaagtagaaacaaattattatagaaataaccaaatttagacccttattctctggaattcaaatttattgcttgagctcataaaatagagcattccacttttgggctgacctgacttagaatttaaacctgagactccattgtgccattttcctgccataggcctttgtggactaaattgaacaaaagcttgtctaattagatactgttcctttggagtatggcagcatctgagacttttaataatggaagaagcatttacatgtagtcaaatctagctttgaggagctaataggcaaataactgaaagattatcacttttccttaggggtcagatatagaaatgttgaattctgtgacccccactgacagctgtgagcccgccccagaatgttcatctttagagcaagaggagcttcaagcattgcagatagagcaaggaggtaagttgttcacaaattctaattttttttttaatatgatatatgagtgatattatgagtcttgccttctttccaggtgggtggcctttctggctaatctaaggggtcgttggaggattagaacttcagtgtaggttgggcacagtagctcacacctgtaatcccagcattctgggaggctgaggtgggcggatcatttgaggtcaggagttcaagagcagtctggccaacatggtgaaaccctgtctctactaaaaatacaaaaaattagccgggtgtattggcaggtgcctgtaatcccagctactcagaaggctgaggcaggagaatcgcttgaacctgggaggcggagtttgcagtgagccgagattgtgccattgcactccagcctgggcaacagagtgagactccatttaaaacaaacaaacaaagaaaaacttcagtgtggatttggtgaaggcccacagaacccagagtcatcagcctgccccaggggtttatttgcagctgtagtatatttttgggttttcagatacactttgttgctacagaggtaaccagtgacaaaaaaagtgtttcatggtttaatctgaatttgtttggtagtttctatttccttgtcatttgtttggtagtttgtatttacttgtcattattaaagttaagaaaaaaattattcattgatcacaagagatttactaacctgttttccagttattatcatgttgatggagttacagacatgcctctcattttattctgctttgctttattgtacttcgcagatactacattttttacaaattgaaagtttgtggcaaccctgtgttgagcaagtctattaatgccatttttccgatagcatatgcccacttcatgtctctgtgtctcatttttataattataatatttcatttatttttattttattttattgaattgaattggcggagtttcgctcttgtttcccatgctggagtgcaatggtatgatcttggctcactgcagcctctgcctcttgggttcaagttatcctcctgcctcagcctctcagctgggattacaggcatgcgccaccgtgcccagctaattttgtgtttttaatagagacggagtatcatcatgttggttagcctggtctcgaactcctgacctcaggtgatccacctgtgtcaacctcccaaagtgctgggattacaggcgtgagccactgcgcctggcctgtaattgtcatatttcaaactttttcattattattacatcagttatggtgatctgtgatcagagatctttgattttactattgtaattcctttgggacaccatgaaccgtgcctatgaaagacagtgaacttaataaatgtgtgtgttctgactgctctactgacttgccattcccccatctctctccctctcctttggcctccctgttcctggagacaacaacaatattgaaatcaggccaattaataaccctacagtggcctccaggtgttcaggtgaaaggaagagttgtgtgtctctcacttcaaatcaaaagctaaaaatgattaagcttatttgaggaaggcatgtcaaaagctaagtctcttgggccagatagttagccaagttgtgaatgcaaaggaaaagttcttgaaggaaattaaaagtccttcttagtgaacacatgaatgataaggaagccatatagccttattgctgacatggagaaaattttagtggtcttggtagaagattgaaccagccacaaaattcccttaagccatagcctaatccagaacaagtccttcctaacttgcttcaagtctatgaaggctaagagtggttaagaagctgtagaaagaaaggcggaagctagctgaggttggttcatgaggtttaaggaaagaagctatgtccgtaacataaaagtgcaaggtgaagtagcaagtgctgatgtagaaggcgcaccaagttatccaggagatctaactgagataatggatgaaggtggctaccccaaccagcagattttcagtatagatgacacaactttctattggaagaagatgccatctaggactttcctaactacagagaagtcaatatctgcttctaggcttcaaagaacaagctcactttcttgttagaggcaaatgtagctggtgactttaagttgaagccagtgtttacttaccatgccttgagaaatcctaggacccttaaaaaatatgctacatctactctgcctgtgctctgtaaatggaacaacaaagcctaggtgacagccatgtgtttacaacatggtttactgaatattttaagctcagtgttgagacctgctgctcagaacaaaaagattcctttcaaaatactagacattgtatagcaatacctggtcaatcaccgaagggctctgatggagatgtacaaggagattcatgttactttgtttcctgctaacacaacatccagtctgtagtccatggatcaaagagtagttttgactttcaggttgtattatttaataaatacattttgtaaggctgtagctgctatagatagtgattgttctgatggatctgggcaatgtgtgtgaattgaaaatcttcaggaaaggattcaccattctagatgcctttaagaacaattgtgatttgtgggaaaaggtcaaaatatcaacattaacaggagtttggaagaagttgatttcaaccttcatgactttgagtggttcaagactttaatggaggaaggaactgcagatgtggtagaagtagcaagagaattagaattagaagtggatcctggtcgggtgcggtggctcatgcctataatcccagcactttgggaggctgaggtgggcggatcatgaggtcaggagatcgagaccatcctggctcacacggtgaaatcccatctctactaaaaatacaaacaattagctgggcgtggtggcaggcgcctgtagtcccagctactcgggaggctgaggcaggagaatggcgtgaacctgggaggcggagcttgcagtgagccaagatcgcgccactgcactctagcctgggcaacagagcgagactctgtctcaaaaaaaaaaaaaaaagaagtggatcctgaaaatgtgatccaattgttgtaatctcatgataaacttgaacagataaggagttgcttcttatggatgagcaaagaaagtagtttcttgagatgcaatctactcttggtgaagatgctgtgaatattattgaaatgacaacagaggatttagaatattacacaaacttagttaataaagcagtggcagggtttgagaagattgactccaattttgaaagttgttctactgtgggcaaagtgctatcaaacagcattgcatgctacagagaaatctttcatgaaaggagagtcaatccatgtgggaaacttcagtgttgtcatacttcaagaaattgccccagctactccacccttcagcagctaccacagtggtcagtcagcagccatcatctttgaagtaagactctccaccagcaaaaatattacaattcactgaaggctcagatgatcactagcactttttagcaataatgtatttttaaattaagatatgtacagttttttagacatgatgttattgcacacttaatagattacagcatagtgtaaacctaacttttacatggacagggaaaccaaaaaattcatgtgacttgctttattacattggtttggaactgaacttgcagatatctgagatatgcctgtactctggttccaatctggtaaaatggaatttgactactgtattgcatctgtattatgtatacttcacatgggatttgatgcacacattatttatatagagaaataggactcctgaacaattactgcatacacttatgttcgtatttaaattacttcattaactttttaaaaaaattacagtaaaatgcatacagaacatttcccctcaggagaaaaaagtggatgtgtcaaggccagaaatagaagagattatagctgttaatttttttaagcctacacgttgattattactactctcatttgaaaagtgattctcatcttttttgtgtcagagatccttttgagaatctgaaagctatggacagtttcttccaagaaatgcatttacttgcatatgttaaagaaaattttcttttttttctttttctttatgagacagagtctcgcactgttgcccaggctggagtgcagtggcacaatgatctcagctcactgtaacctccgcctccctggttcaagtctcagcctcctgtgtagcttggattacaggtgcctgccactatgcccagctattttttttttattttttatttttagtagagacgggggtttcaccatgttggccactctggtctcgaactcctgacctcctgatcagtctgcctcggcctcccaaagtgctgtgattacaagcatgagccactgtgcccggcctaatttttttgttttttgagatggtgtcttgctctgttgcccaggctggagtgcagtggcgggatctcggctcgctgcaagctccgcctcccgggttcacgccattctcctgcctcagcctcccaagtagctgggactacaggtgcccaccaccacgctcggctaattttttgtatttttagtagagacggggtttcactgtgttagccaggatggtcttgatctcctgacctcgtggtctgtccgccttggcctcccaaagtgctgagattacaggcgtgagccgccgcgcccggccattttaatagcgacgggatttcactatgttggtcaggctggtcttgaactcctgacctcgtggtctgcctgcctcagccgctcaaagtgctgggattacaggcatgagtcaccgcgcctggccccagcaccccggccgagtactgtggcggaatctcacctctaaattaattaattttctgttagagggtatataatgctgaaaaatatttttttttccctgttctttgcaggacttaaattacataagaaatgcatgatcgaccgggcgcggtagctcatgcctataatcccagcactttgggtggccgaggcgggcggcttacggggtcaggagatcaagaccatcctggctaacacggtgaaaccccgtctgtactaaaaatacaaaaaattagccgggcgtggtggtgggcgcctgtagtcccagctacttgggagcctgaggcaggaggatggcgtgaacccgggaggcggagcttgcagtgagccgagacggcgccactgcactccagcctgggcgacagagggggactccgtctcaaaacaaaacaaaacaaaaaaaaaacaaaaagaaatgcatgttcatgatagggaaaaaaaaatagtaagcaaaagaaatttaaaatggtctataattcttttatctagaaataattactgttgatatttggtgtactttgctcataaatttatatgttatattatttttaacttttcttaatattttatgctcttttctttttttttttttttaagacagagtttcactcttgttgcccaagctgtagtgcaatggcatgatctcggctcactgcaaccttcgcctcctgggttcaagcaattctcctgcctcagcctctggagtagctgggattacaggcgcgcaccaccacacctggctaattttttgtatttttagtagaaacggggtttcaccgtgttagccaggctggtctcaaactcctgacctcaggtgattcacctgcattggcctcccaaactgctgggattacaggtttgagccactgtgcccggcctttatgctctttttcattaaaaatatattgtgaagtcatcattctgtttgtacacattttctttgaggaggttcttataaaatatatcttctgtagtgacatttagtttttctgtgttttgagttctttcttttttaaaaaaaattattggtagaactggtctcgctatgttgcctaggctggttttgaactcctggctttaagtgattctcacacattggcctaccaaagtcctgggattacaagcatgagccactgtgcctggactgccctctctctgacttttttttgagacatagtcacactctcttgtccaggctggagtgcagtggcatgatcatggctcactgcagcctcaactcctaggctcaagtaatcctcctgcctcagctttctgagtagctagtactataggcacataccaccatgcccaaataattttttaatttttatttttgtagagacagggtcttgctgtgttgcccaggctggtcttgaacttgtagcgtcaagcagtcctccagccttggcctcccaaaagtgctagggtgacaggtatgagtcactgcaccccgcctgagtccttgttttttatagtttatgttttcttataaggcatttagacacacacacacacacacacacacacacacacgtacatatacatacatactttcataaatcatagtttgaagccacggtttttattcttacggtaccaggcattgacagccattagaatggtctttagaaacttcgaataaacttttaagaattcaggaaatgtatcactaggtatctaatgatagattttttcatattaggtattaatgaacatactggatcaagtaatttggtaatgtaaggtagaggttttatttacttagtcaaggtagctttgagttcttaataaatcttgtggctgggcacgatggctcatacctgtaatcccagcactttgggaggccaaggtgggcggatcgcttgaagtcaggagttcgagaccagcctgggcaacatggtaaaaccctatctctagtagaaatacaaaaattagccgtggcgtatgtctgtaatcccagttgcttaggaggctgaggcaggagaatcacttgaacccaggaggcagaggttgcagcgagccaagatagtgccactgcactccagcctgggcaacagagcaagactccatctccaaaaaaaaaaaaaatcttaaatgattgcattttcttatccaaaattatcacctgacagtttcaaataaatgaagttcattagcaacagtggagtgacttctataattgtatgtgacatttcattaaatctatcctaacatattaggaagagatttttatctacccagattatatgttggctgcagaaagtcaacagagagttttaaattaagaaataattatattgatctttgctttatatcagaaagcagccaaaatggcacagtgcttatggaagaaactgcttatccagctttggaggaaaccagctcaacaattgaggtaaacttgtattttacactgtggatatatgttaaatagtatccttagaatactatttaaagcctctaattctatttatgcattctattcctattttgaattataagcaattttgacttctaagttagatgtcttagaggacatctaagcaaagtattttattggtattgtgttaaagatctatcttttcacctctaatataattctttttggaagtagattgcattcttgaattatattgaaaaattaaactgttaggtttcatgtctaaaatggtggttcattttaaaatttggtaacaacctgagacttatgctttggggttttacttgacttctgtataaaactagatagctagatgatcattttacatggtgttttagtctgtttgtactgatctgacaaaatacctgatactgggtgatttataacaaacagaaatttatttctcacagttatagaggctgggaagtttaagatcaaagtgtggacaagttcagtgtttgctgatgacatgctgtcttgtttccaaatggcattttgttgctacatctttaagaggagatgggcaaaaaaaggctggatgctgcatgaagcctgttttataaagaccttaatcatattcctaggggaggatcccttataactcagtcacctcctaaaggtcccacttcttagtactatcacattggcgattacattccagcgtagaaatttttggggacaccttcagatacattcagtacaagatacagctgttgaagtttattacgtacataacctaaaaagttcagtgagtcttttttgggcttccacactacagagttgtgctcgaatagaaccatggaactcgtgagagaagtgagctcttttttctgtggttattgagggggcaaagatgggcttctcagcttctgcagcccactaggagaatgcagtggtttgatctaattctgagagttgtcaagggatctgatggtgttctcaagctatatgatgagcctgctattgcataattctttactaatagtaggaatcatatcttttattttaactcagatgactataatacttactactgttgtattctaagaaaatgtaagacccttaaatgattgttgaacttaccatctctaaaattatgattcttcaggcagaggaacaaaagatacccgaagacagtatctatattggaactgccagtgatgattctgatattgttacccttgagccacctaagttagaagaaattggaaatcaagaagttgtcattgttgaagaagcacagagttcagaagactttaacatgggctcttcctctagcagccagtatactttctgtcagccagaaactggtaagaatacactgataagagacttaaagacatgtacgaccacattttatataaaggcttgttttctacccctaataatttagtctctagttttgaatatgtttgggtaaaactcaagacaagactatttttgtgtgtgaaatctattaatacctagtgtttcgctagactgttctggagcatagcaatcttttactgtaacagtaaccatcttttgccattaaatagaagacaaaattgcttcagtgagatttgttaaaatcacaaatgctccaaaattcttttggtgctttttagatctttaagagtgggagattggggatgttaatatttttgcttgcagtagtgtacattttgtaatatagaaatagtgaaaaaaattgttattaaagtctcttcagatatattcaattattaatcatgcaacaaacatttattgagtacctaccatatgtgaaacagagttcaaggtgctatcaatacagctagtcttccaaagctcgtgaccttcagatcttttgttttagattctgttatgaaaatgtcactagtgtaagccacatttttaatgcaatcaatgcccgttgtacagacagactcattcagagattaaagtgtgagttgcttgatttctttctgctatgcttcagctcagttttagcagtatgtataacatcttttaatgtgatgtaagtatgtaggtataaagttcctggatatatcagttattaacccacaattataactaagcgtgagttatggcagagacaacttgaaacactgctagtgattctaaaaatttcctaatttgacgaagaaacaggaaacaggtagtatctattttagataatttttttagattttaccattctttaatatttacatttgccaaccagaatcagacaatagaatttctgtcattccagtttaagctaataaaatgtcaagtagaggttcttggctgggcatagtggctcatgccagtaattccagcactttgtaaagctgaggcaggtggattacctgaacccaggatttcgagaccagcctgggcaacatagcgagacctcatctctacaaataataaaaagccaggcatgatggcgtgcgcatgtggtcccagccacttgggaggctgaggtgggaggattgcttgagcctgggaggttgaggctgtagtgagctgagattgcgccactgcacccagattgccaacctgggtgacagagcaggatcctgtctcaaaaaaaaaaaaaaaaagtagagattcttggctatctcatttaatgagaagttattttcagttagtatatcagtggtttgcctataaacatgaacgtgaaaacactttttccagctatttgaatattcatgtgacattcttgatgttcagaggaaaacttcaggctttgttagaatagcattcatagtatctttatttatggaggggtattgtaattgattttagaatccttgttagtcatcaaattttattattatttttttaaactccaggatttgtgttgtattcaggtagtttccccacccccgcttcctttcctgcctctggtttaattagagaatattcatggcccagaaaagtcaaattgaagccagagtattatagaatggtcagatatataagaaactgacatagacgtaaaaggaactacatactgttgctgggcgtggtggctcacgcctgtaatcccagcaccatctcaaaaaaagaaagaaattacatattaacttttttcaggaagttgtatagttctaagaccattcttctaatatttatcttctttctgaattctgacttcttgagatttttgtggactaagaggaatatttgattaactgtatttttacccattgttattcctcctaaagatattgtttcaataaaccaaatgtttaaagacgaaattttggactatactgaaaacactgcatataaaccagttgccaaaagtttttgttttggggccgggcactttggctcatgcctgtaatccctgcactttgggagactgaggcaggcagattacctgaggtaaggagtttgagaccagcctgaccaacacggcaaaaacccttctctactgaaaatacaaaatttagccaggtgcctgtaatcccagctacttgggattagctgcctgtggtagcgcaggcctgtaatcccagctactctggaggctgaggcaggagaatctcttgaacccaggaggcggaggttgcagtgagccaagattgcgccactgcactccagcctgggcaatagagggagaccccgtctcaaaaaaataaaaaagatggtttttgtttttcccttttagtttgttaattttgtgctagccattgcttaattttgttttttcctttccctttcttcatccttaaggatttcaacttcaattttttcttctcctgacttatattcttgtggcagtgtacgttttgttgggttaagaacactgcctgcttcctctttatgaactctcttccctgacaaaaagcaaagttggtagtgtagagaagtttgtattgtgtgtttttaagaaggcatggttattacgttttaattttgtacggataacaatgttaagtgataattaagtatttttaattcttttttttctttttagtctttataatatagctttatgcctctttttttcctgtttattcacaaaagaaaggtggtgggagaagctgtggaagattcctgagtgcatatggggatgggatgatcagctgaaacaccatgttcctagccaactcgctttccaaggtggtgtgactaaatctgaccagcttctctagagctgagtgtgcctagtcacatttactatttaccgagtgctttaaagatacctgttgtcatccgcaatactctaaacaggtgttcagaatctcttgtaaaggagctggccaagcttattgcatgcctttgcattgcttgcatctatgggccaaattaagcttttttaaaagaagttaaaaatcaaaatatttcagctattatatgtatgggttaaaaaagttggaagcattagataaggacatgaatatattaatgtgggttaggctgttttgttagaatatataatgaaggtaatgttaatgtagaattgaacaattatttaaataagttttacaaatggtttgtttttacatttacctatcttaattttatgtcagaaagcctgacttaaatactctaaccatgatcatgaatgcctttgtattatgtaatgctttgtttaagtcttcctttctttctctttgtccaagaaatataatctcacacctgaggcgacggcatctttgaaaaactagtgatcaagaataaccagataaacatgatgggcataagggtcatgaccaaaaagatctctcagtggcatctgtatcactaaatattaggaatattcattcatttcagtagtgccttctgtctttgtgtttattgttgtaagtgtgaaagtgaactgaatactccatgatccatctctgaaaaatattgcctaacctaaatcaccagctttcctgtggcatggatatcctgtcttctgaagaactgtttttgcttttctggagcatgaatgtttgttgttttagagctctttgtattcgagttcatattttatccttatttttagtgcttttcctctaagtagcttgtggaaattatttctgagtaattgaagcttctgtgtcaacataatagtttgttgacataagaaacagtgttaagaaattatgcatttggaaaaaactgaaggtgattttttccagggaatttgtttcctggacttgggatttcttgtgcaaattttccccactagtttgtttttctctactattattttcataaaggagcagcaccagaatagtccttaatatcggagatagctgttgtgtcacttcctcatgctgacatatttactagagggtaaaattaataaccttctagtaagagtggcagtcgaagggaagggctcatctgacttctggaaacctatgttaaaatacagtgctttgaaccttgatcatgggctaattaggttggaaatgaaactagagaccagatacacccaaggaaacttttggtagttattttactttataagtcacaggaaatgcttgtgtactttcctgtgttccttaaatagttgcacttaaattacctctttgaaactgaatgaagagacagagagtattttttttctttccagaaataatacttgtttatacctgtaatagcaatgaaattttgtttcttttgagacagagttttgctctgtctcccagtctggagtatagtggcaggatctcagctcactgcaacctccacctcccaggttcaagtgattctcctgcctcagcctcccaagtagctgggactataggtgcactaccatgcccggctaatttttgtatttttagtagagaaggggtttcgccatgttggccaggctgatctcgaactcctgacctcaggtgatctgcccgcctcggcctcccaaagtgctgggattacaggtgtgagccactgctcctggccagcaatgaaacttgaggctatgcgaatgaaggattgttctaagagtgtaaactataaggcgaggaggattaaataaaaaaacagagataggctctcaaagcaccaaatagtatgattaacagattcttgtgtatcacatttgaaatgctttatgttttggctttatgttgatgagttgaaaacccgtgtttttgtttatgttgtttggtttttgtttttggctatcttatgtagaaaggcatgtaagggatggcattgattattctaaataatttttactgggaaagcttttccaagattttgaatagctaaatgtcaacttttttcttaatagtattttcatctcagcctagtgacgatgaatcaagtagtgatgaaaccagtaatcagcccagtcctgcctttagacgacgccgtgctaggaagaagaccgtttctgcttcagaatctgaagaccggctagttgctgaacaagaaactgaaccttctaaggagttgagtaaacgtcagttcagtagtggtctcaataagtgtgttatacttgctttggtgattgcaatcagcatgggatttggccatttctatggtaagtactcaaaaatgtgttatacataacctgatgggatagtgtagaggaacttaagagtgaggactcttgggatctgttttcagtgccattattaaatgtgtggccttgaaaacttgaagcctgtttgagcctcagttttctcattcgggaagtgggtgctgcctatctcacaaagctttttatttaaataaggtagtagttattaagaaatgtaaggataataaccatatatgtttgtgactcttttaaagttaaaagattggaagattaaaaatctgaaatgctcaccatgaggcaaatcatttacatttagaatcacacttaaactaagttgaagtgtgactttattaggaagattatttaaatcccagttgaaatagttacatttgaaatgtgtaaataataagtgttcattatttctactttgaggtttcatgtaagtatcatactttgtaactttcattgttgcttctgtgattcacatgaattgagagtttgtcatctttgttcttatactttgctctttattatgattgatactgtttatatatgttgattatcttatcaattcaaagcttttaaatgccttttgggtgagaagaggaaggtattctattggtgttactttactttttcatttaggagcctcacccattgactctctcctttgttttttggagacggagtcccactctgtcgcccaggctggagtgcagtggcacaatctcagctcaatgcaaactccacccccagggttctagcaattctcctacctcagcctccggagtaactgggataacaggcgcccgccaccatgcctggctaatttttttatatttgtagtagagacggggttttgccatgttggccaggctggtctcgaagtctttttttttttttttctttttttttgagacggagtctcactctgtcgcccaggctggagtccagtggtgcgacctcggctcaatgcagcctctgcctctgggttcaagtgattctcctgcctcagcctcctgagtagctgggactacaggcgcctgccaccacgcccagctaatttttttgtatttttagtagagacggggtttcaccatcttggccaggctggtctcaatctcctgaccttgtgatccacccgcctcggcctcccaaagtgttgggattacaggcgtgagccaccgcacccagccagtcttgaactcttgacctaaggtgatccaccccactcagcctcccagagtgctgggattataggcatgagccaccgtgcctggcctgagtctcttatttaaggtatgtgtttgtacaatgactggagtatttgaaacctgaactttgactctctttgctatattggaatttggggtagagacactttaaaaggaaatgtcttagatcttgtcaacttcccaaattttagcttgctttacattaactacttttagatttttttgcctgcataatccacagtcttaacttttcgttttgctagttcattcgaaaaatgtatttcttatgcaaagattgaagaattacatgtaccctatgtggatttgtggaaaaggtatattattcaacatagcgtaatgttagaataaacttaatgaagacaatacctgtttaaagtcagagcttgcaagatgtttcttgtatttatattaataaaaatatgtatttagatttctgatggatacctgcttgaatttaatactgagatactattatttgtatcctttcttgaaatttgttgtaataagatttgatttgaaatatatatttacctttatagactatctagctgaaacaatcaataagtatttgagtatgattttccctccacaagtaaacatgagggtaacaatggagaagatcttatcttaatctttttttcttacttcaaaaaaaattggatagtgatttaaagtgttaatccaaattgcattaaaacttcaccatttacatgacctttgaatattttgtagaaaaataattttacagaagtgctttcaaaatgagaagttacttctctgttatgtaggattttggttattagtaaaatttaccttaatagttaattaataaaacattgtcctttacttgatattagccttttcaggctatgaaaagcctggataacatattatgaagtaggcctacatagtaatgagattagttgaattaacatacaagacctgacctaaatgacttttccaagtcactacttaaaagacatcactttttttttttttgagacagagttttgctcttgttgcccaggctggagtgcagtgcgtgatctcggctcaccgcaaccttcgcctcccgggttcaagcaattctcctgcctcagcctcccgagtatctaggattacaggcatgcaccaccatgtccagctaattttgtatttttagtagagatggggtttctccatgttggtcaggctggtctcgaactcctgacctcaggtgatctgtccacctcggcctcccaaagtgctgggattacaggcgtgagccaccgcacctggcaagacatcacattttttaatttcagcactgatctcaacattctttggagttgatttcagagccgttttgaattttctattcaccgtaaagtcggtcattgcatcccatagtcattcattcactttataattttatttttttttgagacagagtcttgctctgttccccaggctggagtgcagtggtatgatcttggctcactgccacctccgtctcccaggttcaagcgatccttctgtctcaccctcctgagtagctaggattacaggcatccgccagcacgcctggctactttttgtatttttagaagagatgaggtttcgccctgttggccaggttggtctcgaactcctgaccttaagtgatccgcctgcctcggcctcccaaagtgctgggattataggcgtgagccaccgtgcctggcccgctttataataatttaacaataccatattactaattgaatcattctgcgtttgccagccctggtgccatacagatttgaattggcagatctatactgcagggaagagaaagctagcatgactaaggatagtgaggcagtgtactccaggtatttggttctgaaaatgcttccagaagttttattaaaaagtttcatgagaaagcagtctttcttattcttacatttgcttgtgactatcaacattccaaatcatcaatggagttcaattgatttggttttctcctcgagttgctttaagtattgatagactacccggctcttcggcagactattatataacaagatatctagatgctaagttatagagcacagaatgaattacagctcaaagggtacacttactgaattatcctggaggtggtaagatttaagttgtgtctaatcatagtaagtttatgatagactcaagggaaggtggaacattccagcgacagagaatctgataacctgaggaagacgtaatcttgggtttgggagcagtgaaaagactgcttggactacagcagagtgtaggtatttagaaataatggaataggtggggtgctattatagtcatcccttgatatacatgagggatgggttccaggatgcacccccataccaaaatccatacgtactaaagtcccttgactgcggacctcacatatatgaaaagtcacctctccatatacctggattttgcatcctgccatgtagttcaaactcatgttgctcaagggtcaactgtgtattgtaaaacgtcttggaatctagataggaaagtttgatttattgagtaacctgctatcaggttagagaaaaaagtgccggaactttttgctggtgtggaaaaggtggtatggaaaaatgtagaatagtcagtcagttaagagttctgtaatctggatatttggtgaggaggaccttgacaattggaataacagtgggaatggaataagggacattgaaggaataatggaaaataatggcaatttattaaattgtagaaaatgtgctagctgcttcacttttggagttggggttactggggaagttttgccagatgataaatagatggtaagcacagtttggtttggttttacccaaatttgttctagacaaatttggtttggggcaggtgttgaggtttgattttttttttttttccaccctacaaacaggccccctacacagctggatgtgttggctagaagctctgatgagaagttaggacaagatctgtagcttggaattcttaataggagtgatagcaaaatctgacagcgctgcaacatggtaaaataggcagcatttgtagacatgactaggccaaagattgtatcttgaggataagactttaggtagctgaaggtgcaggaaaaacccccaagtgattagtcagtaatattgtaaaaggccacattaagacagtaatcaagcatatgctatattatactatcttgaattcagacctcaacacagttttagattattttccagtgggcaaaggaattaatctcagcagtttgaaatagaaattgcagaagttttctgttactctgggaactatagataaatttcaacatttgaaaaaaatctgggattctgctgagtaattttgaaacaagcttgccgtgcttctataaattgagaaatctaaagagaggaaatgaattctaacacagaatatcatagctagactatgtaacagttacctgtaaattggcaaaagttgtttttggtgtttttcaagatgtattgaagacatttaaaataatttttatagcagtaagggaagaaatagtgatcactgcatcctttctttgtaaaattctatagtatctatactcagtagatgtaatgttaaggttgctaactgcattttgccttatttttgatttcgtggttttttccccccatacatgacgcatgtgaatagcatgtctgagtaaagtaacattttgatttttaggatgaaaatatgaagaatcctagttggtttaagattctttttttgagacggagtttcgctcttgttgcccaggctggagtgcagtggcgcgatcttggctcaccacaacctccacctcccgggtttaagcgattctcctgccgcagcctcccgagtagctgggattacaggcacatgccaccactccctggctaatattgtatttttagtagagacagggtttctccatgttggtcaggctggtctcgaactcctgacctcaggtgatccatctgctttggcctcccaaagtgctgggattacaggcgtgagctactgtgcctgtccaattcttttatcactgttgggataaggacagtgctactccagtacagatttaaccattgcagaattcttaacaaatttggccttgagaatggtgcacaatcaccattagccaattattttctgtgctgtactttttgggctttgttgtatccttaaggcaaaatagtaaaaaaaaaaaaaattttcatttttccttattgacttacgacctgtgaaaaaataccagggtttgttcatttctctgtataagggaggctgaaacactttttgtacttaaaagataaccccaaaacaacaaagctgagtgccttcaatatagtaagataagaacattacataaattgttctcttggttagagccatatgggctggcctgtgtgttttaatacaaaagatagtgaactgcttaaatgttcctgggtgtatgtaaataaatgtttttaagaactagcctacttggccttacttatattacgttaacgttcaccaccatttgatgaatgattgatcatgggctttcatttacttttctttttttattttattttatttttatttttagtagagacagggtttcaccatgttggccaggctggtcttgaactcctgacctcaaatgatctacctgccttggcctcccaatgtgctgggattacaggcgtgagccactgtgcccagcctcagttacttattctctatttatgtaatcagaatttttaaagaacggtgaaatgctgatactctgaatgaaatgattgtcaagattacataggttatggccaggcacagtggctcacacatgtaatcccagcactctgagaagccaaggtgggtggatcacttgaggccgggagtttgagaccagcctggccaacatggcaaaaccctgtctctataaaaaattagttgggcctggtggcgcttccctgtagtcccagacactcgggaagctgagtcacgagaattgcttgaaccagggaggcagaggttgcagtgagctgagatcataccagcctgggcaatggagcaagactcttctcaaaaaacaaaaaaaaaacaaaaaaaagactacatagcttatatgcagattaccaatattaaaaaatacataaaacatttaatgataatatttttgttacatacctttaataaaaatatttattttaggcacaattcagattcagaagcgtcaacagttagtcagaaagatacatgaagatgaattgaatgatatgaaggattatctttcccagtgtcaacaggaacaagaatcttttatagattataaggtatgtaccactaacagtatttaaaataattgttagtaattattttgcttattagttagagcttattgtgcttattagtgcagattagtaaatcagtaataatgctgatcattgttgaattgagtacgggtatttagtgtgtcctagtcattgtcatgagtgtaccatatagagtatttctcttattctcacaatatgtgaattaagcaattactaacttttataggtggaaaagaggtttagaaaagttaagtgactttctcaaggtagcaactggcagagctagtttgtttttgtttgtttgtttatttatttgagatggagtctcgctctgttgcccaggctggagtgcagtggtgcaacctcaggttgctgcaacttccacctcctgggttcaagcgattcttgtgcctcagcctcccaagtagctgggattacaggtgtgcacctctatgcccggcaaatttttttgtatttttagtagagacggggtttcactatgttggccacgctggtcttgaactcctgacctccgtctacccacctcagtctcctaaagtgctgggattacaggtgtcagccaccatgctcggccatggcctttttttgttttgttttgttttgtttgggagagctaattaattgattctaaggatttgacttaagaactcttatccctagctgctattttatagttaacatcttttataagtgacattttatatgtgtatatagacatttttattaacattattttcacagagcgaagcaaacattttcagttggcaggagggaaagaggagagttttggttttatagcctctctaagatcttctcttgatcaggggacatagctattcctttccattgtttttttttattctgatggaagggagagcatgttatctttttctgcctataactcacctagatctcgtctcacaggactgtactgtccattctgtcattacctaacacctctgtgggtttatgaggatcagcaaaatagctggtctcttatctagttttttcctaagaaggttgtagagagaaatgcagggtagaaaaataagctatgtctgggtgtggtggcaacctctcaaattcccaaagttggtaaccttgggaagccaaggtaggtggattgcttgagcccaggagttcaagaccagcctggccaatatggtgaaaccttgtctctactaaaaatacaaaaatcagccgggtgtggtggcacatacctgtaatcccagctttttgggaggcttaggcacaagaattgcttcaatctaggaggcagaggttatagtaagctgagatcacactattgcactccggcctgggccacagaggaagactgtctcaaaaaaaaaaaaaaaaaaaaaaaaaaaaggaaaaggaaagaaaagtaagctattgttttttccttcatttttccacccccaattctcacacttaatttatgtatgcttcccttacaggttatatttcttgaaaagacaaaaatactactttgtaactaaattgtatctttcagggaggtacttctcccatccaagtactaaccaggccagaccctgcctagcttctgccatcagaggagactggcggcatttagggtgttatggctgtagacacagggaggtacttctgaagttaattataaatagttcagtttttcctacctgtattagaagatgccattttcttcttcaccacagtgttttctggttccataggataacattggcttttggaaaactcaccacgcattaacctctagagagagtttgtagtttggtttgggtttgggttttttttttgttttgtttgttttttttctgagatggagcctcgctctgttgcccaggctggagtgcagtggcatgatctcagctcactggaacctctgcctcctgggttcaagtgattctcctgcctcagtctcccaagtagctgggattacaggctccctccaccatgcccggttcattttttgcattttcagtagagatggggtttcactatgttggccagggtggtcttgaactcctgacctcaggtaatctgcctgcctcggcctcccaaagtgctgggattacaggcgtgagtcactgcgcctggcctgttttttttgtttttgttttttaagagacagggtttcattcttattgcccaggctggagtgcagtggtgtgatcatggctcactgctgccttaacttctgggctcaagcaatcctcctggcttagcctctcaagtagctgcgaccacaagcacacgccaccatgtccaacttggtttttaaatttttcatagagatggggtctcactatgttgcccaggctattctggaacttctgggttcaagtgatcctcccaccttggcctcaaaatactgggattaaaggattgagccactgtgcccagctagagagtcagtttttacagatctcttatttctggctgttaagtgtggatcagatagattttgaagctctttggaagacctggtcaggatctgcttatcaatcacttatgtcaacatcatatttactgcacgaggaatgctgtactcttctttcatcctgtggcttcctgggcttgtgcatttcaagattatgccacaccagtgcatcagtctcttctactccacagtccactgctgttgggtagaagaaatttcttttctctcatacaggtagagcagatgaaatgactgtgattaaattttaaatctttatttgttctctgaagattaatgagccagaacgtgaagcagtcatttcagatagttatttttcaagtgtatattagtgtcattctcttaaaggacttgtatttttttttccttttttttttttcttatttctgaatccaggtaattgactgctctgactcaaatggggtaaccataacgtttgtgataaattaagccaaatttaagtatcattttgcagatttatacataggatatttttaactcgatgtataatgctttaagaacctgttgaaccatattgtttaagttttgactgtttttggttagattttgttactaatgcatatgtgtaaaagaaaattcaaatcagtcaaggatacccttattgaagcagtaaaattctataagccgaattgaccatgatttctaccacgggtgctgactttcagttatagtaatctatagattaatcttcagagttatattaatctgtacagattacagaacttcaaaaatttaccataaaattggataagttagttataaatgttcaagattcagtctgtcttctaacatggttatagaatttatggcttatctgtacttacttagaaatgttaattgcataggactaaaatgtttcaaataggactaagattgttagaatttcatcctttgaagggtacactgagtacaaacaatttttgaagccgtaagtcagctctcgatgtaattttacttttattttttatttgttttttttttgagacagagtctcgctctgtcgccaggctggagtgcagtggtgcgatctcggctcactgcaacattcgcctcccgggttcaagcgattcccctgcctcatcctcccaagtagctgggactacaggtgcatgccaccatgcccagctaatttttgtatttttagtagagatggggtttcaccacgtaggccaggatggtcttgatctcttgatctcgtgatccacccacctcagcctcccaaagtgctgggattacaggcatgagccaccgcgcccagctaataattttaccttttataaaaggtaatctcttttcctgattcagaaaagcctttagtcctgcaagctctagctaactttctgtcttttaaattcttccagccaggcgcggtggttcatgcctataatgccagcactttgggaggctggagagtcgcttgaactcaagaggcagaggttatagtgagccgagattgcgccactgcactctagtctggacgacagagtgagactccatctcaaaaaaaaaaaaattcttacaccatgctgtagttctcaatttgatgtagctctctgctctgcgttttcttgcatatgaaccttgtatttcccaatgtttatactattgaaaatactacccggggggttgtatctgtagaagtaaagacacaaaagcttatggggtattatatcttgaagatttaaattcagcttttaaagcatttgacattttatttcttctctttcaaaaaaaatgtagtcattgaaagaaaatcttgcaaggtgttggacacttactgaagcagagaagatgtcctttgaaactcagaaaacgaaccttgctacagaaaatcagtatttaagagtatccctggagaaggaagaaaaagccttatcctcattacaggaagagttaaacaaactaagagaacagattagaatattggaagataaagggacaagtactgaattagttaaagaaaatcagaaacttaagcagcatttggaagaggaaaagcagaaaaaacacagctttcttagtcaaagggagactctgttgacagaagcaaagatgctaaagagagaactggagagagaacgactagtaactacggctttaaggggggaactccagcagttaagtggtagtcagttacatggcaagtcagattctcccaatgtatatactgaaaaaaaggaaatagcaatcttacgggaaagactcactgagctggaacggaagctaaccttcgaacagcagcgttctgatttgtgggaaagattgtatgttgaggcaaaagatcaaaatggaaaacaaggaacagatggaaaaaagaaagggggcagaggaagccacagggctaaaaataagtcaaaggaaacatttttgggttcagttaaggaaacatttgatgccatgaagaattctaccaaggagtttgtaaggcatcataaagagaaaattaagcaggctaaagaagctgtgaaggaaaatctgaaaaaattctcagattcagttaaatccactttcagacactttaaagataccaccaagaatatctttgatgaaaagggtaataaaagatttggtgctacaaaagaagcagctgaaaaaccaagaacagtttttagtgactatttacatccacagtataaggcacctacagaaaaccatcataatagaggccctactatgcaaaatgatggaaggaaagaaaagccagttcactttaaagaattcagaaaaaatacaaattcaaagaaatgcagtcctgggcatgattgtagagaaaattctcattctttcagaaaggcttgttctggtgtatttgattgtgctcaacaagagtccatgagcctttttaacacagtggtgaatcctataaggatggatgaatttagacagataattcaaaggtacatgttaaaagaactggatactttttgtcactggaacgaacttgatcagttcatcaataagtttttcctaaacggtgtctttatacatgatcagaagctcttcactgactttgttaatgatgttaaagattatcttagaaacatgaaggaatatgaagtagataatgatggagtatttgagaagttggatgaatatatatatagacacttctttggtcacactttttcccctccatatggacccaggtcggtttacataaaaccgtgtcattacagtagtttgtaacatttgtagattggatagcatttttatgatttgatgagtttcttgtaaggttaccgtttctaagagttgtgctttatggccactgagagaattcagaataaattgaaagatggagtctaaaaattattagctgttacaaatggaacatttcattataacgtgatcactttgacttgagcaaatggtttaatttttatcttaaaaatcagttaagaatatataaaatcctactttggccaagtttgtttcttttcattatagtttatatgaaaagatcaccttaagtgaaattattttcctttaatcttttatgtatttattcacttttggaagctaggaatgagcaacacaaattttactctgaagtcagaagagctcatatataataattctaatgtcccacctattttcacttgtccattccatgtaccagcttagttatgatacttagtcacataattatctttgataaaggtagaggcacaaagaggcaaactaagcaagtcaaattctaatgtgtgtacttcataataattttttatccattttcatctttatattctgtaacatgaaacttacctaatcttcaaatgttagcttcattttttacctttgaaatacttaatctttctgaataaatataatgtgtctataaaataatgagactgattctggtgtctttagtcattaagctggtatctagtcctataatgaacaaaggtgaagctgccttgaggagacaagtgaaaaatttttgcttcaaaggagctcacaagctaagtaaataaatgaaattaaggtatggggcatggtggcctcaggctgtctggaggtgtttggaaaggcttcttgagtgaggtggcctttgaactgaacttagtttttaaagtagcttttggaagagaaatgaggatttgctatgcagacagggaagggaatttcacttaaaaggaaggtcatttggagatgtgaagatacactgctttaaggaagcagggtagagctggaggataagagatgcagaccatgaagggccccattttatgctaaaggttttgtcctgtaggacatggagaacttctgaagaattttcaaggcgggtgggataagattatattgtattttagattacagtagtccccccttatcttcaggatatatgttccaagacccccagtggatgctggaaaccagggatagaacataattctatatatactatgcatgaatttctttttccttctttacaatctcacacataggtttgttcttactatagatcttaccaatctcagcatacttttatttctcttgagaaccttcaccctttcacttaaaggaggcgctttatagcttctctttggcatatccaaatgccagcatcactgttgtattttggggtcattattaagttacttaatcatccttaatccttatcttagggatacttgaacacaaacactgtggtaggataacagtatatctgattaacagactgctactaggtgattaatgggtgggtagtgtaaatacacaagaaaaggatgattcacatcccatgtgggatggagcagaactgcattatttcattacattactcagaacaggcatataattgaaaacttatgaatttttttttttaattatttgagatggaatcttgctttgtcagccaggctggagtgcagtgacacgatctcagctcactgcaacctctgtctcctgggttcaggtgattctcctccctagtctcccaagtagctgggactataggcacgtgccaccacacccggctaattttcatatttttagtagagatggggtttcaccatgttggccaggctgttcttgaaatcctgacctcaagtgatccacacatgtctacctcccaaagtgctgggattacagtcgtgagccactgtaccccgcctaaaactgatgaattatttctgaaattttctatttaacattttcagaccacagttgaccacaggtaacggaaacctcaatcacagaaagtaaagccgtggatacggtgggactaatgtattggtagcagcctagaggattgatgggaaaggtatgaagctagaaggtggtcaatataatacagacatgagctgatgaacatctaaactgggactatactagtaggagaggaaaggaaaaaacatttggaaaatagtaacattgatatttcttgtgaaggagaagtagaaagtaacagtgacttctagatttctgggttgggtcatctgttgttggatagtagtaccactgagatagggaattcaaggtttggggcaagggtaattggagatgagaattgtgtttggaggtaactactgacattcaagtggagagggttagttggcagttagttctatggtcatctcttttgccgagactgtatatttatcagactcctgggagaacaccaacatccatggggttgtagggaaggctaaggacaagagtggggagtggtaccttgaaaatccaaaagccatctcaagtaaaaggaataaatgtgtcatgctttttaaaaagttgatgtgcggaaaatgttttcttggcttggaaactgggcggccaggggatgacagtatggacttccagtgaagtagtgacggaagcctgatcatagacattaaggaaagcggtgtaggtgttgtgagcttttgctgtaagaaaaagttgagacttttgttttgctttgtttgtgagagatgtgtatgtatttctgctgagtgataaagccagcggggagggactgatttttataggaaaggaggaaaaataatggaaacacatctcattattttattgtcacatttcttttctttgttatcttttgagtgtttcccttttttgccagtagagttattgtctattttttctttctataggacaaaaaaactaatacagactcctttatttttatatggatatactaggattgtaattcagatatttaatatcttttatcagtgttcagatcatagattaatggagaaaacatttaaaattgttttaaatttaaatacattgaactctaacatagatgaaaaatgtgtttactgctttcagtcgacctgataaaaagcaacgtatggtaaatattgaaaactccaggcatcgaaaacaagagcagaagcaccttcagccacagccttataaaagggaaggtaaatggcataaatatggtcgcactaatggaagacaaatggcaaatcttgaaatagaattggggcaattaccttttgatcctcaatactgattcacaattgagttaaattagacaactgtaagagaaaaatttatgctttgtataatgtttggtattgaaactaatgaaattaccaagatgacaatgtcttttcttttgtttctaagtatcagtttgataactttatattattcctcagaagcattagttaaaagtctactaacctgcattttcctgtagtttagcttcgttgaattttttttgacactggaaatgttcaactgtagttttattaaggaagccaggcatgcaacagattttgtgcatgaaatgagacttcctttcagtgtaagagcttaaagcaagctcagtcatacatgacaaagtgtaattaacactgatgtttgtgttaaatttgcagcagagcttgagaaaagtacattgttctggaatttcatcattaacattttataatcttacactcacttcttgtctttttgtgggttcaagagccctctgacttgtgaagaatttgctgccctcttaagagcttgctgacttgttttcttgtgaaattttttgcacatctgaatatcgtggaagaaacaataaaactacaccatgaggaaaactaaaggtctttatttaaaatctggcattgtattaacatgtaattttatactatgtggtattttatacatttcctcagtagtgatatttggtaaagcagttcatacagcttttttctaagttccatgaatcttacccagtgtttaccgaagtatttaagcagcatctgaatatttccacccagcaatgttaatttatctaggaaagttcagaatttcatcgtcatgttgaatttcccttttaacttccgttcatagacatatatgtgacttccaattcgaccctctggcaagtgagtgtggaagaaaacagcagttcttttataattgcttgaaattaggaaagcgcttatttcctagaagcaaataaatgtttgagtaaataaaggctacattttgctgagtactgtttcagtcaattttagaattgcctattctaattgctgtctacaactttttgcttaataaaagaattaacttataggaagtaaaatactttgatagttacagaactggggctaattttgactggagaaaagactaattatgaaatgtgggttgaaacagaccataaaaaggttatattatctatttacctgagggtttcctaaacctggggtaaaattaaaatactgagttttcctctttttcatgtttacttacctcttccaaaatgccgaaggtgatcaagtgagtaggcaatgatgcatcatcatgaaactctctatgtaaccagtttaagggatttaggtaaaatacatctgcttcatcaagataatgactttttccagtcaggtctggcgggcactggagaaatctcatgggaagtgggcagtgaacatggctatgaaaagaaaagttaccataataaatacaagtaaaacatgaaagttttcattagtcttaagatatgtcatttatacttagcaaaatgtgaaatctgaatctgtaaactgtgttgcagttaagtgaagaagcaagcctttaactcattttctcaaagataaaaaaaacatgattcttataccatgtacctatgcagcagcagcagtataacctataagcccaaactagtgcatagaagtccatttcaaggagggaagtaggtcagtagggggactcaacattctgtagcattggtattctgttccggaaacataaatggtcctcaggaccaggtaagttaacttgttaataggagagtaattctccataggtgtatttgataaagtaggaaactgtgatcataatcagaaaggattctttctctcttccctttctgctctgataggggatggatcacatacttatatggcaagttttttttttttgaagacaagagtttcgctctcgtcacccaggctggagtgcaatggcgcaatctcagctcactgcaacctccacctcccagcaattctcctgtctcagcctcccaagtagctgggattacaggcgcccgccaaccacgcctggctgatttttgtatttttttagtagagatggggtttcaccatgttggccaggctggtcctgaactcctaacctcaggtgatccacccgcctcagccttccaaagtgctgggattacaggtgtgagccaccacgcccagccacatggcaagtttgaagtgttttttttttttatttaagatgattgagaagtagagagcaacatatgtccactgattaacatccaaataagtaatggttttattgtggcatagaaagactagagtaggccgggtgcagtggcttatgcctgtaatcccagaactttgggaggccaaggcatgtggatcactggaggtcaggagttcaagaccagcctggccaacatggtgaaaccctgtatctactaaaagtacaaaaattagccgggcatggtggtatgtgcctgtagttccagctactcaagaggctgaggcaggacaatttcttgaacccagaagacggaggttgcagtgagctgagatcatatgccactgcactccagcctgggcgacagagcaagagtctgtctcaaaacaaaaaaaagattagagtataaaaaagatggattcatttctaataccagttatgacaatttaagccagttacttaggtcatgcaaaaagccatgtttataattgccttaggaaaaattaaacctcactgatacaaacatttaaaatttccagttagaaatggagttagtaattatgcaggaggtttatttatgaaatgcataaaaattaaatgtacacaagacctaatagtgatttatttattggaaatgaaggtcttctttcaaagggaagcagaaattcctcagaaaaagctgccggcctactctagtctttctatgccacagtaaagccattacttatttggatgttaatcaagggacagatgttgctctctacttccaagtctgatgcaaaataggcatcagactttcctctcttgtgtgctttgaaaaggtgtggagaacttggtgaactattcaaagcctaagacatcaactgactacagttaagtataaagaacatcttggtacatctaggattcctgttctcctcagtattatccctaatgtgagcatggagagtgaatgtccctttttcagccacatatacatatagtagggaatcctcaagttgaaaggatacttgatgtcttccttttagcctgtggtttttgctaggatctccatttttctaaactgtatattaattactggcattaatgattagttaaataggagtattccaagtgttaaattcccctaaaatgataaccactgtaattaaattaattgaacgtgcataggagacattaactccagattgtttaaagccagactgagttaaatagttatagattttatgtatttatttatttatttttaggttgagtctcgctctgtcgcccaggttggagtgcaatggtatgacctcagctcactgcaacctccgcctcccgagtagctaggactacaggcatgcgccacaacacctggccaattttttttgtattttagtagagtcaggggttcaccatgtggcccaggctagtctcaaactcctgagctcaggcagtctgcccgcctcggcctcacaaagtgctaggattacaggcgtgagccacaacgcccagcctggtatataattttctgtaggaaatctggctcctttgcccctataccattactgggtaattagtaactaatacagcttcctgaaataaagcacaggaacaaatggatgtctttttaaaggccatacatagttgaggcaattttagtatatttcccagaaacacttaattcaagcattagaatatcatctagtcgcattccctaattttttacaggtgagacaactgatgtccaaagagattaaaaatctagtttaaggccgggtgcggtggctcacgcctgtaatcccagcgctttggggaggctgaggtgggcggatcacctgaggtcgggagttcgagatcagcctgaccaacatggagaaaccctgtctccaataaaaatacaaaattagccgggcatggtggcacattcctgtaatcttagctactcaggaggctgaggcaggagaatcgcttgaacccgggaggcagaggttgtagtgagccaagatcacgccattgcactccagcctgggcaacaagagcaaaactccatctaaaaaaaaaaaaaaagaagtctagtttaagatgagggcaataatccagtagtaaatctggactaaaatccatgtttttttttttttttttttttggagacagagtcttgctctgttgcccaggctggagtgcagtggtgcaatctcggctcactgcaaactccgcctcctgggttcatgccattctcctgcctcagcctcccgagtagctggaactacaggcacctgccaccacgcccggctaattctttgtatttttagtagagacggggtttcacccagcttagccaggatggtctcgatcttctgaccttgtgatccacccgcctcggcctcccaaagtactgggattacaggcgtgagccacggcgcccggcctaaaatccatgttttaagattaattcaaattgtgtaaagatgtatcacagagatttgtagtaaggtaagtaatacatgagtttctttactaagaagactatcagatacctacaaattgttttatactgatttaaatcagaattaggtgtaagtaaattgtttttaaagaataataaagtttacggccgagcacggtggctcacacctgtaatccccgcactttgggaggccagggcaggcagatcacgaggtcaggagatcgagactaccctggctaacacggtgaaaccccatctctactaaaaatagaaaaaattagccgggcatggtggcaggcacctgtggtcccagctactcgggaggctgaggcaggagaatggcgtgaacctgggaggcggagcttgcagtgagccgagatcgcgccactgcactccagcctgggcaatagaacagactccgtctcaaaaaaaaaaaaaaaagaaaaaaagtttatttaaaacattttctttgacacagttcaagtggtttttaaaatccatcagtgaataaatatgtgggagtttaaaatttatcatttgatcagtgaccaattaattcaaatccaggaatctgaaagggaattttaaagtagtcagtttcagtttcttaacagcatatagtggaacatcttttattacctgtaataaggagtagagtggcaaggcatcattataaatattgaagctgaagatttattgggattgttgtaacaaactttttgaatatgactcatgacatcaagagtacctcgttgatgaactaaaccagtataaagggcgaggaacaaatttgataaaaacaggaaacttagagctggtttcttccatgttttcaggtgggttaatgagtatcctatatgaagatagacatattgaaccttaacactgacaatcttttgttatgttaactgcaaatacatctgtaatttttaatggaaatagcaaaaactgtaatactgttatagacaaataagaaataagcaataagtcatatctctgaccattcaccattcactactgtcatctaggtcttgactttttctagtaaaaggtattgaggtatagtggaaagatgatggttatggagtccaacaggtccagatttgaattccagttctagcacaagtcgttttactttgctggtcattgggataatagtaccttattcaagtgttagaaaaaatgtccagtgactgccctggtacatagtagttgctaaataaataggacttttaattattccatttacctacctctttaatctcccaagcactaataattggtattttagtgtgtaatacgagtttcaataatgtgtcctcttaggaaaataagccattgggcacctgggagattttccgtaagtatatgagtggaagaaaaggtgctatgtcttacattttttttcccacaacagtgggaaattttgttttcccactacagtcaggcaatttggagatgctgaacaatttttcttttttgatttatgaaagggattattctgtggggcaaaacaaaacaaaaaaccaaaggaaaaaaccaagtagaatgaatagacgggtataaaaggataagtaaaagtctctctcccctctgatagtccttttcttcagcaacagccactattaatggacatattaggatctactgagaatacaaagaacaggcctttccactggaaaagattaagcttattagtaagcatgacattttgttcataatttacatacactctttttatagaactagataatagttctttttgaacttgggtattcttcatccaaaccttggtttgccca

SEQ ID NO: 10 - DNA, 17386 bp, Homo sapiens
  human DYXC1 chromosomal gene region, nucleotides 200001-217386
aaatgttagtacaatactgaatgggaccaaggcagtgttttttattttgttcctcctatgaatatgttaatatcatccacagttcatacctcatgagttggaatatcacatgtccatgtcacctagtattttttaatctacttgaaatagttctgattaaagaataatctgaaaagtggtgtggtgaaaaatgtagcaagattaagaaaactaagcctatcacttaaatctacatttataagatgtttgaaatggtaaaatgaggaacctactaaaaggaatgataattagaactagctttatttgaaataaaatagctgagatgagaggattgcttgaggccaggagttcaagaccagcctgggcaacatagtgagactccatttaaaataaaaaaaaattagccaggcgtgatagtgcgcacctgtagatccagctactgggaaggctgaggcaggaggttcacctgtgctcatgagtttgaggggacagtgagctatgaccacgccactgcactccagcctgggtgacaccctgtctctacgaaaataaaaatgaataaaatagggccaggtgcggtagctcacacctgtaatcccagtactttgggaggacaaggcaggcggatcacttgaggtcaggagttcaagaccagcctggccaacagaggtgaaaccccatctctactaaaatataaaaattagccgggcgtggtgtcagaggttgcagtgagccgagatcacgccactgcactccagcctgggcaacagagtgaaactgtctcaaaataaattaaaaaataggattctaaaagttcaaccatctgtaataaccccaatcacagacacagcagatccccaaatataagttgaagcacttaacttaggttggtaggctaaaacaacatctactttagtaggctcaaatacaaaaagctacattaaagccttgtctttgtttgtactgctatactggaatacctgagactggctaatttatatttacttggctcatggtcctgcaggttgtacaagaagcatccactggtgagggcttcaggctgcttccactcatggcagaaggtgaaggggagtcagtttgtgcagagatcacatggtgagagagcagaagcaagaaagtggaaggtgccaggctcttttgtacagccagcactcgggggaactctcatggaacctaatataaggagaactcacttactactccaggatggtgccaagccattcatgaaggctccacccccatgatcgaagcatctcccattaggccccacctccaacagtggggatcaaatttcaactaaggtttaggggacaaacgtccaaactacagcaagcccatcctacagcaaggcccgagaagatacagtttgttctcaattatttgaagatacttacaaggtgagcatgtgtaaagtgattcacttattaataatttgttcattcaaggccgggggctgtggctcacgcctgtaatcccaggactttgggaggccaaggcgggcagatcgcgaggtgaagagatcgagaccatcctggccaacatcgtgaaaacccatctctactaaaaatacaaaaattagctaggtgtggaggcgcatgcctgtagtcccagctgaggcaggctgaggcaggagaatcacttgaacccggcaggcataggttgcagtgagctgagatcgtgccactgcactccagcctggtgacagagcgagatgtcgtctcaaaaaaaaaaaaaagaaactatggcactaggaatacaatagtataggtgggtgacacaaaatgggaatacttggtgatcaagactgtgaaaatagcactctgggtagaatgctggaagataacatgcttgtgtttccagcatcttcagtactggtgagacagagcgtctcttaagttaggtaatgcttgccttacatgatggtgattttaactctctgagcttagctcattttaggatggatcattttagtgagaactgaatctccatattacttagagaagtaatataaagaaaatgctgggaagagaaggaacagctgaacaatgccagggagagtcaggtgatagcttagttttcttggggacagtggatactgaaacagttgccattcaaaattgtcttcaacagtattatggatccgatgagcttttgtgtaatattctgggagccttactacatgtcacagtattatttctaagaaaaaaatatttcaggttccaacttaaaaatgaagtttggggataaaaccttatttaaaatagaactgtctttatgattattatttttttgagatggactctcgctctttcgcccaggcgggagtgcaagtggcatgataccggctcactgcaacctccacctcctgggttacaagcgattctcctgccttagcctcctgggtagctgggattacaggtgcctgccgccatgcccggctaatttttgtattttaagtagagatggggtttcaccatgttggccaggctggtctcaaactcaggtgatctgcccgccacagcctcccaaagtgctgggattacaggcgtgagccacagcgcccagccagaactgcctttatttacatgtttagcatcctataagcattactatgcatttgacatataaagaaaatatctttataatttcagttatcatcaaatgacttgtgcacatgttagaaaagaacccaactttagagtataagtcaagagtcctgaatttaaggcctctgaggggtttttaaaccttctggcactgaaagcttccaaaggggatttcagaaatgagttgctaaaggaagggttggaaagattttccagttccaatgctagactgagtgaaccacaagagaagcctaaggtccatggttttcttatagaccagtagtattgtttcatcaatatttaaaacaaatgaacaagattccagtaggtgacatccagtaagtctacttaatggcacgcacaaatttggagatggagttctcactgaaataatccaccctcaaatgacctagcttcagaaagtgatcatccccaccacgtaaatgatgtgtatcccaatcactgaagatttaagactgtgtgtgtaccccagtgactaaagtataggttccttcctttcacatagtaacaacccattgctaactggatgctaaacatcacccctaaatacttaatttgctcctgttatctgggcctacaaaaagatacttgaaagtcggtatacagaaaatttctggaagaactgcttaagaaagtgataacaatagttacctctggggaatgggactagatgacttgaggatgacacactatttttgtaccatcggcatttatttttataataaaaaaatacactgggctcctatcagatcatgttcatcagactactgttacagaaatgtttaaaatccattacatctcagttgactactcttcaggataaaatctaaaaacctgagcatataagaagatacttcatgaggtggttcctttataaagccatatctcttaatcatcatgtattatttagcttgatatacacactttaaaaattttggatatctagcgtggcattctgtattttccagcaatcactaatacgttccttcttaccaaggtaaggttcacggtaatagagaggatgggagtatctgagactgtacattgctgattccatcctggttttgacaagaattatcaggaaacctaatgttaagaaaaaaaatccctaccacattttaccaattagagagatatctgagttactgtttctatttttaaatactctttgagaaagcttagtttcagtgtttagataagaacaacaaaaggtggttcaattctggaatgggacatacagtagaatggggctgggaaagagagactttgaactactaaccataatgagggtactgggcacagatgaaggtatcaagaagaattttttttttttagacagaatctcgctctgtcacccaggctggagtgcagtggtgccgatctcggctcaatgcaacctccacctccagggttcaagtgattctcctgtctcagcctcccaagtagctggcattacaggtgcgtgccaccacacccagctaatttttgtatttttagtagagacagggtttcaccctgttggccaggctggtcttgaactcctgacctcaggtgatccacccacctcagcctcccaaagtgctaggattacaggcgtcagccactggcagctggccaggaagaattttatgttgctattttgttaaggaagtaaagccatctttccatcttcgatagaattcaacagttaatgcctaaaacaaagacctttaagtggcattataaatgttttagagatatcgagataaataccaaaataaccagctgaaagaatgcttaagtgattgtttttggggagaggaaatagggagagcaggggaacaatttttttttttaattccttgtggaatgtcttctgaaataatgtacatatgcaattttgataaaaagattagaatatggggccagctgtggtgcctcatgccgtaatcccagcactttgggaggccaaggcaggtggatcatctgaggtcaggagttcgacaccagcctggtcaacatggtcaagccccaactaaaaatacaaaaattagctggctgtggtggcatgggcctgtaattccagctactctggaggctgaggcaggagaatctcttgaacccaggaggcggaggttgtggtgagctgagattgcgccactgcactccagcatgggcgacagagagagactctgtctcaaaaaaaaaaaaaaaaaaaaaaagataagaatatggaaaaacaaagtggcccagtaagagagaagtctatattagccactgtggtaaaaacagataagcagacactcaactcagaatcagtagtgggtgctgaactctgtcagcacaaatgagagattcaaactaagaatttaattagtgttgctaaaatatcacctttcttttacagataaggaaaatgaggcacattgttgaacaattcactaaagatcagagccagtcacaaaatatattttctagagttgagtcctattttataggactaagggaaattcaaattaggcaagagaagttaccgtagactgaaaaaactgctgcaattatttttcttcctgtgtcccatcccactatgaggttggcatttatttccccacctcttgaatctggcctggcccagtgacttgctttcatgaaaagaatgtggtggaagtgacagtatgcaaattccaagcccttgtctcaaagggccttgcatatttgctccctctcttggaacccggcctatgccaagtgaacaagccctagctggcctgttggaggaagagaaaactcagaagagctgagaagacttggcctacagctagttgaaccccagacatgagagcccagcctaggtcaatagagctatccacccaatctgcagctgtgtaagtgagctcagtggagatgagaactgcccagttgaaatgtagacctgtaaattataataaatggagttttaagccactacagttttgaggtggccttttacacagcaatggctgacagagttgtcatgtttacataaaaataggttaaaacaagtcacacaaagcagaataaggctgaaatttgtggtataactcaagatttcaagtagttaggaaagattcaaactgggtcactaaaacatcaagagctccaaagagaactgttctaaatgtccagagatttctgtctaggtgacatcactagacagaactgatcctactgttggaacctcttggcattaaacagccctgactgttagtggaagtacaaatctcctaggaattagagttttttgacccactctagaaccttaggtagggtgtggaggaaaagaatgcatcctgtgaggagatcaaggggttttctaggttgctgtcttttagggatccttctaagtagacagaggatggaagaaggggtgattcttacctattagaaggggctctctttacccctcatcagcattttatctcaataaactgtgaataggccctggttacagggatcttgccacttaaagattcaatcttttagactggcaatgaggattcagacaactcaatctttgtgtaaatacttggtaaagcaacaggacacagaagaggaatgctggaaaaatctggtttatgaaaacagaaatcaaaccaagttactaaccaacctccccgtcccctccaggcacacaaaaacatttgcctttgtactctgccaatgcttgatttaattataatacacactcaagtggctgtaaaaaaacccaacagaacagaaaccatttaacatctgaatagtgatttatgaacagatttcctctgacaaatggggccagctattcttaaggcttgccaaatagctgttagatgtctaaatttttgtatgtgcctgcgtttgtatgtggttacactgtaaagtaatgacagactgaggctttgatttcagaacttcagatgtatcaaaaatacagatttttaatatttgattttatatttgtatgtttatcttcttaaattgaagagattatacatctcagatctgtaggagttgatgtatacttggtcaaacattgcctaactcaatgtttttctttttttttaagtaaaaaataagtttgaaaactgtaactccctaggttttttccagcctccagttccagttggctagtcatttcttctctctcaacgctgtgattttttttaaggtcttaatatttgaaggaagtcaacagtcatttattccgaattaaacttgaagttaataaagtttcaattcgtaatttttccaaaccaaccaatgtaaaaacccagattttcctgaattgagtcatgtaaggatttttgtaagtgacaaaaaaatacatgttaagactgtggaaaaatggagaaaaggcctgaataaaatcaggagttaattacttaaaaaatggcatatgccaggctgggcgcggtggctcacgcctgcaatcccagcacttttgggaggctgaggcgggtggatcatctgaggtcaggagttcgagaccagcctggccaacacggtgaaaccctgtctctacttaaaatacaaaaattagccgggtatggtggcggatgcctgtaatcccagctactcgggaggctgaggcaggagaattgcttgaacacagggaggtggaggttgcagtgagccaagatcgtgccactgcactccagcctgggcagcagagtgagactctgtctaaaaagataataataataattgcatatgccttagagtgagttttaaaagtgtaaatgttttgtgtgtaaaagcattaaaaactagtttcaacagcagtaaatacatttttcctttgtgaaaatgaagacccttacagtttaaccataaacttaaaatagctaatctaaaaacagacatgttactttactttttagtgagaaacaccaccaccaaatagaaacaccaccaccaaaaactgctttgggtattggatatttatgtgggttttttgtttgtctgttttttgagagaaggacttgccatatggtaggctggtctcaaactcctgggctcaggtgatcctcctgcctcaggcttctgagtagctgggattaaaagtgcacaccactgcactgggctaggatctttatgtttttatctcactttagaggatataggaaaaaagatatttggactacaaaaagggaatattgcagtaatataattaatctgtaattaagtaattccttattaacctagttgaaaatagacatagtttaatgaggatgtgttgtctttagactatttctatataatttattttctataacaaacaaaagcacttaccacagaacaccatacagaatggtaaaactggataaataaacctgaattctttgtggctcaacatgctgtaataaaaacaaatattattaaaaaatattaattatcatgttatatgatagattgagaaagtaatatatttaatataaaatatatttaacttgatctatttattttctaagtattcagactgaatttcttgcttcagaaattcatcttattttttagttataaggtgattacgatatggtaaaactaatcataaaatatgatgtcaatttgaaattacaaagtacagttacattctaaaaacgggatttctatttcaatccaagtaagtgaaattagtcaaatttggaataaggatacatacatttttattttagaatgtatttctgtggtccttcaatttaaaattttggtagtattgattgactgattgactgagacagggtcttgttttgtcacccagactggagtgcagtagcacaatcatagctcactgcaaccttaaattcctgggctcaagcagtccttctgcctcagcttccccaatagctaggactacaggcacatgccatcatgccctggtaattttaaattttttttcagagagagggtcttgctatgttgctcaggctggtcatgaactccttgcttcaagcaatcctcccaccttggccttcaaagtattgaaattacaggcatgagcaactgtgcctggtctgttttttttttttttaaagcaaataaaattagtccaagttcttaaaaattgagtgtaatccttattaagcaaactacatatctaaagtattcactctagtgcctccataattactactacttgtatcttctaagataattcccactaacaaaagttaatggcacctccaaaacataaagtttactttttgaagaatttaatttgatttcttggtaacataaaaaattttagccttttgaacaaataaaatcttacctataaacaagcagtgtccacagcacagtcaccaaaagtatccggtatctctttggtgctagatagcagccatgaataaagaagggtaagtgagtacccaagataactggaaatccttgactgaagtaccagtgccatggatgagaaccataaaatgttcccaagttctgcagcacgttaaatttcaaaaaattaaattgaaccagagtccactggcaccgttgaaggaaaaataaatagaaatgtgttagtgccaagtagtaatctttggcattaggggattgatacaattttgacattaaatcagaattgaagtctaaaaaccaggcaatagtttacattaataattttttttttttttttttttttttaagatggagtcttgctctgtcgcccaggctggagtgcagtggcgcgatctcggctcactgcaagctccgcctcgcgggttcacgccattctcctgcctcagcctcccgagtagctgggactacaggcgcccgccaccacgcccggctaattttttctatttttagtagagacggggtttcaccatgttagccaggatggtctcaatctcctgacctcgtgatctgcctgcctcggcctcccaaagtgctgcgattataggtgtgagccaccatgcctggcctaataattttaattaatgactgtatgaaatccttcttaactgttattttttcttctttaagtattaatgtgttccttgttaatttgtaaaactagatttttgagattcaaagtgagaatcaaacttatattttggcagtctaagtttgtgtttttatttcaagtttttggggtacagatggcttctggttacatgaataagttctttagaggtgatttctaggattttggtccacccatcacccaagcaatgtacactgtacccaatatgtactcgaaataagaatttctcacccctctctcacccttccccaccctgagtcaccagagtccattatatcattcttatgtctttgcatcccatagcttagctcccacttataagtaagaacatatgatatttggctttccattcctgagttatttcacttagaataatggcctccagctccatccaagttttatatccagttaagagataaagaggatagtagacatatttttcggactgtgtggtactttacaatgtgacatattacctgtgaagataaagctttgaaaacattacaatttgaattacagcgtaaagctgacaataatgacagaaacagcaacaggtaacattcagtgctcacttaataattccctgccttaaaatgtactctcctgaatattagctaggagtactaattcttttatgtagaaagcacaacaaattgaaccaaaggcttattaggatatgcctagaagtcttttgtaatgtatcccatatcatttaaaaaatcatttgcctctgttctaaagtattaatggattaaaaataaaaatagctgttagcttaatatacttttacttacttggccaaaaaaaatacgatcaatcatcagagacaaactcaaagtaacaaagctgaaaaacaaaaatagcatagtgattcacatttggagcttgctgaaaatccactattagcatatggctttgtacgtgccttgcatcaaaagccactataaaaatgctttacagtttcaccaactgaatatagatttacacattttgaaaggtatctactgaaaaagatcagtagataagattatataatcgattaatattgtaaaaaatatattaataaaaagattattgaaagactactgctgtataacaaattaccccaaaacttgttaacttaaactatgaatatttaccatttcatagttcctgtggatcaagaacctaggtgttgcttagctgggtgcctctggctcgaggtcgctcatgaattgtagtcaagctcttagcaggggctacagtttcatctgaaggttcaattacaggtggagagatcagcttccaaatttgctcacatggttgctggtggggctaagttcctcaacatatggccgtttccttaaagctgcctcatgacataattaactggcttaacccagggtaagtcagccaagagtgagtgagacaggcacccaagatggaagccataatctttttatttatgtattcttttcttttttttagagacagggtcttgctctgtcacccaggctggagtgcagtagtgtgtgatcatggctcactgcagccttgacctcctgggctcaagtgatcctcccacctcagccttccaagtaactaggactacaggtgtgtgccgccatgactggataatttttggattttctgtagagatggggtctcactatattacccaactggtctcaaactccgggcctcaagcagtacttccccattggcctcccaaaatgctgggattacagatataagccactgtgcctggccaccatagtcttttcataacccagtcttgtaagtgatatcccatcccttctgctgtattctactcattagaattgagtcgacaaatctagcccatgttcaagggggaggggattatacaaagacataaatagcaggaggatgggatcactggaggccatctcagagactgcctccaacaaacacatatacagaaaaatacgctatgaaggtaaagctctatgaatcatcactacatgaacacaactgtgtacgtgccatacagagcaggataaagagtattagcagcaccaccttgtccctccaatcagtcagtccctacctcctccccaaaagtaacccaaaggtaacccttcctcttcccaaaggtaaccactatcctaatttctgataacatagatgagttttatctttttttgaattttataagaataggatcataccatatgtgctcatgtctaaattcctttagtcattgtgcttgtgagattgaaccaagtttttttgcgtgctgtagttcattcctttacaatactgtgtattgtaactatgtataccacactgtatgtattccttctattgatggacactgtgcttccatttccagctattgtgaatagtgctgcaatgaacattcttttacacatcttttggtatccatatatgtgcatttctgttagacatatacctaggattggaattggtgggccctaaggtagcggtccctaacctttttggcaccagggtctggtttcttgaaagacagttttcccacaaacaggttgcgggggtgggagtggggcatggtttctggatgaaactgttccacctcagatcactggacattagatgctcatacggagcacaacctagatcccttgcatgtgcagttcacagtagggttcacacttctgtaagaatgtgacaggaggtggagctcaggcagtaatgcttgcttgcttgctcacctcctgctgtgcagcccagttcctaagaggccatggactggggccagtccacagccttgggactggtgacccctgccctaagttgtaagtgcaactatgtatttatttagcttttgtaagtacttaaaagttttccaaaatagtgtattagtttgcagtcccacaggaatatataagttccacatcattttagccatttgggtggatgtatagtatagtagtatttcatggtggctttactttgtttttccttgataacaaatgaaatgagtctctttatatttattggccattttaatattttttgaagtacctgatgaaaccttttcaataatttttctatgagttatctttttcttatagatttttaaaagttttatatataatctggatgtaaggttttatttttgagatggagcctcactctgttgcccaggctggagtgcagtggtgcaatttcagttcattgcaacgtctacctctggggttcaatcgtttctcctgcctcagcctcccaagtagctgggattataggcatgtgccacaatgcccagctaatttttttgtatttttagcagagacagggtttcatcatattggccaggctggtcttgaactcctgacctcaggtgatctgcctgcctcagcctcccaaagtgctgggattataggcgtgagccatcgtgcccaaccactagaacactttaaatcactgggtaagaattacaatccctaactgatgatgattcaacctacgatttttcaactttaaaatggtacccatacagccattctgtttttcactttcagtacagtattcaatgaattacatgagatattcaaatattttattatgaaataggctttgtgttagatgattttgctcaactgtaggctaatgtaagcgttctgagcacgtttatggtaggctaagctgtgatgttcagtacgttagatgtgttaagtgcatttttgacgtagaatattttctttgttatttattttgtttagagacaggttatcactctgtttcccaggctggagtgcagtggaatgatcatagctcactgcaaccttgaactcctgggttgaagggatcctcctgcctcatcctcccaagtagctaggaccacaggcgtgcctagtgccacctatacttggccaatttttaaatttttttgttgagatggggtctcattatgttgctcaggttgttcttgaactcctggtctcccagggcactgggattacaggtgtgagccactgtgccttgcctgacttatgatattttcaacctgcaatgagtttatcaagatgtaaccctgagtcaaggagcatctgtattgttatatctttttcccccacagactttgactttgagagcagacactgtatcttactcacttctgaattatcaaatgctggatacaaagtaggttaaatgcttattaaatgaataagttattatcatgctgctgatgagattaaaaaggctttctatctccctcagtggtaaagccgataggaactgagcccctgctatctctctgacctcattattacattctcccaaactcactctcttttagccacatcagtctccttgctgttccttgaacatgcccagcatacttagtacttggctggcccctttgcctggaatactctctcatcaagtaactacatggttcactctctcaactcctttaagttttggctccagtgtcaccttatcatggtgatctttcccaactaccctatttaaaaataacagctcccccattaccccaagcactggcattctatccttcttatcccacaattatttttccctgtattaccatcacttgacaaatatatatttactcttatctttttagctgtctgtctctccctactagaatgtaaacttcatgaaggcaggtactttttttggttaaactgatgcatctcctacatctggaacactgcctggcacatagtaggattaaataattcctgaatgaatgaatacatttaatgcaatgttattaatacatctgagaaacgacaacagtagcaactgagatgcaagataatttagtccttttcagattgctacccgaaaaatacttgtgactagtgaatactagtacagtcatgcattgcttaacaatggggatacattctgagaactgcatcattaggcagtttcatcattgtgcaaacatcatagagtgcacttacacaagcctagatggtacagcttacagacatctaggctatatggtatagcctactgctcctaggctacaaacctatacagcatgatagtatgctgaatattgtaagcaattgcaacacaatgctatgtatctgtgtatctaactatagctaaacaggctgggcatggtggcccatgcctgtatttccagcactttgggaggccaaggtgggtggatcacctgaggtcaggagtttgagactagctggccaacatggtgaaaccccaactcgactaaaaataaaaaaattagccaggcgtggtggcaggcgcctgtaatcccagctgtttgggaggctgaggcaggagaatcacttgaacctgggaggcggaggctgcagtgaagtgagaatgcaccactgcactccagcctaggtgacagagtgagactccgtctcaaataaaaaaatatatatagccaaacagagaaactaaagtacactaaaatacggtaatataatcttatgggaccactgtcataaatgcagtccagtgctaaccaaaatgtcattatgcggtgcatgattgtatattaaaaagattactgttgtaagagtcagaatgcagtcatcccttcagtaaataattattcggggtagtctactagatacttgggatacaactcatcctaggagctcatagtgtagttagaagatgtgggtttcaataccagtatagcaaccagcccaatgactttgactaatacattttaaatttctgcgagcctcctgttttctctactacaaaataaaaacatatattgcctagactgaagtattatgaaaattatgaggtaatatgtgaaaacacattatgaattataaagccttatacaaatgtaaaagaatcaagtatctctaaaatatatgtgagaaatatttaggaaataaagcattggaaaacactaagttagaaataattacaaaagcactcagttaagcaaattaaaatagttataatcaggatcataaataaaaaatataagttttgcattattctatttccttggactaagagttctcactgacattagaagataaaataattaactgaaagatgtaaaatcttgttcgtaatctgtgatttgtaggctactaaaataaatggatggattattgccacacttaccctacaggtaaaaaatgatgtagaataagatcaagctttcttggttcttgacagaaatgtctgaagagcaaaggtgtccacagaatgacagctgtgggacgaattatgaaggcaagtgccaccagggatgagtatttgacactataagaggaaaatgtgtgataagacaagtgggaaattaacttgaagttatctatcatttacactgtgatttgacattttccttttggtataaatagctatatgaacttttttttgttttgttttgttttttttgagacagagtctcactctgtcacccaggctggagtgcagtggcatgatcttggctcacggcaacttctgcctcccaggtgcaagtgattctcctgcctcagcatcccgagtagctgggattacaggtgtgtaccaccatgcccgactaattttttgtattttcagtagagatggggtttcaccttgttggccaggctggcctcaaactcctgacgtcaggtgatctgcccacctcagcctcctaaagtgctgggattacaggctaatccccatgccaggctaattttttgtatttttagtgggagacagggttttaccatgttggccaggctggtctcaaactcctgacctcaggcgatccgccagcctcagcctcctaaagtgctgggattacaggcaggagccacagtgcctggcctaaatagctatattaacttttgcaggttacttacgctttctgggtcttagtttttcttatcttttaaaaaaggtaatatctaaagccgttttcagtgctcatcttctaattctagtattttcaaataagaagttatcatatgtcttaatgccagtcttgtgtgctaaaaagcatgctttttacctcataactttgccaattactttcttgattgaaaaaaaaatgatcaagtttctcctaaggagctataagaaagtcataaagagaggctttctctaagaaaaagcatgagcatttaaagaaacaggattatttaggcagtgatgggctcagagtatttagaattttgttttgaaagttaattttagccaagtgaaatatatatgcatacaactgataaaaggcctactgtataaactgtatgatcatggatagaaatacagatttttattaacaggacttctctaaactaatggttgaaagagacaatttgataatttaatcatgaggactatggatagtatattattatgtggaaaaatacttagtcctgtaaaaaattaaaagaaagcaaaataagacaacctatattaccattatatacctattaactaataaaaataactataaagaaatctgtaggccaggtgtggtggctcaggcctgcaatcccagcactttgggaggccgaggtgggtggatcacctgaagtcaggagttcaagaccagtctggccaaaatggtgaaaccctgtctctactaaagatagaaaaaaattagctgggtgtggtggcaggtgcctgtaatcccagctacttgggaggctgaggcaggagaatcacttgaacctgggaagtggaggttgcaatgagctgagatcatgccattgcactctagcctgggcaacaagaatgaaactccatctcaaaaaaaagaaaaaaaaaaaagaatgaattc

SEQ ID NO: 11 - DNA, 25 bp, Artificial Sequence
  Description of Artificial Sequence RT-PCR primer
gttgacagaatgctgttccacgtcg

SEQ ID NO: 12 - DNA, 25 bp, Artificial Sequence
  Description of Artificial Sequence RT-PCR primer
caagctgaggcacgaagagcaatga

SEQ ID NO: 13 - protein, 4 aa, Homo sapiens
  C-terminal tetrapeptide
ELKS